(19) United States
(12) Patent Application Publication
(10) Pub. No.: US 2025/0051361 A1
(43) Pub. Date: Feb. 13, 2025

(54) 5,6,7,8-TETRAHYDRO-2,6-NAPHTHYRIDINE DERIVATIVES AS CANCER THERAPEUTICS

(71) Applicant: VRise Therapeutics, Inc., Cambridge, MA (US)

(72) Inventors: Prashant Kashinath BHAVAR, Hyderabad (IN); Anuj Ramesh KSHIRSAGAR, Hyderabad (IN); Partha Pratim SARMA, Hyderabad (IN); Uday Kumar SURAMPUDI, Cambridge, MA (US)

(21) Appl. No.: 18/716,825

(22) PCT Filed: Dec. 9, 2022
(86) PCT No.: PCT/IB2022/062015

§ 371 Date: Jun. 5, 2024

(30) Foreign Application Priority Data
Dec. 10, 2021 (IN) 202141057591

(51) Int. Cl. C07D 519/00; A61K 31/496; A61K 31/4995; A61K 31/55; A61P 35/00; C07D 471/04

(52) U.S. Cl. CPC C07D 519/00; A61K 31/496; A61K 31/4995; A61K 31/55; A61P 35/00; C07D 471/04

(57) ABSTRACT

The present invention relates to novel compounds described herein, the method of preparing the same, its pharmaceutical composition and method for use thereof. In particular the invention to compounds of formula A or their pharmaceutically acceptable salts thereof are inhibitors of KRAS protein and useful in treatment, prevention and/or amelioration of diseases or disorders associated with KRAS especially the Cancer.

This application claims the benefit of priority to Indian provisional patent application number 202141057591 filed dated 10th December 2021 which is hereby incorporated by reference in its entirety.

### FIELD OF THE INVENTION

The present invention relates to novel compounds described herein, the method of preparing the same, its pharmaceutical composition and method for use thereof. In particular the invention to compounds of formula A or their pharmaceutically acceptable salts thereof are inhibitors of KRAS protein and useful in treatment, prevention and/or amelioration of diseases or disorders associated with KRAS especially the Cancer.

### BACKGROUND OF THE INVENTION

Genes are in the DNA of each cell of human body that control how the cell functions, including: how quickly it grows, how often it divides or how long it lives. Genes control how your cells work by making proteins. The proteins have specific functions and act as messengers for the cell and each gene must have the correct instructions for making its protein. This allows the protein to perform the correct function for the cell. All cancers begin when one or more genes in a cell mutate. A mutation is a change. It creates an abnormal protein. Or it may prevent a protein's formation. An abnormal protein provides different information than a normal protein. This can cause cells to multiply uncontrollably and become cancerous.

Broadly the two basic types of genetic mutations that are referred to by researchers are (a) Acquired mutations and factors that cause these mutations include use of Tobacco, exposure to Ultraviolet (UV) radiation, Viruses or age. Cancer that occurs because of acquired mutations is called sporadic cancer; and (b) Germline mutations: A germline mutations are less common and occurs in a sperm cell or egg cell. Because the mutation affects reproductive cells, it can pass from generation to generation. Cancer caused by germline mutations is called inherited cancer and accounts for about 5% to 20% of all cancers.

Many of the genes that contribute to cancer development fall into broad categories:

DNA repair genes. These fix mistakes made when DNA is copied. Many of them function as tumor suppressor genes. BRCA1, BRCA2, and p53 are all DNA repair genes. If a person has an error in a DNA repair gene, mistakes remain uncorrected and the mistakes may lead to mutations. These mutations may eventually lead to cancer, particularly is the said mutations occurs in tumor suppressor genes or oncogenes.

Tumor suppressor genes. These are protective genes. Normally, they limit cell growth by monitoring how quickly cells divide into new cells, repairing mismatched DNA and controlling cell death. When a tumor suppressor gene mutates, cells grow uncontrollably, and they may eventually form a tumor. Examples of tumor suppressor genes include BRCA1, BRCA2, and p53 or TP53.

Oncogenes. These turn a healthy cell into a cancerous cell. Mutations in these genes are not known to be inherited. The two most common oncogenes are HER2, a specialized protein that controls cancer growth and spread. It is found in some cancer cells. For example, breast and ovarian cancer cells and RAS, the gene of RAS family, which makes proteins involved in cell communication pathways, cell growth, and cell death.

One such mutation that has been reported, is the mutation in RAS family of genes. RAS is been known to acts as a molecular switch and is a monomeric globular proteins that is associated with the plasma membrane. RAS can either bind to guanosine 5′-diphosphate (GDP) (known as a Resting or in inactive state) or guanosine-5′-triphosphate (GTP) and converts GDP to GTP (known as a “switched on” or in active state). It is the growth promoting stimuli that causes the induction of RAS wherein the exchange of GDP to GTP occurs there by allowing the active state of RAS to interact with and activate other proteins. This activation of RAS provides a signal to the cell to continue to grow and divide.

It is noteworthy to mention, the Intrinsic ability of RAS protein to turn off by switching back to GDP from GTP is very low and for RAS to turn off it requires GTPase-activating proteins (GAPs) which interact with RAS and greatly accelerate the conversion of GTP to GDP. Any mutation in RAS which affects its ability to interact with GAP or to convert GTP back to GDP result in a prolonged activation of the protein and consequently a prolonged and overactive RAS signaling ultimately lead to cancer.

RAS family is further divided in several members such as HRAS; KRAS; DIRAS1; DIRAS2; DIRAS3; ERAS; GEM; MRAS; NKIRAS1; NKIRAS2; NRAS; RALA; RALB; RAPlA; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS however the most notable RAS members associated with cancers are Harvey rat sarcoma viral oncogene homolog (HRAS) Kirsten rat sarcoma viral oncogene homolog (KRAS) and Neuroblastoma rat sarcoma viral oncogene homolog (NRAS).

With the three HRAS, KRAS and NRAS members, majority of mutations of around 25-30% in tumors are detected in KRAS gene with around 30% of all human tumors been reported to have some mutation to RAS gene.

Mutation of KRAS gene are more common in pancreatic cancer, lung adenocarcinoma, colorectal cancer, gall bladder cancer, thyroid cancer, and bile duct cancer. KRAS mutations have also been seen in about 25% of patients with NSCLC, and some studies have indicated that KRAS mutations are a negative prognostic factor in patients with NSCLC. Recently, KRAS mutations have been found to confer resistance to epidermal growth factor receptor (EGFR) targeted therapies in colorectal cancer; Understanding the status of KRAS mutation seems to be gaining importance prior to use of tyrosine kinase inhibitors (TKI).

The most common KRAS mutations are found in the P-loop at residue G12 and G13 and at residue Q61 with G12D been a frequent mutation of KRAS gene. G12D mutation have been reported to be most prevalent across several cancers with majority been reported in Pancreatic, colon and lung cancer.

Researchers have learned a lot about how cancer genes work. But many cancers are not linked with a specific gene. Cancer likely involves multiple gene mutations. Moreover, some evidence suggests that genes interact with their environment. This further complicates our understanding of the role genes play in cancer. Researchers continue to study how genetic changes affect cancer development. This knowledge has led to improvements in cancer care, including early detection, risk reduction, the use of targeted therapy, and survival.

We believe, Cancer in general have very few options of treatment, especially when the cancer is a result of metastasis and is unresectable. On one end there have been several advancement in options of treatment for example use of chemotherapy either alone or in combination with radiation and/or surgery however on the other side there has been a significant amount of challenges with prognosis especially for the cancers such as the lung cancers, pancreatic cancer, prostate cancer, gastric cancer, endometrial cancer, ovarian cancer, colorectal cancer.

Accordingly, there exists an unmet medical need for treatments for such cancers and the present invention aims to address the same.

### SUMMARY OF THE INVENTION

The present invention relates to compounds of formula (A), or pharmaceutically acceptable salts or compositions and methods of treatment with them. In particular the present invention relates to compounds of formula (A) and their pharmaceutically acceptable salts thereof useful in the treatment of RAS mediated cancer.

In one embodiment of the invention, the substituted naphthyridine compounds represented by structural formula (A)

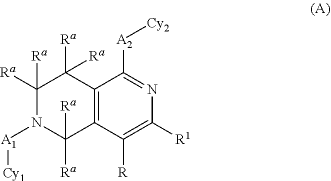

or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—. O, S, —S(═O)P—, —C(═O)—NR^{x}—, —CO—NR^{x}— and —NRₓ—CO—;
A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, O, S, —S(═O)P—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G;
G is a group capable of forming an interaction with Aspartic acid at 12 position of KRAS protein;
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NbC(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{a} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S and
each occurrence of p is independently 0, 1 or 2.

Further preferred are compounds having the formula (A), wherein A₂ is absent or is —CR^{b}R^{c}—; wherein each of R^{b} and R^{c} is independently selected from hydrogen, substituted or unsubstituted alkyl

Further preferred are compounds having the formula (A), wherein A₂ is absent or is —CR^{b}R^{c}—; where in R^{b} is independently methyl or ethyl and R^{c} is hydrogen.

Further preferred are compounds having the formula (A), wherein A₂ is absent.

Further preferred are compounds having the formula (A), wherein R is Cyano (CN).

In one embodiment of the invention, the substituted naphthyridine compounds represented by structural formula (A-I)

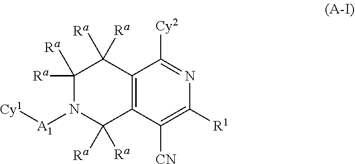

—or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G;
G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{b} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2.

Further preferred are compounds having the formula (A) or (A-I), wherein Cy² is selected from cyclic group selected from substituted or unsubstituted cycloalkyl, substituted or unsubstituted cycloalkylalkyl, substituted or unsubstituted heterocyclyl and substituted or unsubstituted heterocyclylalkyl.

Further preferred are compounds having the formula (A) or (A-I), wherein Cy² is selected from cyclic group selected from substituted or unsubstituted cycloalkyl, substituted or unsubstituted cycloalkylalkyl, substituted or unsubstituted heterocyclyl and substituted or unsubstituted heterocyclylalkyl, wherein each group is optionally further substituted with group G.

In one embodiment of the invention, the substituted naphthyridine compounds represented by structural formula (A-II)

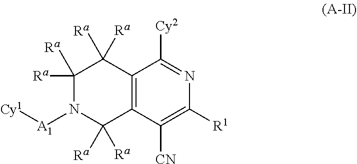

or a tautomer thereof, isotope thereof, prodrug thereof, N-oxide thereof, a pharmaceutically acceptable ester thereof, a pharmaceutically acceptable salt thereof, or a stereoisomer thereof, wherein
   A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
   Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
   Cy² is substituted or unsubstituted heterocyclyl, which is optionally substituted by G;
   G is a group capable of forming an interaction with Aspartic acid at 12 position of KRAS protein;
   R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NbC(═O)NRR^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
      R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRWS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
   each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
   each occurrence of p is independently 0, 1 or 2.

Further preferred are compounds having the formula (A), (A-I) or (A-II), wherein Cy² is selected from

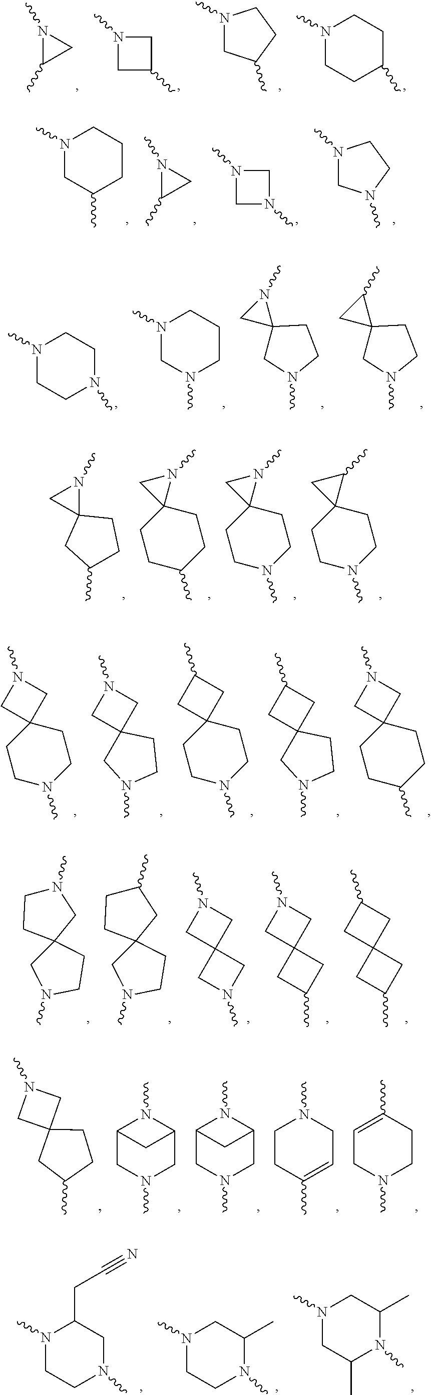

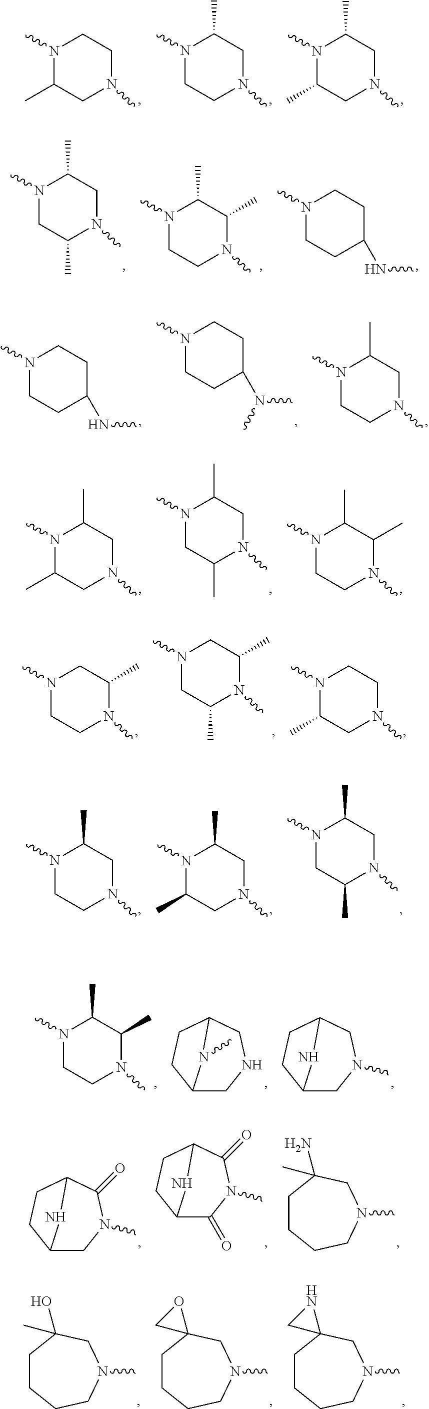

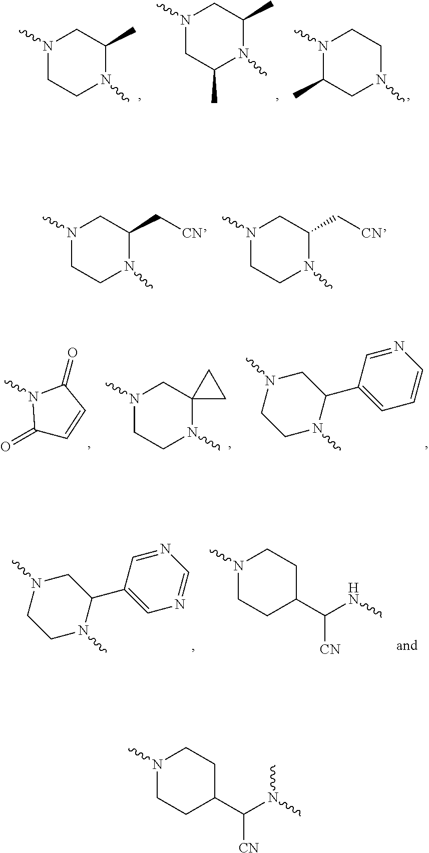

wherein the Cy² is substituted with one or more G.

Further preferred are compounds having the formula (A), (A-I) or (A-II), wherein A₂—Cy²-G is selected from

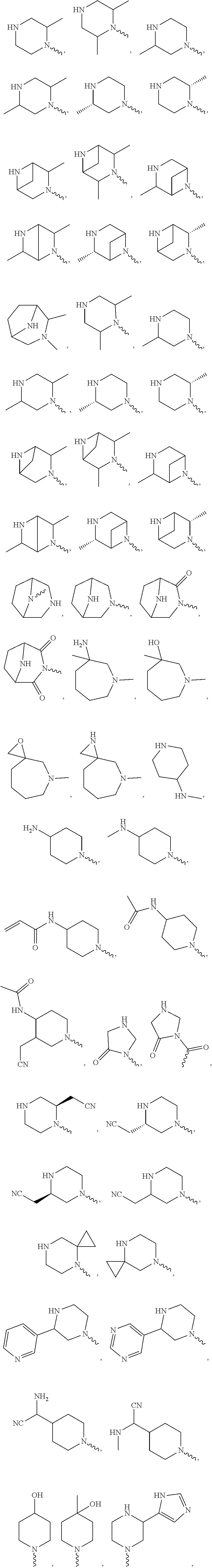

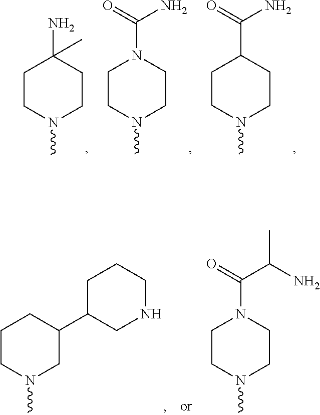

Further preferred are compounds having the formula (A), (A-I) or (A-II), wherein A₁ is
(i) absent or is —CR^{b}R^{c}—; wherein each of R^{b} and R^{c} are independently selected from hydrogen or substituted or unsubstituted alkyl and/or
(ii) absent or is —CR^{b}R^{c}—; wherein R^{b} is independently methyl or ethyl and R^{c} is hydrogen and/or
(iii) absent or is —CR^{b}R^{c}—; wherein R^{b} is hydrogen and R^{c} is hydrogen and/or (iv) absent or —CH₂ or —(C═O) and/or any combinations thereof.

A₂ is
(i) absent or is —CR^{b}R^{c}—; wherein each of R^{b} and R^{c} are independently selected from hydrogen, substituted or unsubstituted alkyl; and/or
(ii) absent or is —CR^{b}R^{c}—; where in R^{b} is independently methyl or ethyl and R^{c} is hydrogen and/or
(iii) absent and/or any combinations thereof;

Cy² is selected form
(i) a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl and/or
(ii) a cyclic group selected from substituted or unsubstituted aryl and substituted or unsubstituted heteroaryl and/or combinations thereof

Cy² is selected from
(i) cyclic group selected from substituted or unsubstituted cycloalkyl, substituted or unsubstituted cycloalkylalkyl, substituted or unsubstituted heterocyclyl and substituted or unsubstituted heterocyclylalkyl and/or
(ii) cyclic group selected from substituted or unsubstituted cycloalkyl, substituted or unsubstituted cycloalkylalkyl, substituted or unsubstituted heterocyclyl and substituted or unsubstituted heterocyclylalkyl, wherein each group is optionally further substituted with group G and/or combinations thereof, wherein G is a group capable of forming an interaction with Aspartic acid at 12 position of KRAS protein

R is Cyano (CN); and

R¹ is selected from
(i)hydrogen, halogen, OR^{b}, S—R^{b}, —S(═O)pR^{b}—, —C(═O)—R^{b}—NR^{b}R^{c}, —CO—NR^{b}R^{c}—and —NR^{b}—CO—R^{c}; and/or
(ii) —O—R^{b} and/or
(iii) —NR^{b}R^{c} and/or
(iv) substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl and/or
(v) substituted or unsubstituted alkyl, substituted or unsubstituted heterocyclyl, substituted or unsubstituted heterocyclylalkyl and substituted or unsubstituted heteroaryl and/or combinations thereof.

Further preferred are compounds having the formula (A), (A-I) or (A-II), wherein R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NbC(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.

A₁ is absent or substituted or unsubstituted C₁₋₄ alkyl; A₂ is absent or substituted or unsubstituted C₁₋₄ alkyl Cy¹ is selected form substituted or unsubstituted aryl or substituted or unsubstituted heteroaryl; Cy² is selected from substituted or unsubstituted heterocyclyl, which is optionally substituted by G.

G is a group capable of forming an interaction with Aspartic acid at 12 position of KRAS protein.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{c}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}. R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRWS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S.
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I) or (A-II), wherein
A₁ is absent or substituted or unsubstituted alkyl;
A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G.

G is a group capable of forming an interaction with Aspartic acid at 12 position of KRAS protein;
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONRR^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NbC(═O)NRR^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRWS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and each occurrence of p is independently 0, 1 or 2.

Further preferred are compounds having the formula (A), (A-I) or (A-II), wherein
Cy¹ is selected from substituted or unsubstituted aryl or substituted or unsubstituted heteroaryl;
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G; G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}; R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I) or (A-II), wherein
Cy² is substituted or unsubstituted heterocyclyl, which is optionally substituted by G;
G is a group capable of forming an interaction with Aspartic acid at 12 position of KRAS protein;
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NbC(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NbC(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I) or (A-II), wherein
R¹ is selected from hydrogen, halogen, —NR^{b}R^{c} or —OR^{b};
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—; A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G;
G is a group capable of forming an interaction with Aspartic acid at 12 position of KRAS protein;
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NbC(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

In another embodiment of the invention, the substituted naphthyridine compounds represented by structural formula (A-IIIA), (A-IIIB) or (A-IIIC)

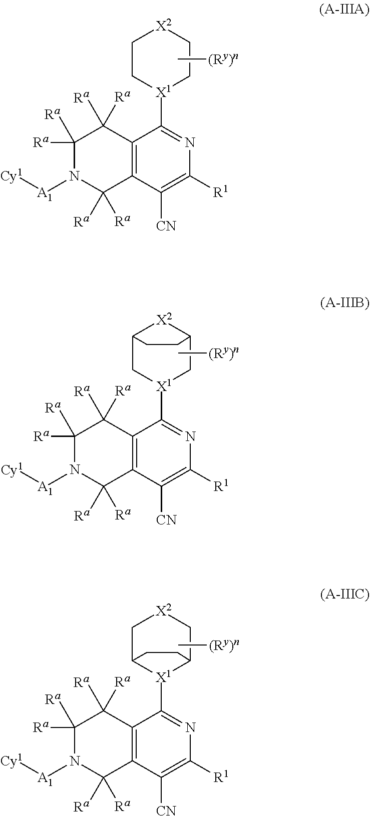

or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
X¹ is C or N;
X² is selected from —N-G or CR^{y}R^{y}-G, O or S;
G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NRR^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NbC(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{y} is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NbC(═O)NR^{b}R^{c}, —NR^{b}S(═O)R^{c}, —NR^{b}S(═O)₂R^{c}, —NR^{b}—OR^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
n is 0, 1, 2, 3, 4, 5, 6, 7 or 8; and
   each occurrence of p is independently 0, 1 or 2.

Further preferred are compounds having the formula (A), (A-I), (A-II), (A-IIIA), (A-IIIB) or (A-IIIC), wherein A₁ is absent or is —CR^{b}R^{c}—; wherein each of R^{b} and R^{c} is independently selected from hydrogen or substituted or unsubstituted alkyl.

Further preferred are compounds having the formula (A), (A-I), (A-II), (A-IIIA), (A-IIIB) or (A-IIIC), wherein A₁ is absent or is —CR^{b}R^{c}—; wherein R^{b} is independently methyl or ethyl and R^{c} is hydrogen.

Further preferred are compounds having the formula (A), (A-I), (A-II), (A-IIIA), (A-IIIB) or (A-IIIC), wherein A₁ is absent or is —CR^{b}R^{c}—; wherein R^{b} is hydrogen and R^{c} is hydrogen.

Further preferred are compounds having the formula (A), (A-I), (A-II), (A-IIIA), (A-IIIB) or (A-IIIC), wherein A₁ is absent or —CH₂ or —(C═O).

Further preferred are compounds having the formula (A), (A-I), (A-II), (A-IIIA), (A-IIIB) or (A-IIIC), wherein Cy¹ is selected form a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl.

Further preferred are compounds having the formula (A), (A-I), (A-II), (A-IIIA), (A-IIIB) or (A-IIIC), wherein Cy¹ is selected form a cyclic group selected from substituted or unsubstituted aryl and substituted or unsubstituted heteroaryl.

Further preferred are compounds having the formula (A), (A-I), (A-II), (A-IIIA), (A-IIIB) or (A-IIIC), wherein Cy¹ is selected form

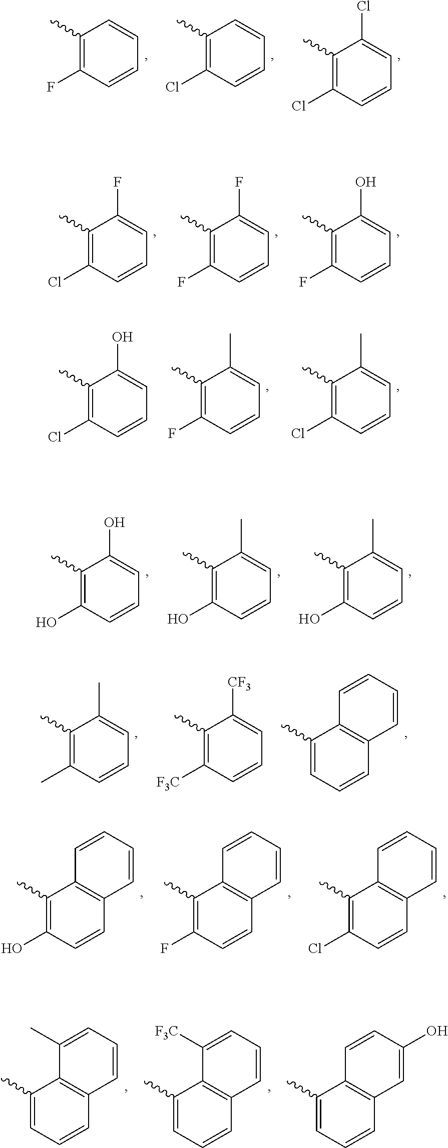

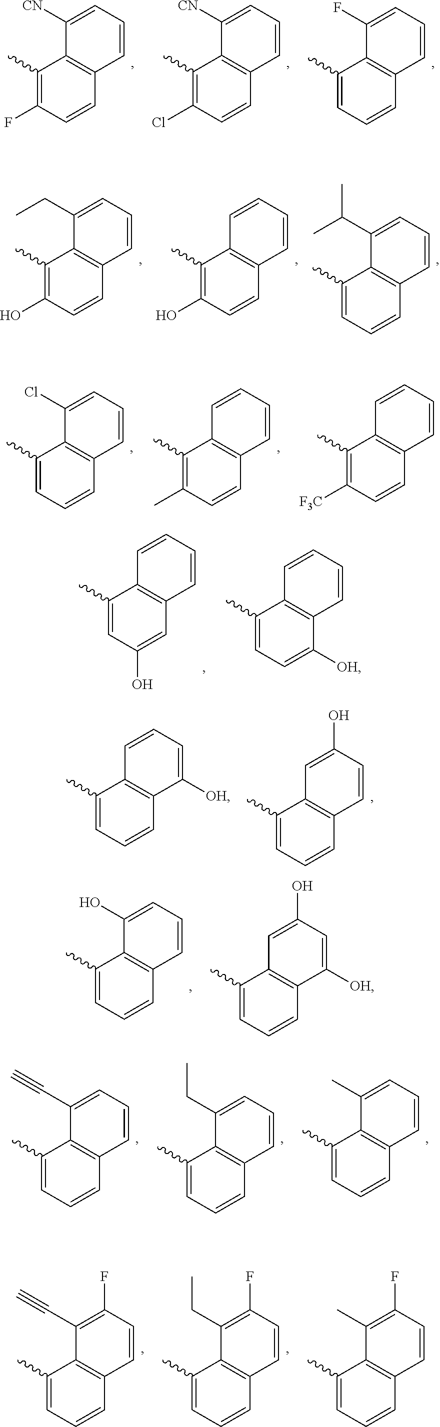

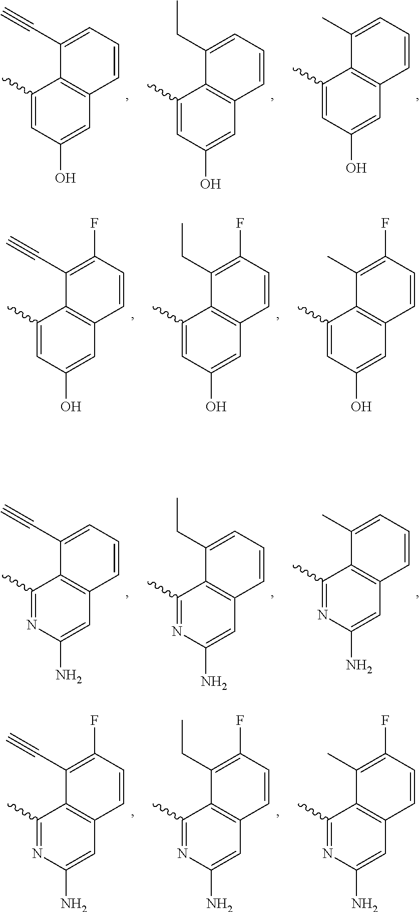

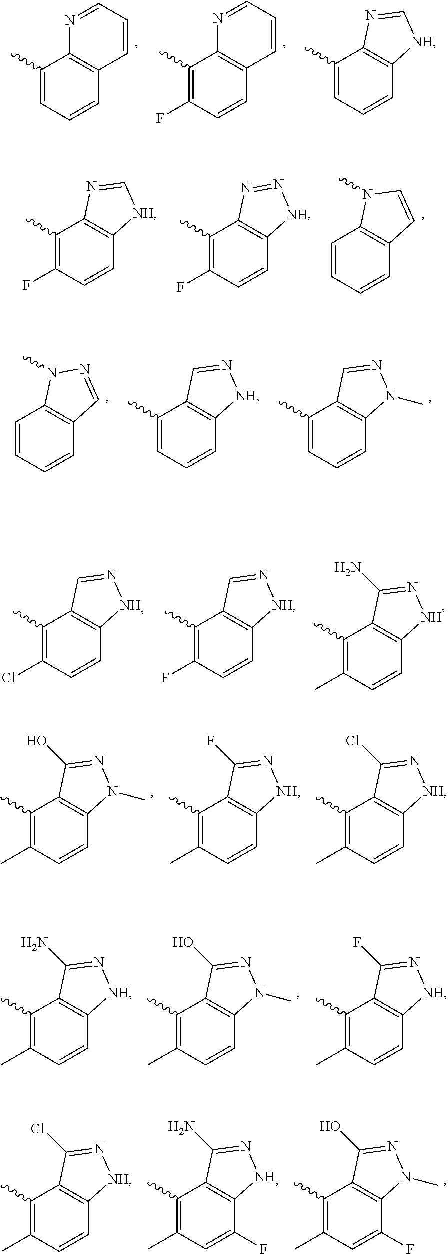

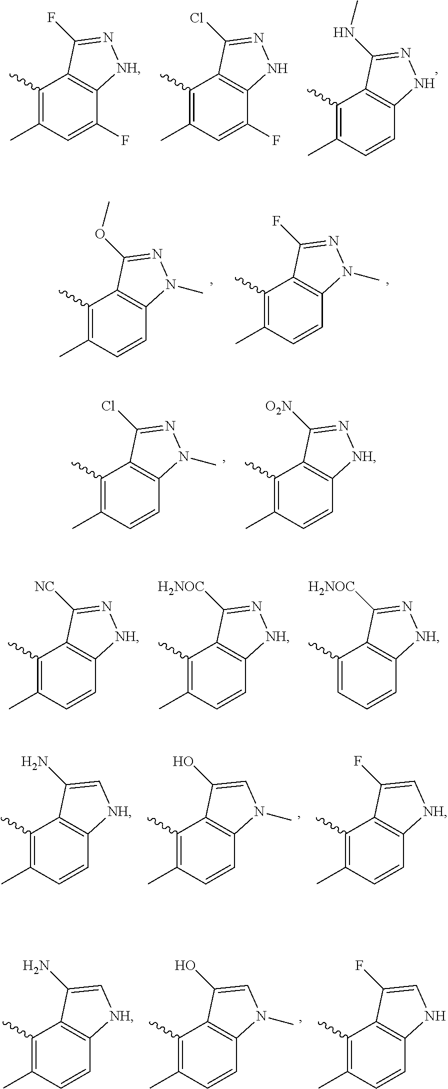

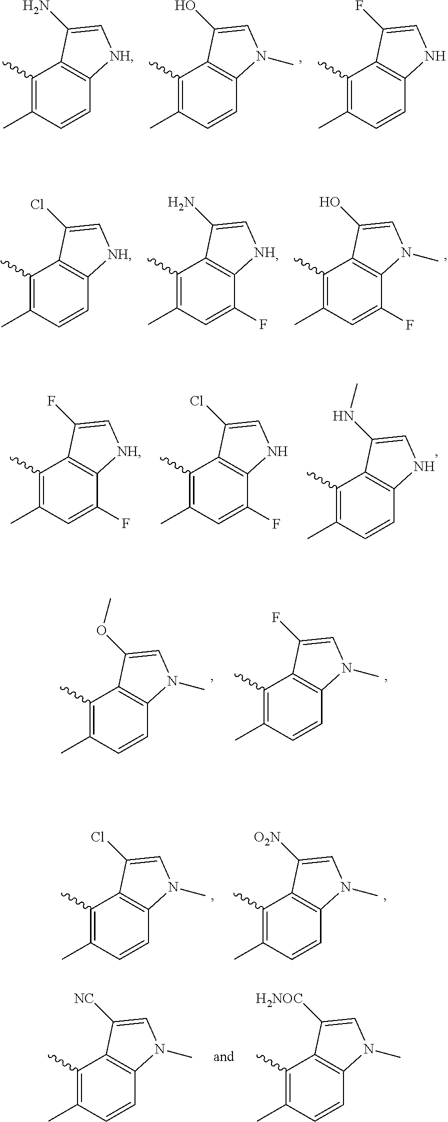

Further preferred are compounds having the formula (A), (A-I), (A-II), (A-IIIA), (A-IIIB) or (A-IIIC), wherein G is Hydrogen, —NH₂, —NHR^{a}, —NHOH, —COOH, —OH or —CHR^{a}—OH, or —CH₂—OH or amino acid,

Further preferred are compounds having the formula (A), (A-I), (A-II), (A-IIIA), (A-IIIB) or (A-IIIC), wherein G is Hydrogen, D-valine or L-threonine.

Further preferred are compounds having the formula (A), (A-I), (A-II), (A-IIIA), (A-IIIB) or (A-IIIC), wherein R¹ is selected from hydrogen, halogen, OR^{b}, S—R^{b}, —S(═O)pR^{b}—, —C(═O)—R^{b}, —NR^{b}R^{c}, —CO—NR^{b}R^{c}— and —NR^{b}—CO—R^{c}.

Further preferred are compounds having the formula (A), (A-I), (A-II), (A-IIIA), (A-IIIB) or (A-IIIC), wherein R¹ is —O—R^{b}.

Further preferred are compounds having the formula (A), (A-I), (A-II), (A-IIIA), (A-IIIB) or (A-IIIC), wherein R¹ is —NR^{b}R^{c}.

Further preferred are compounds having the formula (A), (A-I), (A-II), (A-IIIA), (A-IIIB) or (A-IIIC), wherein R¹ is independently selected from, substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl.

Further preferred are compounds having the formula (A), (A-I), (A-II), (A-IIIA), (A-IIIB) or (A-IIIC), wherein R¹ is independently selected from substituted or unsubstituted alkyl, substituted or unsubstituted heterocyclyl, substituted or unsubstituted heterocyclylalkyl and substituted or unsubstituted heteroaryl.

Further preferred are compounds having the formula (A), (A-I), (A-II), (A-IIIA), (A-IIIB) or (A-IIIC), wherein R¹ is independently selected from

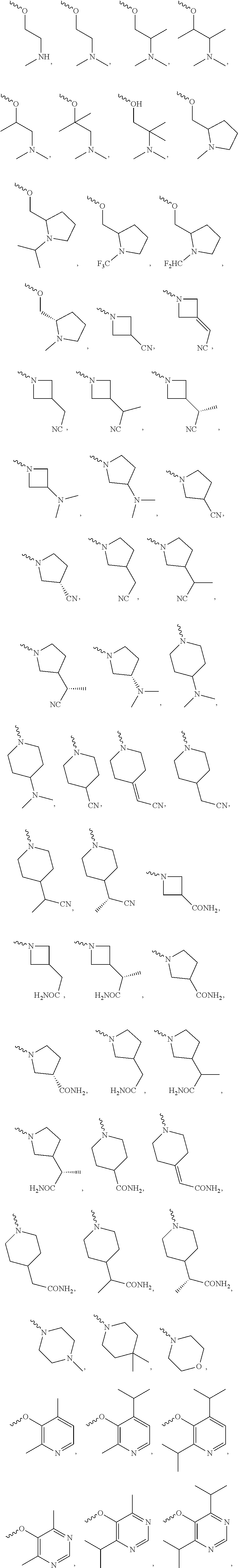

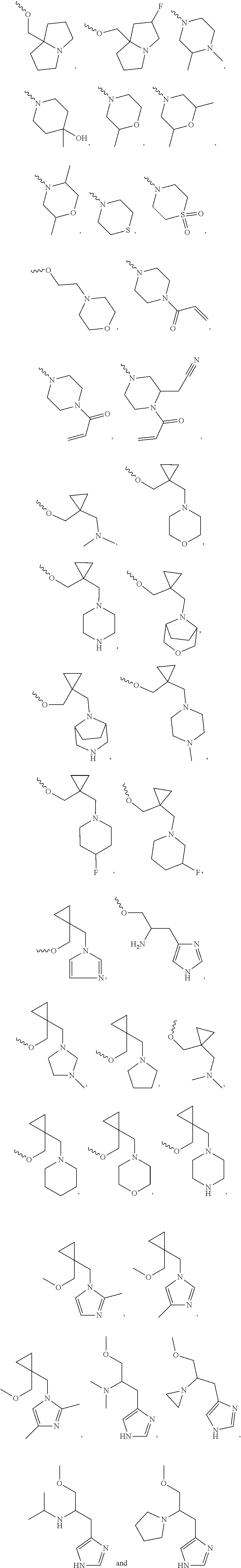

Further preferred are compounds having the formula (A), (A-I), (A-II), (A-IIIA), (A-IIIB) or (A-IIIC), wherein R^{a} is Hydrogen or substituted or unsubstituted alkyl.

Further preferred are compounds having the formula (A), (A-I), (A-II), (A-IIIA), (A-IIIB) or (A-IIIC), wherein two R^{a} attached to same carbon atom form a C═O (Oxo) group.

Further preferred are compounds having the formula (A), (A-I), (A-II), (A-IIIA), (A-IIIB) or (A-IIIC), wherein A₁ is
(i) absent or is —CR^{b}R^{c}—; wherein each of R^{b} and R^{c} are independently selected from hydrogen or substituted or unsubstituted alkyl and/or
(ii) absent or is —CR^{b}R^{c}—; wherein R^{b} is independently methyl or ethyl and R^{c} is hydrogen and/or
(iii) absent or is —CR^{b}R^{c}—; wherein R^{b} is hydrogen and R^{c} is hydrogen and/or (iv) absent or —CH₂ or —(C═O) and/or any combinations thereof.

Cy¹ is selected from
(i) a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl and/or
(ii) a cyclic group selected from substituted or unsubstituted aryl and substituted or unsubstituted heteroaryl and/or combinations thereof;
   R¹ is selected from
(vi) hydrogen, halogen, OR^{b}, S—R^{b}, —S(═O)pR^{b}—, —C(═O)—R^{b}, —N^{b}R^{c}, —CO—NR^{b}R^{c}— and —NR^{b}—CO—R^{c}; and/or
(vii) —O—R^{b} and/or
(viii) —NR^{b}R^{c} and/or
(ix) substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl and/or
(x) substituted or unsubstituted alkyl, substituted or unsubstituted heterocyclyl, substituted or unsubstituted heterocyclylalkyl and substituted or unsubstituted heteroaryl and/or combinations thereof;
X¹ is C or N;
X² is selected from —N-G or CR^{y}R^{y}-G, O or S;
G is a group capable of forming an interaction with Aspartic acid at 12 position of KRAS protein; and
   each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S.

Further preferred are compounds having the formula (A), (A-I), (A-II), (A-IIIA), (A-IIIB) or (A-IIIC), wherein
A₁ is absent or substituted or unsubstituted C₁₋₄ alkyl;
Cy¹ is selected form substituted or unsubstituted aryl or substituted or unsubstituted heteroaryl;
X¹ is C or N;
X² is selected from —N-G or CR^{y}R^{y}-G, O or S; G is a group capable of forming an interaction with Aspartic acid at 12 position of KRAS protein;
   each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NRR^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I), (A-II), (A-IIIA), (A-IIIB) or (A-IIIC), wherein
A₁ is absent or substituted or unsubstituted alkyl;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
X¹ is C or N;
X² is selected from —N-G or CR^{y}R^{y}-G, O or S;
G is a group capable of forming an interaction with Aspartic acid at 12 position of KRAS protein;
each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I), (A-II), (A-IIIA), (A-IIIB) or (A-IIIC), wherein
Cy¹ is selected from substituted or unsubstituted aryl or substituted or unsubstituted heteroaryl;
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.
   X¹ is C or N;
   X² is selected from —N-G or CR^{y}R^{y}-G, O or S;
G is a group capable of forming an interaction with Aspartic acid at 12 position of KRAS protein;
each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R, —C(═O)NR^{b}R^{c}, —C(═O)ONR^{b}R^{c}, —NR^{b}R^{c}, —NR^{b}C(═O)NRR^{c}, —NR S(═O)R^{c}, —NR S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{c}R^{c}, —R^{b}NR^{c}C(═O)R^{b}, —R^{b}OR^{c}, —R^{b}C(═O)OR^{c}, —R^{c}C(═O)NR^{c}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}C(═O)R^{c}, —SR^{b}, —SOR^{c}, —SO₂R, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I), (A-II), (A-IIIA), (A-IIIB) or (A-IIIC), wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
X¹ is C or N;
X² is selected from —N-G or CR^{y}R^{y}-G, O or S;
G is a group capable of forming an interaction with Aspartic acid at 12 position of KRAS protein;
each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NbC(═O)OR^{c}, —NR^{b}C(═O)R^{c}, —NR^{b}C(═S)R^{c}, —NR^{b}C(═S)NbR^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{c}R^{c}, —R^{b}NR^{c}C(═O)R^{b}, —R^{b}OR^{c}, —R^{b}C(═O)OR^{c}, —R^{c}C(═O)NR^{c}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}C(═O)R^{c}, —SR^{b}, —SOR^{c}, —SO₂R, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S; and
each occurrence of p is independently 0, 1 or 2.

Further preferred are compounds having the formula (A), (A-I), (A-II), (A-IIIA), (A-IIIB) or (A-IIIC), wherein
R¹ is selected from hydrogen, halogen, —NR^{b}R^{c} or —OR^{b};
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
X¹ is C or N;
X² is selected from —N-G or CR^{y}R^{y}-G, O or S;
G is a group capable of forming an interaction with Aspartic acid at 12 position of KRAS protein;
each occurrence of R^{y} is independently selected from hydrogen, hydroxy, halogen, substituted or unsubstituted alkyl, —NR^{b}R^{c}, —OR^{b}, —SR^{b}, —SOR^{c}, —SO₂R^{b}, or two R^{y} may be joined to a form a substituted or unsubstituted saturated or unsaturated 3-6 member ring, which may optionally include heteroatoms which may be same or different and are selected from O, NR^{a} or S;
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} are independently selected from hydrogen, substituted or
unsubstituted alkyl, or variables of R^{b} and R^{c} together with the nitrogen which they attached
can form a substituted or unsubstituted heterocyclic ring;
   each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
   Z′ is selected from O or S; and
   each occurrence of p is independently 0, 1 or 2.

In another embodiment of the invention, the substituted naphthyridine compounds represented by structural formula (A), (A-I), (A-II), (A-IIIA), (A-IIIB) or (A-IIIC),
A₁ is absent or substituted or unsubstituted alkyl;
Cy¹ is selected from substituted or unsubstituted aryl or substituted or unsubstituted heteroaryl;
R¹ is selected from hydrogen, halogen, substituted or unsubstituted alkyl, —NR^{b}R^{c} or —OR^{b}; each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
X¹ is C or N;
X² is selected from —N-G or CR^{y}R^{y}-G, O or S;
   G is a group capable of forming an interaction with Aspartic acid at 12 position of KRAS protein;
R^{y} is selected from hydrogen, halogen, substituted or unsubstituted alkyl; and
n is 0, 1, 2, 3, 4, 5, 6, 7 or 8.

Further preferred are compounds having the formula (A), (A-I), (A-II), (A-IIIA), (A-IIIB) or (A-IIIC), wherein A₁ is absent or methyl.

Further preferred are compounds having the (A), (A-I), (A-II), (A-IIIA), (A-IIIB) or (A-IIIC), wherein A₁ is absent.

Further preferred are compounds having the formula (A), (A-I), (A-II), (A-IIIA), (A-IIIB) or (A-IIIC), wherein Cy¹ is selected from substituted or unsubstituted aryl.

Further preferred are compounds having the formula (A), (A-I), (A-II), (A-IIIA), (A-IIIB) or (A-IIIC), wherein Cy¹ is selected from phenyl or naphthalene, optionally substituted with halogen, hydroxy or substituted or unsubstituted alkyl.

Further preferred are compounds having the formula (A), (A-I), (A-II), (A-IIIA), (A-IIIB) or (A-IIIC), wherein Cy¹ is selected from quinoline or quinazoline, optionally substituted with halogen, hydroxy or substituted or unsubstituted alkyl.

Further preferred are compounds having the formula (A), (A-I), (A-II), (A-IIIA), (A-IIIB) or (A-IIIC), wherein R¹ is selected from hydrogen, halogen, hydroxy,

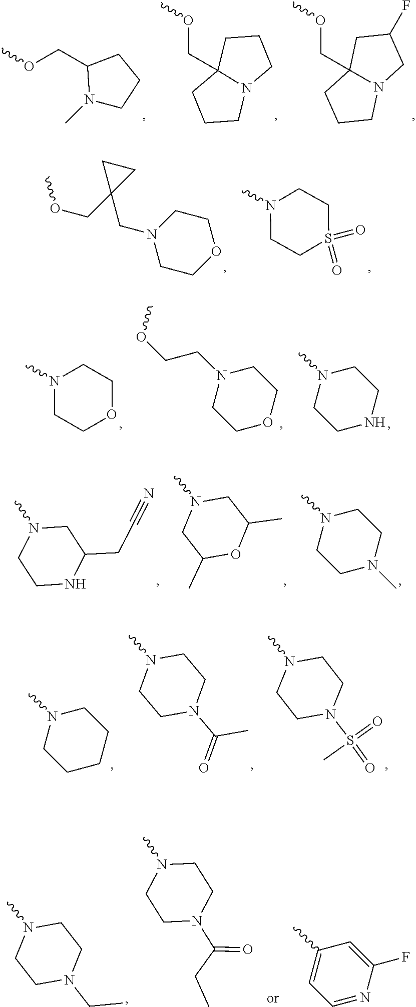

Further preferred are compounds having the formula (A-IIIA), (A-IIIB) or (A-IIIC), wherein X¹ is N.

Further preferred are compounds having the formula (A-IIIA), (A-IIIB) or (A-IIIC), wherein X² is selected from —N-G or CR^{y}R^{y}-G or O, wherein G is selected from Hydrogen, —NH2, —NHR^{a}, —NHOH, —COOH, —OH or —CHR^{a}—OH, or —CH₂—OH or an amino acid.

Further preferred are compounds having the formula (A), (A-I), (A-II), (A-IIIA), (A-IIIB) or (A-IIIC), wherein
A₁ is absent or methyl;
Cy¹ is selected from substituted or unsubstituted aryl;
R¹ is selected from hydrogen, halogen, hydroxy,

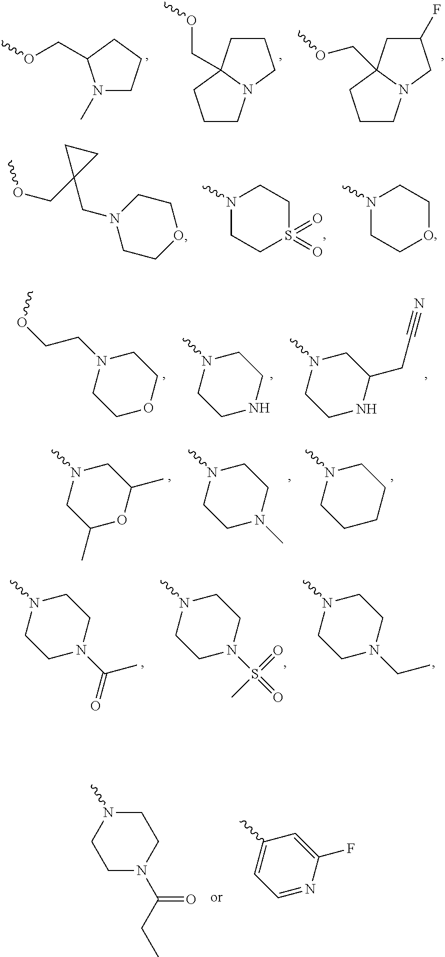

X¹ is N; and
X² is selected from —N-G or CR^{y}R^{y}-G or O, wherein G is selected from Hydrogen, —NH₂, —NHR^{a}, —NHOH, —COOH, —OH or —CHR^{a}—OH, or —CH₂—OH or an amino acid.

Further preferred are compounds having the formula (A), (A-I), (A-II), (A-IIIA), (A-IIIB) or (A-IIIC), wherein A₂—Cy²-G is selected from

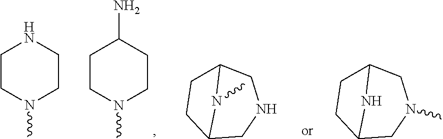

It is to be understood herein that any permutation combination of each of the above disclosed embodiments including permutation combinations of preferred embodiments is envisaged and within the ambit of this invention for e.g. any permutation combinations of different variables described herein can be chosen to be part of the invention.

The non-limiting representative compounds of the present invention are as listed herein below and pharmaceutically acceptable salts thereof. The present invention should not be construed to be limited to these compounds.
1. 1-(3,8-diazabicyclo[3.2.1]octan-3-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile hydrochloride;
2. tert-butyl 4-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate;
3. 6-(8-chloronaphthalen-1-yl)-1-(3-(cyanomethyl)piperazin-1-yl)-3-(((S)-1-methylpyrrolidin-2-yl)methoxy)-5,6,7,8-tetrahydro-2,6-naphthyridine-4-carbonitrile hydrochloride;
4. tert-butyl (1R,5S)-8-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3, 8-diazabicyclo[3.2.1]octane-3-carboxylate;
5. 1-((1R,5S)-3, 8-diazabicyclo[3.2.1]octan-8-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile hydrochloride;
6. 1-((1R,5S)-3, 8-diazabicyclo[3.2.1]octan-8-yl)-6-(3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
7. 1-((1R,5S)-3, 8-diazabicyclo[3.2.1]octan-8-yl)-6-(8-bromonaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
8. 1-((1R,5S)-3, 8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
9. 1-((1R,5S,8r)-3-azabicyclo[3.2.1]octan-8-yl)-6-(8-bromo-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile;
10. 1-((1R,5S)-3, 8-diazabicyclo[3.2.1]octan-8-yl)-6-(8-bromonaphthalen-1-yl)-3-((1-methylpyrrolidin-2-yl)methoxy)-5,6,7,8-tetrahydro-2,6-naphthyridine-4-carbonitrile;
11. 1-((1R,5S)-3, 8-diazabicyclo[3.2.1]octan-8-yl)-6-(8-ethynylnaphthalen-1-yl)-3-(((S)-1-methylpyrrolidin-2-yl)methoxy)-5,6,7,8-tetrahydro-2,6-naphthyridine-4-carbonitrile;
12. 1-((1R,5S)-3, 8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile;
13. 6-benzyl-1-((1R,5S)-3,8-diazabicyclo[3.2.1]octan-8-yl)-3-((1-(morpholinomethyl)cyclopropyl)methoxy)-5,6,7,8-tetrahydro-2,6-naphthyridine-4-carbonitrile;
14. 1-(3,8-diazabicyclo[3.2.1]octan-3-yl)-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile;
15. 1-(3,8-diazabicyclo[3.2.1]octan-3-yl)-6-(8-bromo-3-hydroxynaphthalen-1-yl)-3-(((S)-1-methylpyrrolidin-2-yl)methoxy)-5,6,7,8-tetrahydro-2,6-naphthyridine-4-carbonitrile;
16. 1-(3,8-diazabicyclo[3.2.1]octan-3-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
17. 1-(3,8-diazabicyclo[3.2.1]octan-3-yl)-6-(8-chloronaphthalen-1-yl)-3-(((2R)-2-fluorotetrahydro-1H-pyrrolizin-7a(5H)-yl)methoxy)-5,6,7,8-tetrahydro-2,6-naphthyridine-4-carbonitrile;
18. 6-benzyl-1-(3,8-diazabicyclo[3.2.1]octan-3-yl)-3-(((S)-1-methylpyrrolidin-2-yl)methoxy)-5,6,7,8-tetrahydro-2,6-naphthyridine-4-carbonitrile;
19. 6-benzyl-1-(3,8-diazabicyclo[3.2.1]octan-3-yl)-3-(((2R)-2-fluorotetrahydro-1H-pyrrolizin-7a(5H)-yl)methoxy)-5,6,7,8-tetrahydro-2,6-naphthyridine-4-carbonitrile;
20. tert-butyl ((R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-methyl-1-oxobutan-2-yl)carbamate;
21. 1-(4-(D-valyl)piperazin-1-yl)-6-benzyl-3-(((S)-1-methylpyrrolidin-2-yl)methoxy)-5,6,7,8-tetrahydro-2,6-naphthyridine-4-carbonitrile;
22. tert-butyl ((2S,3R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-(benzyloxy)-1-oxobutan-2-yl)carbamate;
23. 6-benzyl-1-(4-(O-benzyl-L-threonyl)piperazin-1-yl)-3-(((S)-1-methylpyrrolidin-2-yl)methoxy)-5,6,7,8-tetrahydro-2,6-naphthyridine-4-carbonitrile;
24. 6-benzyl-1-(3,8-diazabicyclo[3.2.1]octan-3-yl)-3-((1-(morpholinomethyl)cyclopropyl)methoxy)-5,6,7,8-tetrahydro-2,6-naphthyridine-4-carbonitrile;
25. 1-(3,8-diazabicyclo[3.2.1]octan-3-yl)-3-(((2R)-2-fluorotetrahydro-1H-pyrrolizin-7a(5H)-yl)methoxy)-6-(3-hydroxynaphthalen-1-yl)-5,6,7,8-tetrahydro-2,6-naphthyridine-4-carbonitrile;
26. 3-(1,1-dioxidothiomorpholino)-6-(naphthalen-1-yl)-1-(piperazin-1-yl)-5,6,7,8-tetrahydro-2,6-naphthyridine-4-carbonitrile Hydrochloride;
27. 6-(8-chloronaphthalen-1-yl)-3-morpholino-1-(piperazin-1-yl)-5,6,7,8-tetrahydro-2,6-naphthyridine-4-carbonitrile Hydrochloride;
28. 3-(2-morpholinoethoxy)-6-(naphthalen-1-yl)-1-(piperazin-1-yl)-5, 6,7,8-tetrahydro-2,6-naphthyridine-4-carbonitrile Hydrochloride;
29. 6-(8-chloronaphthalen-1-yl)-3-(2-morpholinoethoxy)-1-(piperazin-1-yl)-5,6,7,8-tetrahydro-2,6-naphthyridine-4-carbonitrile Hydrochloride;
30. 1-(2,6-dimethylmorpholino)-6-(naphthalen-1-yl)-3-(piperazin-1-yl)-5,6,7,8-tetrahydro-2,6-naphthyridine-4-carbonitrile Hydrochloride;
31. 6-(8-chloronaphthalen-1-yl)-1-(2,6-dimethylmorpholino)-3-(piperazin-1-yl)-5,6,7,8-tetrahydro-2,6-naphthyridine-4-carbonitrile Hydrochloride;
32. 3-(3-(cyanomethyl)piperazin-1-yl)-1-(2,6-dimethylmorpholino)-6-(naphthalen-1-yl)-5,6,7,8-tetrahydro-2,6-naphthyridine-4-carbonitrile Hydrochloride;
33. (S)-6-(8-chloronaphthalen-1-yl)-3-((1-methylpyrrolidin-2-yl)methoxy)-1-(piperazin-1-yl)-5,6,7,8-tetrahydro-2,6-naphthyridine-4-carbonitrile Hydrochloride;
34. (S)-6-(3-hydroxynaphthalen-1-yl)-3-((1-methylpyrrolidin-2-yl)methoxy)-1-(piperazin-1-yl)-5,6,7,8-tetrahydro-2,6-naphthyridine-4-carbonitrile Hydrochloride;
35. (S)-3-((1-methylpyrrolidin-2-yl)methoxy)-1-(piperazin-1-yl)-6-(quinazolin-4-yl)-5,6,7,8-tetrahydro-2,6-naphthyridine-4-carbonitrile Hydrochloride;
36. 6-(8-chloronaphthalen-1-yl)-1-(3-methylpiperazin-1-yl)-3-(((S)-1-methylpyrrolidin-2-yl)methoxy)-5,6,7,8-tetrahydro-2,6-naphthyridine-4-carbonitrile Hydrochloride;
37. 1-(3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
38. 1—((S)-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
39. 1—((R)-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
40. 1-(3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
41. 1—((S)-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
42. 1—((R)-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
43. 1-(3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride;
44. (S)-6-(isoquinolin-4-yl)-3-((1-methylpyrrolidin-2-yl)methoxy)-1-(piperazin-1-yl)-5,6,7,8-tetrahydro-2,6-naphthyridine-4-carbonitrile Hydrochloride;
45. (S)-3-((1-methylpyrrolidin-2-yl)methoxy)-1-(piperazin-1-yl)-6-(quinolin-8-yl)-5,6,7,8-tetrahydro-2,6-naphthyridine-4-carbonitrile Hydrochloride;
46. 6-(8-chloronaphthalen-1-yl)-1-(3-(cyanomethyl)piperazin-1-yl)-3-(((S)-1-methylpyrrolidin-2-yl)methoxy)-5, 6,7,8-tetrahydro-2,6-naphthyridine-4-carbonitrile Hydrochloride;
47. 6-(8-chloronaphthalen-1-yl)-1-((S)-3-(cyanomethyl)piperazin-1-yl)-3-(((S)-1-methylpyrrolidin-2-yl)methoxy)-5,6,7,8-tetrahydro-2,6-naphthyridine-4-carbonitrile Hydrochloride;
48. 6-(8-chloronaphthalen-1-yl)-1-((S)-3-(cyanomethyl)piperazin-1-yl)-3-(((S)-1-methylpyrrolidin-2-yl)methoxy)-5,6,7,8-tetrahydro-2,6-naphthyridine-4-carbonitrile Hydrochloride;
49. 6-benzyl-3-chloro-1-(piperazin-1-yl)-5,6,7,8-tetrahydro-2,6-naphthyridine-4-carbonitrile Hydrochloride;
50. 1-(4-aminopiperidin-1-yl)-6-benzyl-3-chloro-5,6,7,8-tetrahydro-2,6-naphthyridine-4-carbonitrile Hydrochloride;
51. 1-(4-aminopiperidin-1-yl)-6-benzyl-5,6,7,8-tetrahydro-2,6-naphthyridine-4-carbonitrile Hydrochloride;
52. 6-benzyl-1,3-di(piperazin-1-yl)-5,6,7,8-tetrahydro-2,6-naphthyridine-4-carbonitrile Hydrochloride;
53. 6-benzyl-3-hydroxy-1-(piperazin-1-yl)-5,6,7,8-tetrahydro-2,6-naphthyridine-4-carbonitrile Hydrochloride;
54. 6-benzyl-3-morpholino-1-(piperazin-1-yl)-5,6,7,8-tetrahydro-2,6-naphthyridine-4-carbonitrile Hydrochloride;
55. 6-benzyl-3-(4-methylpiperazin-1-yl)-1-(piperazin-1-yl)-5,6,7,8-tetrahydro-2,6-naphthyridine-4-carbonitrile Hydrochloride;
56. 6-benzyl-1-(3-(cyanomethyl)piperazin-1-yl)-3-(4-methylpiperazin-1-yl)-5,6,7,8-tetrahydro-2,6-naphthyridine-4-carbonitrile Hydrochloride;
57. 6-(1-naphthoyl)-1-(3-(cyanomethyl)piperazin-1-yl)-3-(4-methylpiperazin-1-yl)-5,6,7,8-tetrahydro-2,6-naphthyridine-4-carbonitrile Hydrochloride;
58. (S)-6-benzyl-1-(2-methylpiperazin-1-yl)-3-(4-methylpiperazin-1-yl)-5,6,7,8-tetrahydro-2,6-naphthyridine-4-carbonitrile Hydrochloride;
59. (R)-6-benzyl-1-(2-methylpiperazin-1-yl)-3-(4-methylpiperazin-1-yl)-5,6,7,8-tetrahydro-2,6-naphthyridine-4-carbonitrile Hydrochloride;
60. 3-(4-methylpiperazin-1-yl)-6-(naphthalen-1-yl)-1-(piperazin-1-yl)-5,6,7,8-tetrahydro-2,6-naphthyridine-4-carbonitrile Hydrochloride;
61. 6-benzyl-1-(piperazin-1-yl)-3-(piperidin-1-yl)-5,6,7,8-tetrahydro-2,6-naphthyridine-4-carbonitrile Hydrochloride;
62. 6-benzyl-3-(2-fluoropyridin-4-yl)-1-(piperazin-1-yl)-5,6,7,8-tetrahydro-2,6-naphthyridine-4-carbonitrile;
63. 6-(naphthalen-1-yl)-1-(piperazin-1-yl)-3-(piperidin-1-yl)-5,6,7,8-tetrahydro-2,6-naphthyridine-4-carbonitrile Hydrochloride;
64. 6-benzyl-3-((1-methylpyrrolidin-2-yl)methoxy)-1-(piperazin-1-yl)-5,6,7,8-tetrahydro-2,6-naphthyridine-4-carbonitrile Hydrochloride;
65. 6-(naphthalen-1-yl)-1,3-di(piperazin-1-yl)-5,6,7,8-tetrahydro-2,6-naphthyridine-4-carbonitrile Hydrochloride;
66. 6-(benzo[b]thiophen-4-yl)-1,3-di(piperazin-1-yl)-5,6,7,8-tetrahydro-2,6-naphthyridine-4-carbonitrile dihydrochloride;
67. 3-morpholino-6-(naphthalen-1-yl)-1-(piperazin-1-yl)-5,6,7,8-tetrahydro-2,6-naphthyridine-4-carbonitrile Hydrochloride;
68. 3-(4-acetylpiperazin-1-yl)-6-(naphthalen-1-yl)-1-(piperazin-1-yl)-5,6,7,8-tetrahydro-2,6-naphthyridine-4-carbonitrile Hydrochloride;
69. 3-(2,6-dimethylmorpholino)-6-(naphthalen-1-yl)-1-(piperazin-1-yl)-5,6,7,8-tetrahydro-2,6-naphthyridine-4-carbonitrile Hydrochloride;
70. 3-(4-(methylsulfonyl)piperazin-1-yl)-6-(naphthalen-1-yl)-1-(piperazin-1-yl)-5,6,7,8-tetrahydro-2,6-naphthyridine-4-carbonitrile Hydrochloride;
71. 3-(4-ethylpiperazin-1-yl)-6-(naphthalen-1-yl)-1-(piperazin-1-yl)-5,6,7,8-tetrahydro-2,6-naphthyridine-4-carbonitrile Hydrochloride;
72. (S)-3-((1-methylpyrrolidin-2-yl)methoxy)-6-(naphthalen-1-yl)-1-(piperazin-1-yl)-5,6,7,8-tetrahydro-2,6-naphthyridine-4-carbonitrile Hydrochloride;
73. 6-(naphthalen-1-yl)-1-(piperazin-1-yl)-5,6,7,8-tetrahydro-2,6-naphthyridine-4-carbonitrile Hydrochloride;
74. 6-(naphthalen-1-yl)-1-(piperazin-1-yl)-3-(4-propionylpiperazin-1-yl)-5,6,7,8-tetrahydro-2,6-naphthyridine-4-carbonitrile Hydrochloride; and
75. 6-benzyl-1-((S)-2-methylpiperazin-1-yl)-3-((1-methylpyrrolidin-2-yl)methoxy)-5,6,7,8-tetrahydro-2,6-naphthyridine-4-carbonitrile

TABLE 1
Examples

| Ex. | Structure |
|---|---|
| 1 | 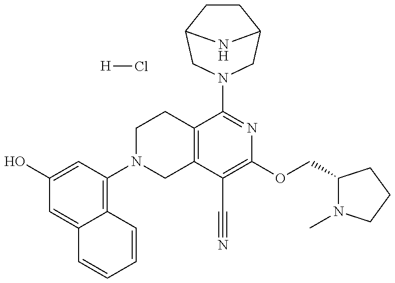 [SMILES: CN1CCC[C@H]1COc1nc(N2CC3CCC(C2)N3)c2c(c1C#N)CN(c1cc(O)cc3ccccc13)CC2 ; Cl] |
| 2 | 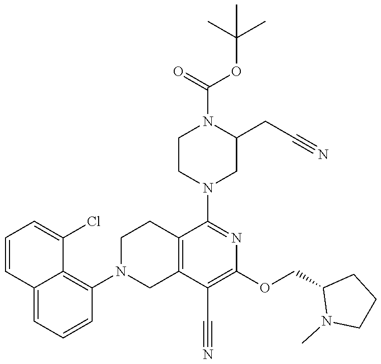 [SMILES: CN1CCC[C@H]1COc1nc(N2CCN(C(=O)OC(C)(C)C)C(CC#N)C2)c2c(c1C#N)CN(c1cccc3cccc(Cl)c13)CC2] |
| 3 | 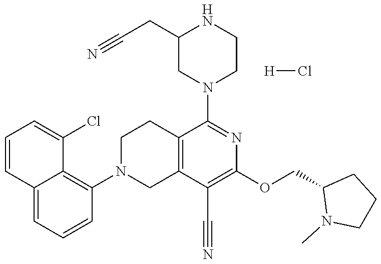 |
| 4 | 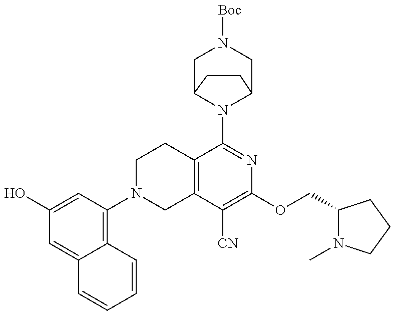 [SMILES: CN1CCC[C@H]1COc1nc(N2C3CCC2CN(C(=O)OC(C)(C)C)C3)c2c(c1C#N)CN(c1cc(O)cc3ccccc13)CC2] |
| 5 | 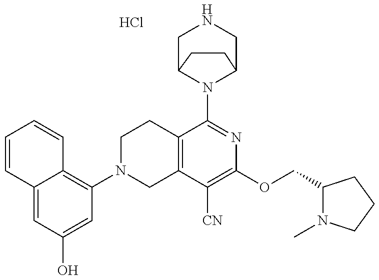 |
| 6 | 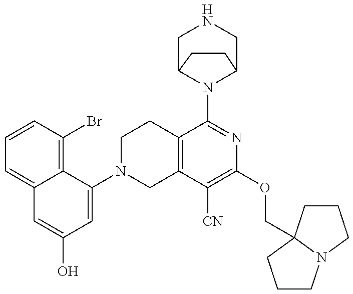 [SMILES: N#Cc1c(OCC23CCCN2CCC3)nc(N2C3CCC2CNC3)c2c1CN(c1cc(O)cc3cccc(Br)c13)CC2] |
| 7 | 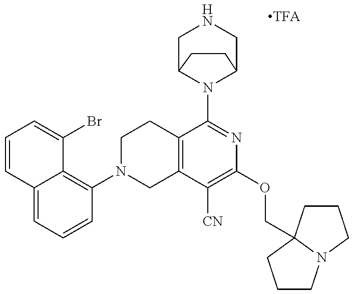 [SMILES: N#Cc1c(OCC23CCCN2CCC3)nc(N2C3CCC2CNC3)c2c1CN(c1cccc3cccc(Br)c13)CC2] |
| 8 | 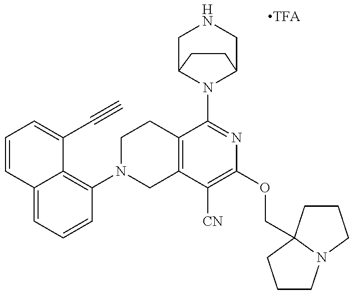 [SMILES: C#Cc1cccc2cccc(N3CCc4c(N5C6CCC5CNC6)nc(OCC56CCCN5CCC6)c(C#N)c4C3)c12] |
| 9 | 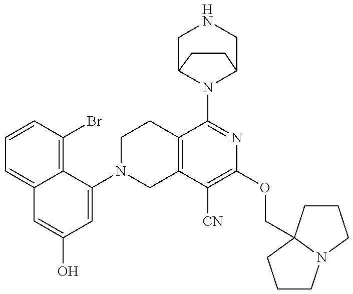 [SMILES: N#Cc1c(OCC23CCCN2CCC3)nc(N2C3CCC2CNC3)c2c1CN(c1cc(O)cc3cccc(Br)c13)CC2] |
| 10 | 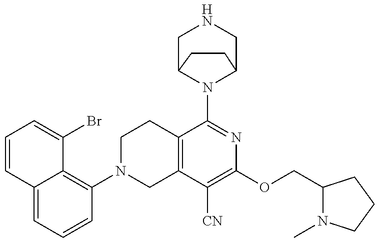 [SMILES: CN1CCCC1COc1nc(N2C3CCC2CNC3)c2c(c1C#N)CN(c1cccc3cccc(Br)c13)CC2] |
| 11 | 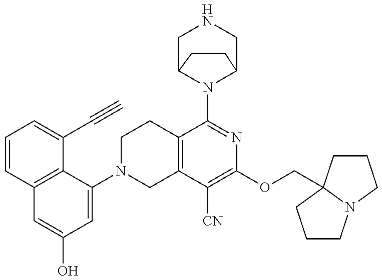 [SMILES: C#Cc1cccc2cc(O)cc(N3CCc4c(N5C6CCC5CNC6)nc(OCC56CCCN5CCC6)c(C#N)c4C3)c12] |
| 12 | 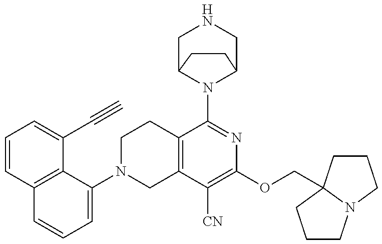 [SMILES: C#Cc1cccc2cccc(N3CCc4c(N5C6CCC5CNC6)nc(OCC56CCCN5CCC6)c(C#N)c4C3)c12] |
| 13 | 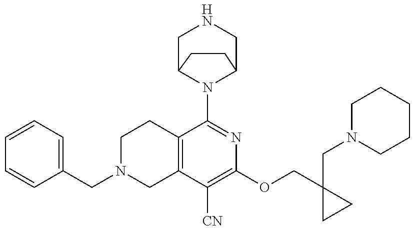 [SMILES: N#Cc1c(OCC2(CN3CCCCC3)CC2)nc(N2C3CCC2CNC3)c2c1CN(Cc1ccccc1)CC2] |
| 14 | 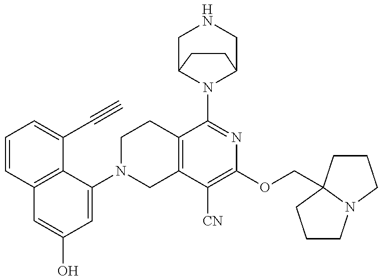 [SMILES: C#Cc1cccc2cc(O)cc(N3CCc4c(N5C6CCC5CNC6)nc(OCC56CCCN5CCC6)c(C#N)c4C3)c12] |
| 15 | 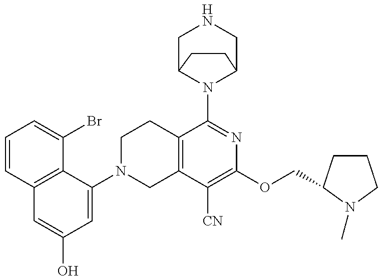 [SMILES: CN1CCC[C@H]1COc1nc(N2C3CCC2CNC3)c2c(c1C#N)CN(c1cc(O)cc3cccc(Br)c13)CC2] |
| 16 | 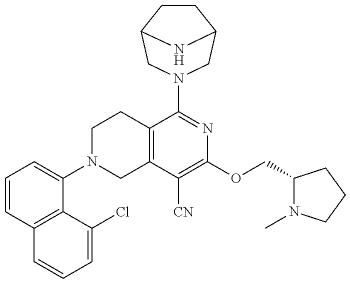 [SMILES: CN1CCC[C@H]1COc1nc(N2CC3CCC(C2)N3)c2c(c1C#N)CN(c1cccc3cccc(Cl)c13)CC2] |
| 17 | 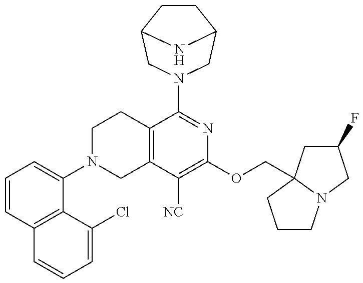 [SMILES: N#Cc1c(OCC23CCCN2C[C@H](F)C3)nc(N2CC3CCC(C2)N3)c2c1CN(c1cccc3cccc(Cl)c13)CC2] |
| 18 | 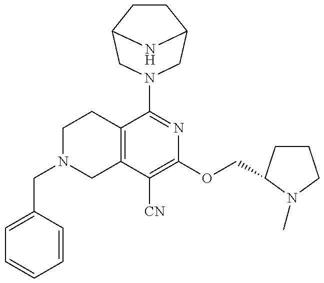 [SMILES: CN1CCC[C@H]1COc1nc(N2CC3CCC(C2)N3)c2c(c1C#N)CN(Cc1ccccc1)CC2] |
| 19 | 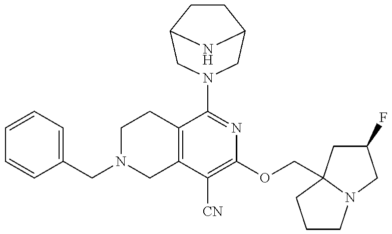 [SMILES: N#Cc1c(OCC23CCCN2C[C@H](F)C3)nc(N2CC3CCC(C2)N3)c2c1CN(Cc1ccccc1)CC2] |
| 20 | 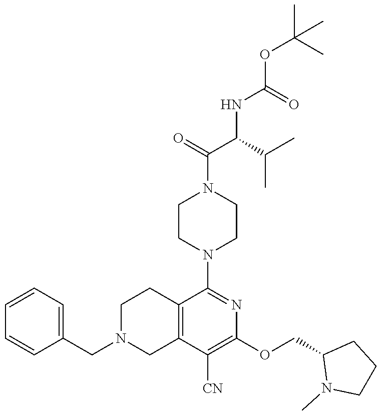 [SMILES: CC(C)[C@@H](NC(=O)OC(C)(C)C)C(=O)N1CCN(c2nc(OC[C@@H]3CCCN3C)c(C#N)c3c2CCN(Cc2ccccc2)C3)CC1] |
| 21 | 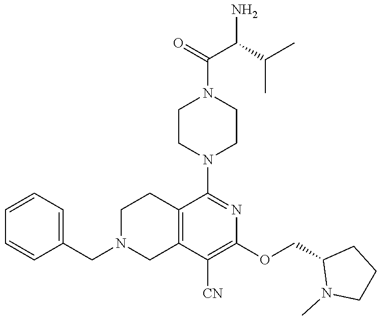 [SMILES: CC(C)[C@@H](N)C(=O)N1CCN(c2nc(OC[C@@H]3CCCN3C)c(C#N)c3c2CCN(Cc2ccccc2)C3)CC1] |
| 22 | 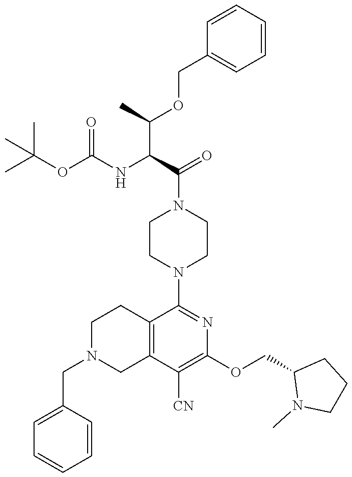 [SMILES: C[C@@H](OCc1ccccc1)[C@H](NC(=O)OC(C)(C)C)C(=O)N1CCN(c2nc(OC[C@@H]3CCCN3C)c(C#N)c3c2CCN(Cc2ccccc2)C3)CC1] |
| 23 | 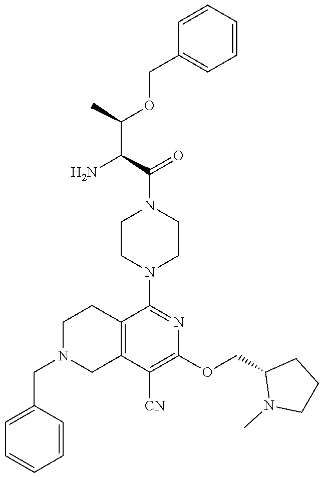 [SMILES: C[C@@H](OCc1ccccc1)[C@H](N)C(=O)N1CCN(c2nc(OC[C@@H]3CCCN3C)c(C#N)c3c2CCN(Cc2ccccc2)C3)CC1] |
| 24 | 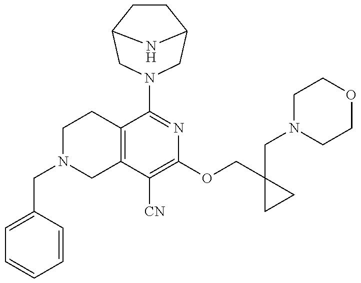 [SMILES: N#Cc1c(OCC2(CN3CCOCC3)CC2)nc(N2CC3CCC(C2)N3)c2c1CN(Cc1ccccc1)CC2] |
| 25 | 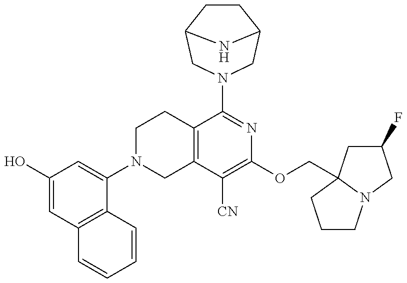 [SMILES: N#Cc1c(OCC23CCCN2C[C@H](F)C3)nc(N2CC3CCC(C2)N3)c2c1CN(c1cc(O)cc3ccccc13)CC2] |
| 26 | 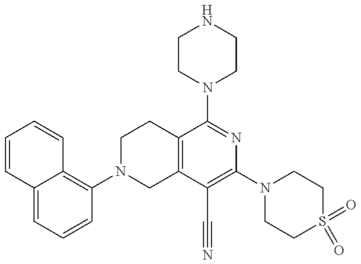 [SMILES: N#Cc1c(N2CCS(=O)(=O)CC2)nc(N2CCNCC2)c2c1CN(c1cccc3ccccc13)CC2] |
| 27 | 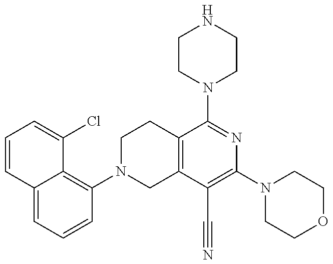 [SMILES: N#Cc1c(N2CCOCC2)nc(N2CCNCC2)c2c1CN(c1cccc3cccc(Cl)c13)CC2] |
| 28 | 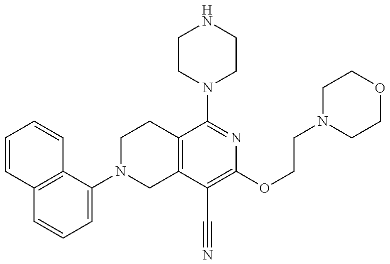 [SMILES: N#Cc1c(OCCN2CCOCC2)nc(N2CCNCC2)c2c1CN(c1cccc3ccccc13)CC2] |
| 29 | 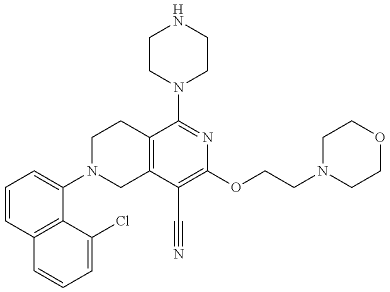 [SMILES: N#Cc1c(OCCN2CCOCC2)nc(N2CCNCC2)c2c1CN(c1cccc3cccc(Cl)c13)CC2] |
| 30 | 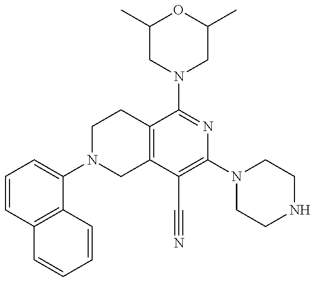 [SMILES: CC1CN(c2nc(N3CCNCC3)c(C#N)c3c2CCN(c2cccc4ccccc24)C3)CC(C)O1] |
| 31 | 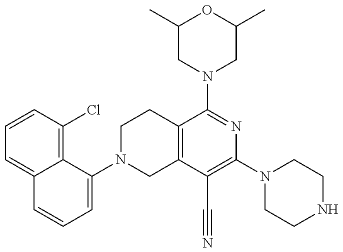 [SMILES: CC1CN(c2nc(N3CCNCC3)c(C#N)c3c2CCN(c2cccc4cccc(Cl)c24)C3)CC(C)O1] |
| 32 | 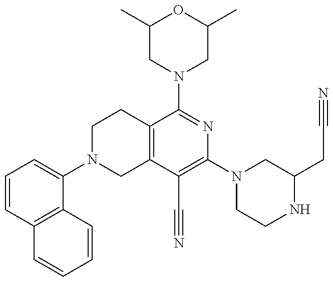 [SMILES: CC1CN(c2nc(N3CCNC(CC#N)C3)c(C#N)c3c2CCN(c2cccc4ccccc24)C3)CC(C)O1] |
| 33 | 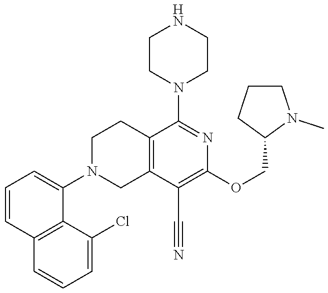 [SMILES: CN1CCC[C@H]1COc1nc(N2CCNCC2)c2c(c1C#N)CN(c1cccc3cccc(Cl)c13)CC2] |
| 34 | 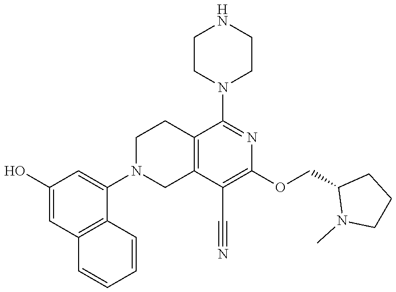 [SMILES: CN1CCC[C@H]1COc1nc(N2CCNCC2)c2c(c1C#N)CN(c1cc(O)cc3ccccc13)CC2] |
| 35 | 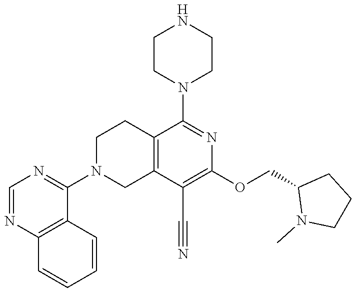 [SMILES: CN1CCC[C@H]1COc1nc(N2CCNCC2)c2c(c1C#N)CN(c1ncnc3ccccc13)CC2] |
| 36 | 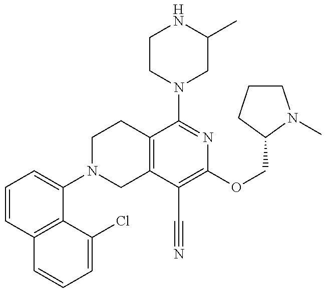 [SMILES: CC1CN(c2nc(OC[C@@H]3CCCN3C)c(C#N)c3c2CCN(c2cccc4cccc(Cl)c24)C3)CCN1] |
| 37 | 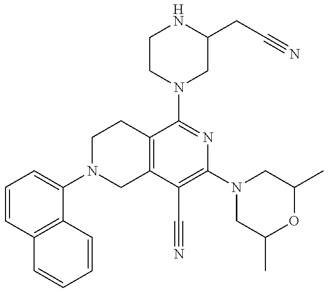 [SMILES: CC1CN(c2nc(N3CCNC(CC#N)C3)c3c(c2C#N)CN(c2cccc4ccccc24)CC3)CC(C)O1] |
| 38 | 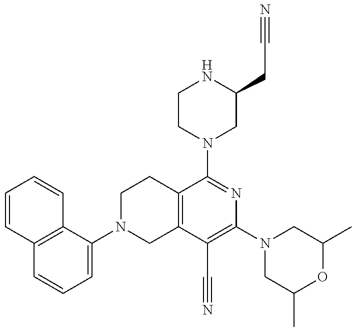 [SMILES: CC1CN(c2nc(N3CCN[C@@H](CC#N)C3)c3c(c2C#N)CN(c2cccc4ccccc24)CC3)CC(C)O1] |
| 39 | 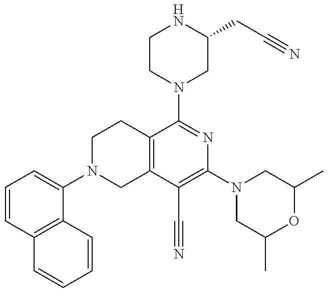 [SMILES: CC1CN(c2nc(N3CCN[C@H](CC#N)C3)c3c(c2C#N)CN(c2cccc4ccccc24)CC3)CC(C)O1] |
| 40 | 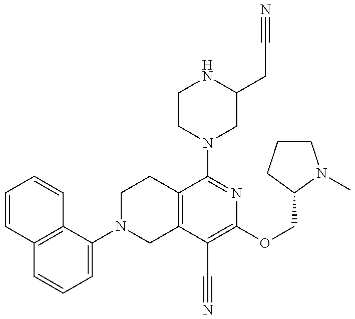 [SMILES: CN1CCC[C@H]1COc1nc(N2CCNC(CC#N)C2)c2c(c1C#N)CN(c1cccc3ccccc13)CC2] |
| 41 | 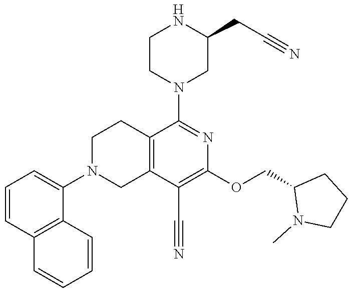 [SMILES: CN1CCC[C@H]1COc1nc(N2CCN[C@@H](CC#N)C2)c2c(c1C#N)CN(c1cccc3ccccc13)CC2] |
| 42 | 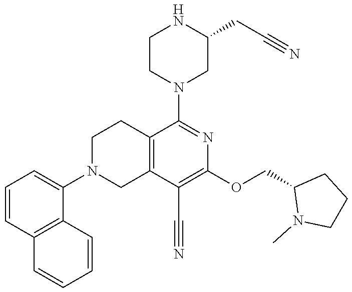 [SMILES: CN1CCC[C@H]1COc1nc(N2CCN[C@H](CC#N)C2)c2c(c1C#N)CN(c1cccc3ccccc13)CC2] |
| 43 | 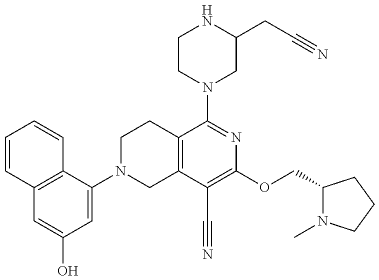 [SMILES: CN1CCC[C@H]1COc1nc(N2CCNC(CC#N)C2)c2c(c1C#N)CN(c1cc(O)cc3ccccc13)CC2] |
| 44 | 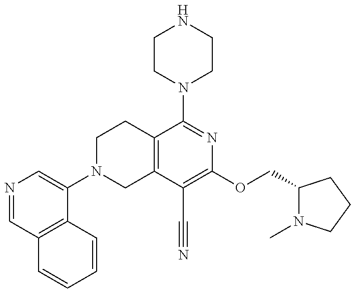 [SMILES: CN1CCC[C@H]1COc1nc(N2CCNCC2)c2c(c1C#N)CN(c1cncc3ccccc13)CC2] |
| 45 | 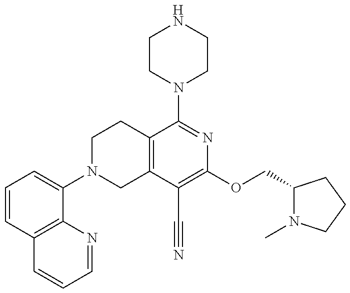 [SMILES: CN1CCC[C@H]1COc1nc(N2CCNCC2)c2c(c1C#N)CN(c1cccc3cccnc13)CC2] |
| 46 | 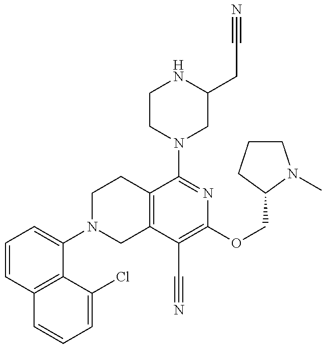 [SMILES: CN1CCC[C@H]1COc1nc(N2CCNC(CC#N)C2)c2c(c1C#N)CN(c1cccc3cccc(Cl)c13)CC2] |
| 47 | 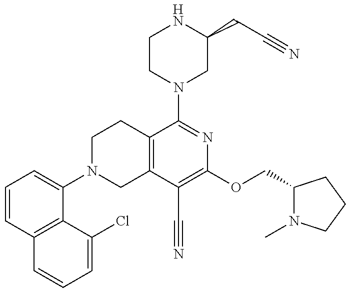 [SMILES: CN1CCC[C@H]1COc1nc(N2CCN[C@@H](CC#N)C2)c2c(c1C#N)CN(c1cccc3cccc(Cl)c13)CC2] |
| 48 | 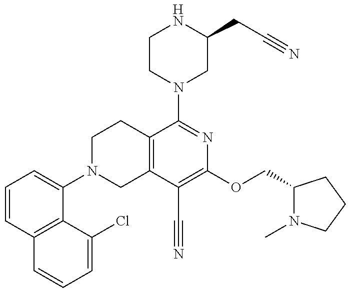 [SMILES: CN1CCC[C@H]1COc1nc(N2CCN[C@@H](CC#N)C2)c2c(c1C#N)CN(c1cccc3cccc(Cl)c13)CC2] |
| 49 | 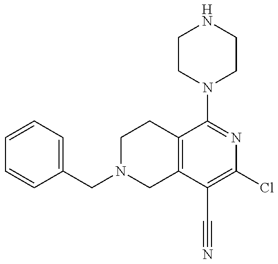 [SMILES: N#Cc1c(Cl)nc(N2CCNCC2)c2c1CN(Cc1ccccc1)CC2] |
| 50 | 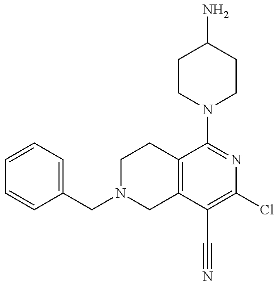 [SMILES: N#Cc1c(Cl)nc(N2CCC(N)CC2)c2c1CN(Cc1ccccc1)CC2] |
| 51 | 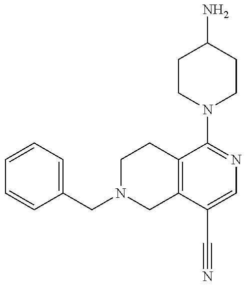 [SMILES: N#Cc1cnc(N2CCC(N)CC2)c2c1CN(Cc1ccccc1)CC2] |
| 52 | 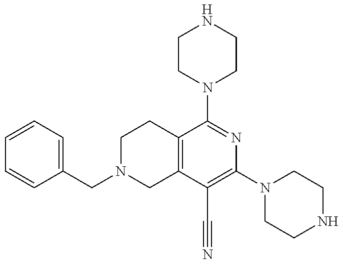 [SMILES: N#Cc1c(N2CCNCC2)nc(N2CCNCC2)c2c1CN(Cc1ccccc1)CC2] |
| 53 | 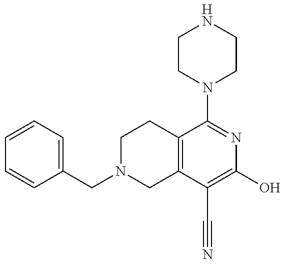 [SMILES: N#Cc1c(O)nc(N2CCNCC2)c2c1CN(Cc1ccccc1)CC2] |
| 54 | 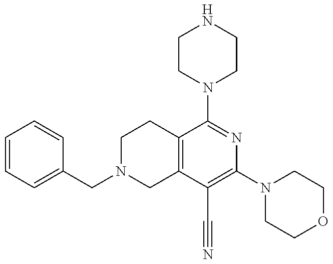 [SMILES: N#Cc1c(N2CCOCC2)nc(N2CCNCC2)c2c1CN(Cc1ccccc1)CC2] |
| 55 | 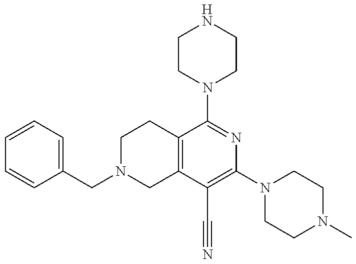 [SMILES: CN1CCN(c2nc(N3CCNCC3)c3c(c2C#N)CN(Cc2ccccc2)CC3)CC1] |
| 56 | 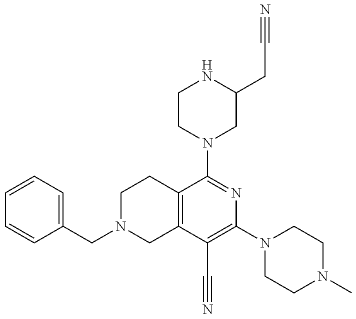 [SMILES: CN1CCN(c2nc(N3CCNC(CC#N)C3)c3c(c2C#N)CN(Cc2ccccc2)CC3)CC1] |
| 57 | 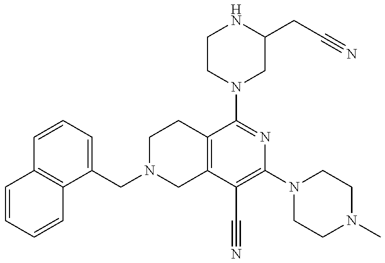 [SMILES: CN1CCN(c2nc(N3CCNC(CC#N)C3)c3c(c2C#N)CN(Cc2cccc4ccccc24)CC3)CC1] |
| 58 | 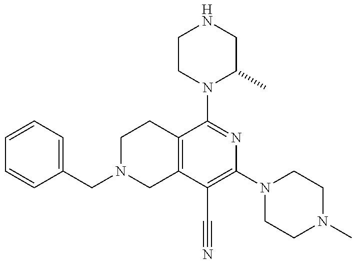 [SMILES: C[C@H]1CNCCN1c1nc(N2CCN(C)CC2)c(C#N)c2c1CCN(Cc1ccccc1)C2] |
| 59 | 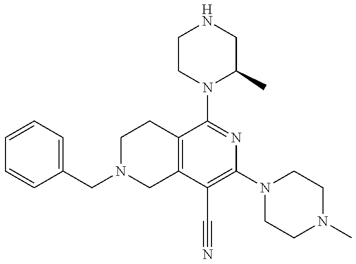 [SMILES: C[C@@H]1CNCCN1c1nc(N2CCN(C)CC2)c(C#N)c2c1CCN(Cc1ccccc1)C2] |
| 60 | 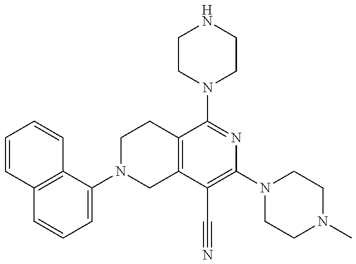 [SMILES: CN1CCN(c2nc(N3CCNCC3)c3c(c2C#N)CN(c2cccc4ccccc24)CC3)CC1] |
| 61 | 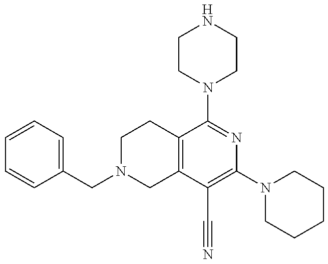 [SMILES: N#Cc1c(N2CCCCC2)nc(N2CCNCC2)c2c1CN(Cc1ccccc1)CC2] |
| 62 | 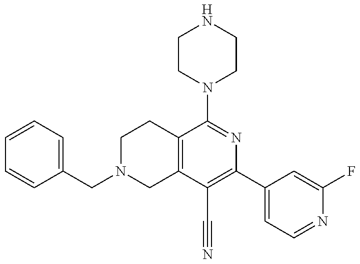 [SMILES: N#Cc1c(-c2ccnc(F)c2)nc(N2CCNCC2)c2c1CN(Cc1ccccc1)CC2] |
| 63 | 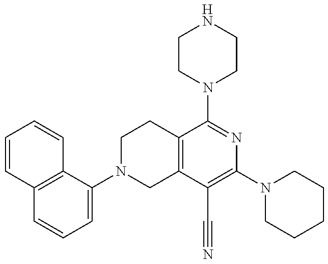 [SMILES: N#Cc1c(N2CCCCC2)nc(N2CCNCC2)c2c1CN(c1cccc3ccccc13)CC2] |
| 64 | 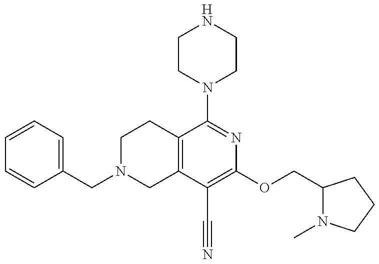 [SMILES: CN1CCCC1COc1nc(N2CCNCC2)c2c(c1C#N)CN(Cc1ccccc1)CC2] |
| 65 | 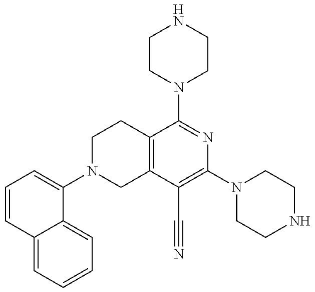 [SMILES: N#Cc1c(N2CCNCC2)nc(N2CCNCC2)c2c1CN(c1cccc3ccccc13)CC2] |
| 66 | 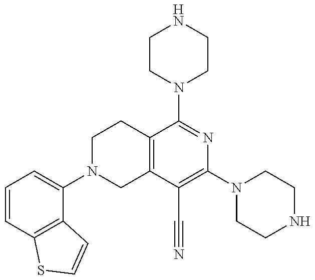 [SMILES: N#Cc1c(N2CCNCC2)nc(N2CCNCC2)c2c1CN(c1cccc3sccc13)CC2] |
| 67 | 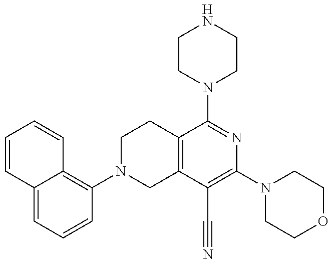 [SMILES: N#Cc1c(N2CCOCC2)nc(N2CCNCC2)c2c1CN(c1cccc3ccccc13)CC2] |
| 68 | 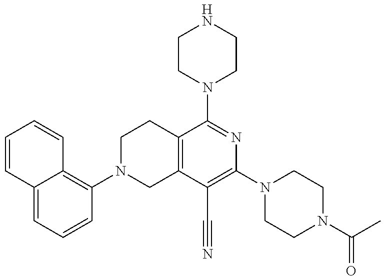 [SMILES: CC(=O)N1CCN(c2nc(N3CCNCC3)c3c(c2C#N)CN(c2cccc4ccccc24)CC3)CC1] |
| 69 | 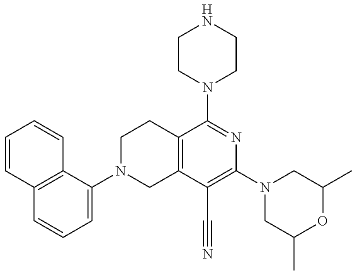 [SMILES: CC1CN(c2nc(N3CCNCC3)c3c(c2C#N)CN(c2cccc4ccccc24)CC3)CC(C)O1] |
| 70 | 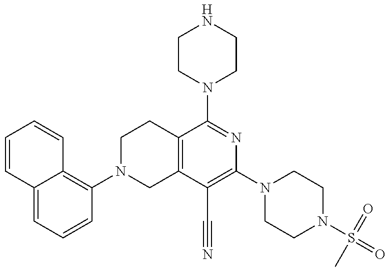 [SMILES: CS(=O)(=O)N1CCN(c2nc(N3CCNCC3)c3c(c2C#N)CN(c2cccc4ccccc24)CC3)CC1] |
| 71 | 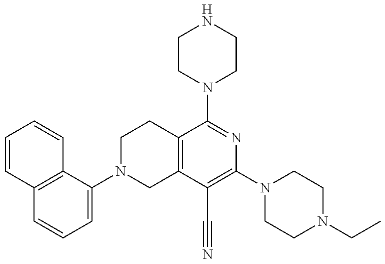 [SMILES: CCN1CCN(c2nc(N3CCNCC3)c3c(c2C#N)CN(c2cccc4ccccc24)CC3)CC1] |
| 72 | 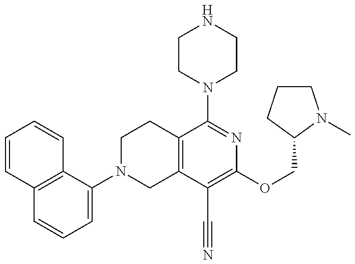 [SMILES: CN1CCC[C@H]1COc1nc(N2CCNCC2)c2c(c1C#N)CN(c1cccc3ccccc13)CC2] |
| 73 | 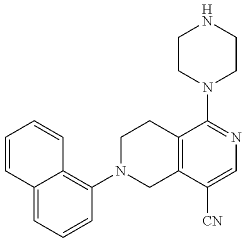 [SMILES: N#Cc1cnc(N2CCNCC2)c2c1CN(c1cccc3ccccc13)CC2] |
| 74 | 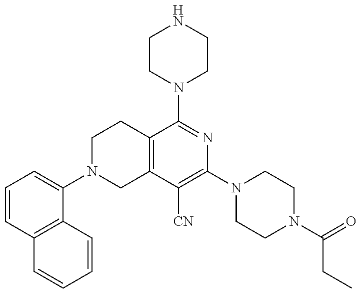 [SMILES: CCC(=O)N1CCN(c2nc(N3CCNCC3)c3c(c2C#N)CN(c2cccc4ccccc24)CC3)CC1] |
| 75 | 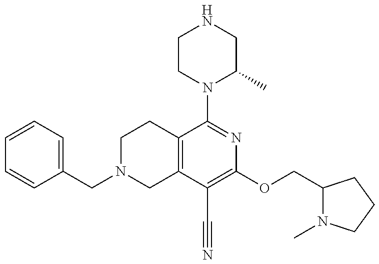 [SMILES: C[C@H]1CNCCN1c1nc(OCC2CCCN2C)c(C#N)c2c1CCN(Cc1ccccc1)C2] |

The non-limiting representative compounds of the present invention as listed herein below, and pharmaceutically acceptable salts thereof are

TABLE 2

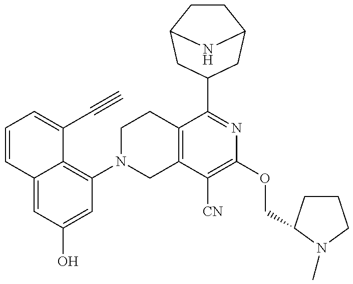 [SMILES: C#Cc1cccc2cc(O)cc(N3CCc4c(C5CC6CCC(C5)N6)nc(OC[C@@H]5CCCN5C)c(C#N)c4C3)c12]

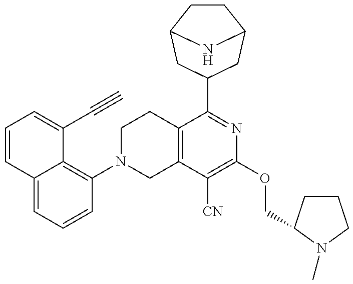 [SMILES: C#Cc1cccc2cccc(N3CCc4c(C5CC6CCC(C5)N6)nc(OC[C@@H]5CCCN5C)c(C#N)c4C3)c12]

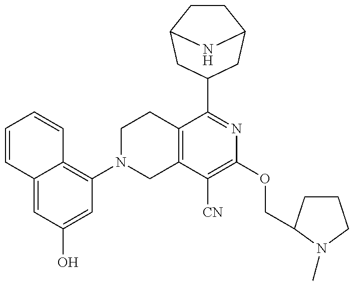 [SMILES: CN1CCCC1COc1nc(C2CC3CCC(C2)N3)c2c(c1C#N)CN(c1cc(O)cc3ccccc13)CC2]

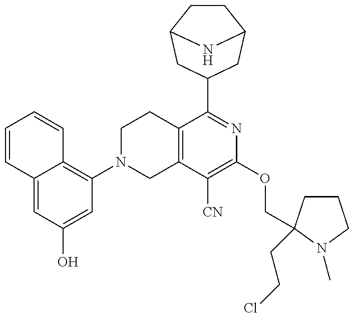 [SMILES: CN1CCCC1(CCCl)COc1nc(C2CC3CCC(C2)N3)c2c(c1C#N)CN(c1cc(O)cc3ccccc13)CC2]

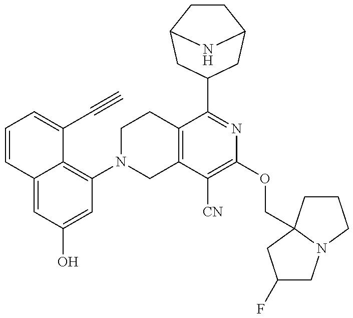 [SMILES: C#Cc1cccc2cc(O)cc(N3CCc4c(C5CC6CCC(C5)N6)nc(OCC56CCCN5CC(F)C6)c(C#N)c4C3)c12]

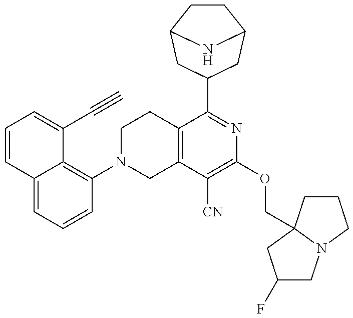 [SMILES: C#Cc1cccc2cccc(N3CCc4c(C5CC6CCC(C5)N6)nc(OCC56CCCN5CC(F)C6)c(C#N)c4C3)c12]

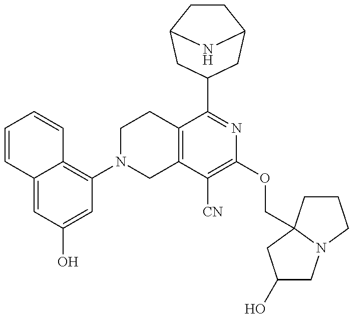 [SMILES: N#Cc1c(OCC23CCCN2CC(O)C3)nc(C2CC3CCC(C2)N3)c2c1CN(c1cc(O)cc3ccccc13)CC2]

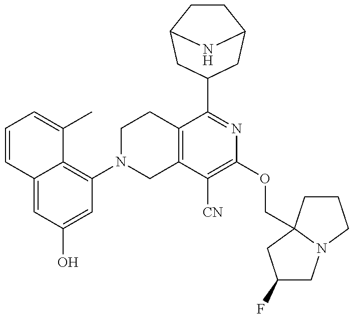 [SMILES: Cc1cccc2cc(O)cc(N3CCc4c(C5CC6CCC(C5)N6)nc(OCC56CCCN5C[C@@H](F)C6)c(C#N)c4C3)c12]

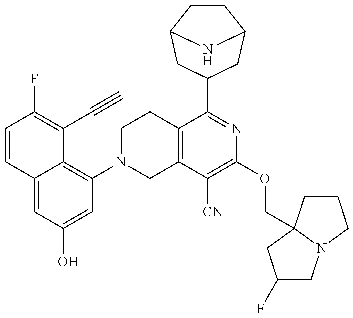 [SMILES: C#Cc1c(F)ccc2cc(O)cc(N3CCc4c(C5CC6CCC(C5)N6)nc(OCC56CCCN5CC(F)C6)c(C#N)c4C3)c12]

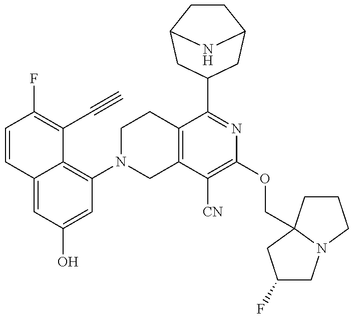 [SMILES: C#Cc1c(F)ccc2cc(O)cc(N3CCc4c(C5CC6CCC(C5)N6)nc(OCC56CCCN5C[C@H](F)C6)c(C#N)c4C3)c12]

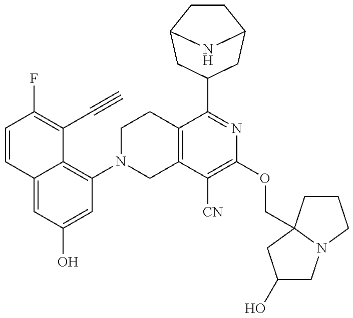 [SMILES: C#Cc1c(F)ccc2cc(O)cc(N3CCc4c(C5CC6CCC(C5)N6)nc(OCC56CCCN5CC(O)C6)c(C#N)c4C3)c12]

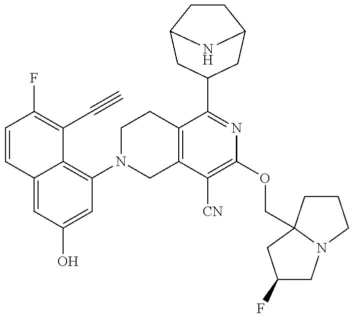 [SMILES: C#Cc1c(F)ccc2cc(O)cc(N3CCc4c(C5CC6CCC(C5)N6)nc(OCC56CCCN5C[C@@H](F)C6)c(C#N)c4C3)c12]

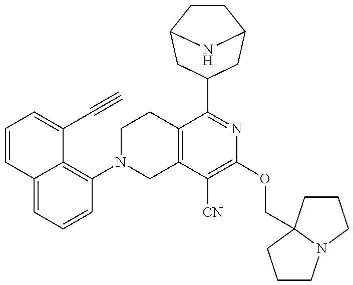 [SMILES: C#Cc1cccc2cccc(N3CCc4c(C5CC6CCC(C5)N6)nc(OCC56CCCN5CCC6)c(C#N)c4C3)c12]

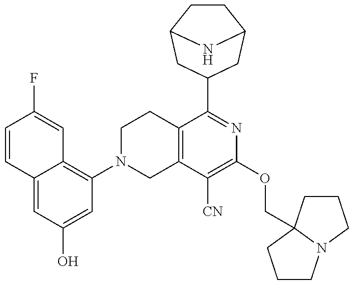 [SMILES: N#Cc1c(OCC23CCCN2CCC3)nc(C2CC3CCC(C2)N3)c2c1CN(c1cc(O)cc3ccc(F)cc13)CC2]

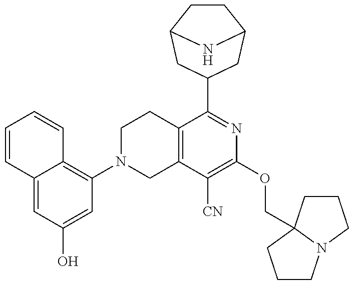 [SMILES: N#Cc1c(OCC23CCCN2CCC3)nc(C2CC3CCC(C2)N3)c2c1CN(c1cc(O)cc3ccccc13)CC2]

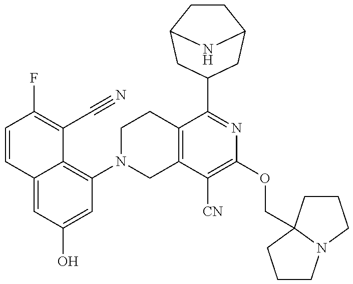 [SMILES: N#Cc1c(OCC23CCCN2CCC3)nc(C2CC3CCC(C2)N3)c2c1CN(c1cc(O)cc3ccc(F)c(C#N)c13)CC2]

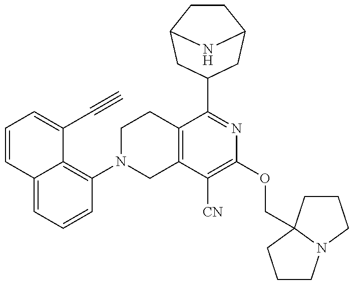 [SMILES: C#Cc1cccc2cccc(N3CCc4c(C5CC6CCC(C5)N6)nc(OCC56CCCN5CCC6)c(C#N)c4C3)c12]

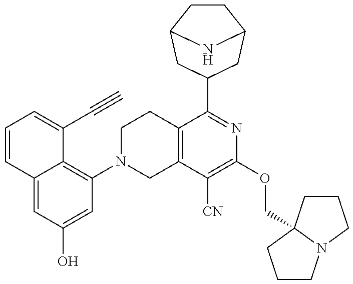

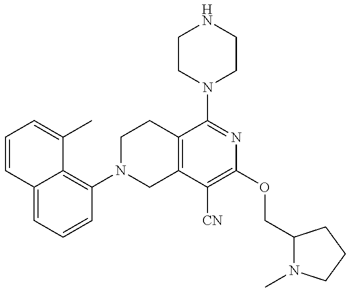 [SMILES: Cc1cccc2cccc(N3CCc4c(N5CCNCC5)nc(OCC5CCCN5C)c(C#N)c4C3)c12]

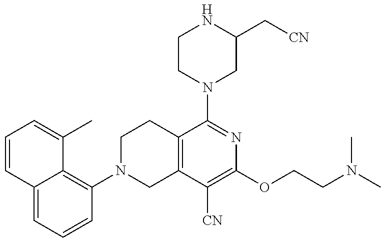 [SMILES: Cc1cccc2cccc(N3CCc4c(N5CCNC(CC#N)C5)nc(OCCN(C)C)c(C#N)c4C3)c12]

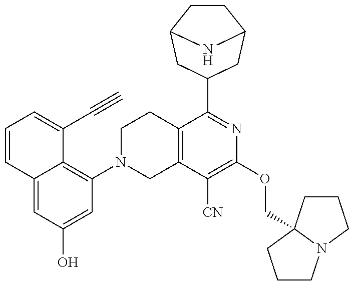

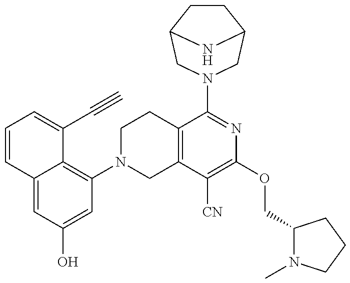 [SMILES: C#Cc1cccc2cc(O)cc(N3CCc4c(N5CC6CCC(C5)N6)nc(OC[C@@H]5CCCN5C)c(C#N)c4C3)c12]

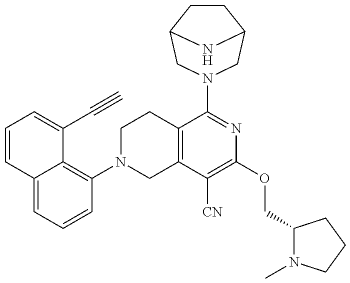 [SMILES: C#Cc1cccc2cccc(N3CCc4c(N5CC6CCC(C5)N6)nc(OC[C@@H]5CCCN5C)c(C#N)c4C3)c12]

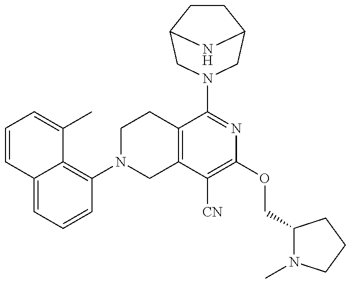 [SMILES: Cc1cccc2cccc(N3CCc4c(N5CC6CCC(C5)N6)nc(OC[C@@H]5CCCN5C)c(C#N)c4C3)c12]

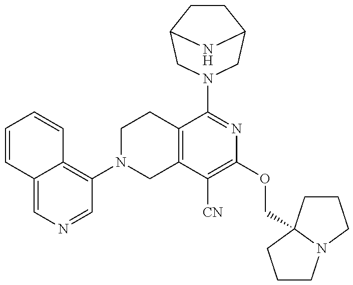

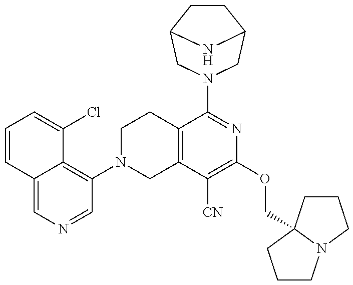

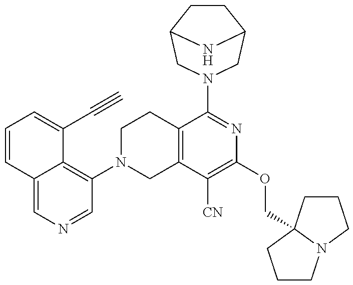

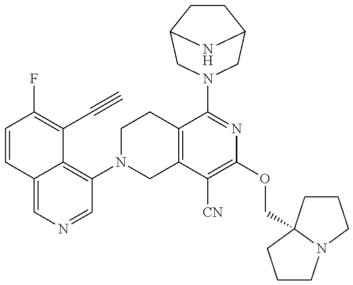

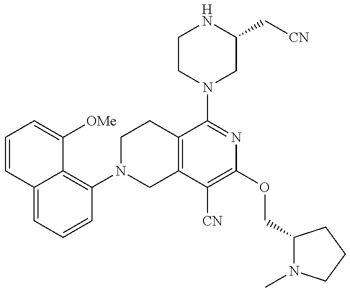 [SMILES: COc1cccc2cccc(N3CCc4c(N5CCN[C@H](CC#N)C5)nc(OC[C@@H]5CCCN5C)c(C#N)c4C3)c12]

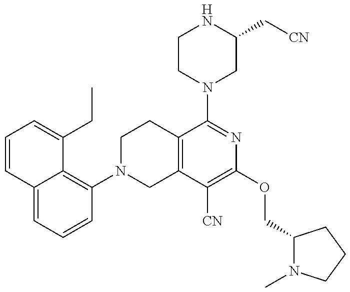 [SMILES: CCc1cccc2cccc(N3CCc4c(N5CCN[C@H](CC#N)C5)nc(OC[C@@H]5CCCN5C)c(C#N)c4C3)c12]

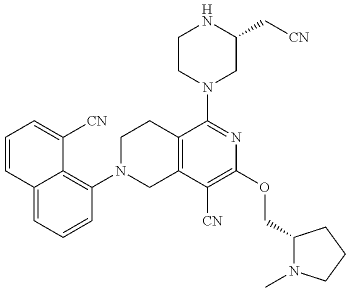 [SMILES: CN1CCC[C@H]1COc1nc(N2CCN[C@H](CC#N)C2)c2c(c1C#N)CN(c1cccc3cccc(C#N)c13)CC2]

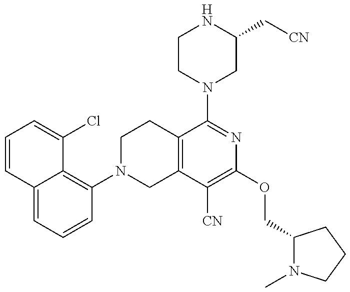 [SMILES: CN1CCC[C@H]1COc1nc(N2CCN[C@H](CC#N)C2)c2c(c1C#N)CN(c1cccc3cccc(Cl)c13)CC2]

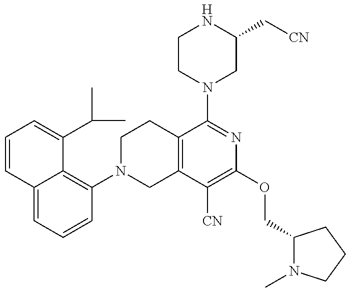 [SMILES: CC(C)c1cccc2cccc(N3CCc4c(N5CCN[C@H](CC#N)C5)nc(OC[C@@H]5CCCN5C)c(C#N)c4C3)c12]

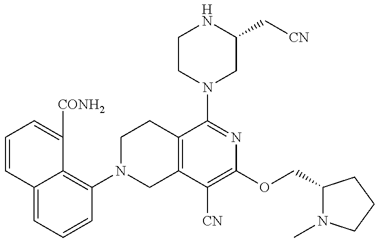 [SMILES: CN1CCC[C@H]1COc1nc(N2CCN[C@H](CC#N)C2)c2c(c1C#N)CN(c1cccc3cccc(C(N)=O)c13)CC2]

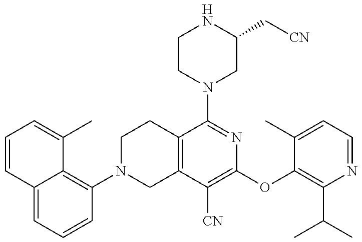 [SMILES: Cc1ccnc(C(C)C)c1Oc1nc(N2CCN[C@H](CC#N)C2)c2c(c1C#N)CN(c1cccc3cccc(C)c13)CC2]

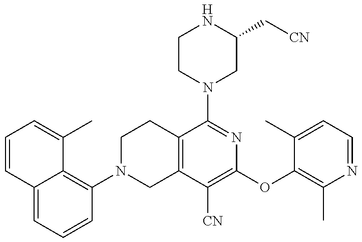 [SMILES: Cc1ccnc(C)c1Oc1nc(N2CCN[C@H](CC#N)C2)c2c(c1C#N)CN(c1cccc3cccc(C)c13)CC2]

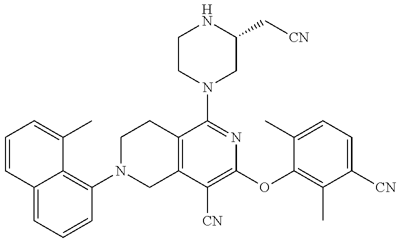 [SMILES: Cc1ccc(C#N)c(C)c1Oc1nc(N2CCN[C@H](CC#N)C2)c2c(c1C#N)CN(c1cccc3cccc(C)c13)CC2]

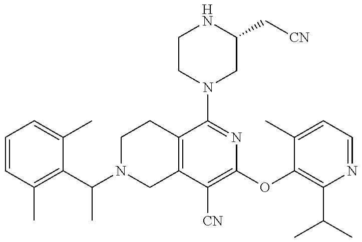 [SMILES: Cc1ccnc(C(C)C)c1Oc1nc(N2CCN[C@H](CC#N)C2)c2c(c1C#N)CN(C(C)c1c(C)cccc1C)CC2]

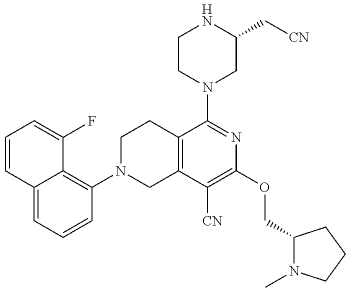 [SMILES: CN1CCC[C@H]1COc1nc(N2CCN[C@H](CC#N)C2)c2c(c1C#N)CN(c1cccc3cccc(F)c13)CC2]

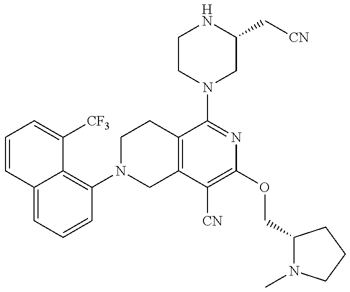 [SMILES: CN1CCC[C@H]1COc1nc(N2CCN[C@H](CC#N)C2)c2c(c1C#N)CN(c1cccc3cccc(C(F)(F)F)c13)CC2]

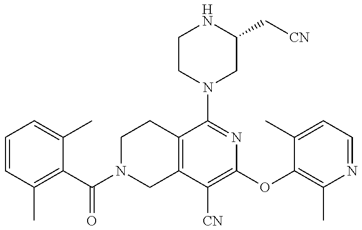 [SMILES: Cc1ccnc(C)c1Oc1nc(N2CCN[C@H](CC#N)C2)c2c(c1C#N)CN(C(=O)c1c(C)cccc1C)CC2]

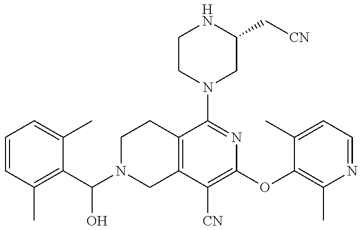 [SMILES: Cc1ccnc(C)c1Oc1nc(N2CCN[C@H](CC#N)C2)c2c(c1C#N)CN(C(O)c1c(C)cccc1C)CC2]

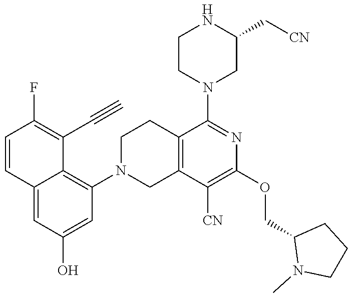 [SMILES: C#Cc1c(F)ccc2cc(O)cc(N3CCc4c(N5CCN[C@H](CC#N)C5)nc(OC[C@@H]5CCCN5C)c(C#N)c4C3)c12]

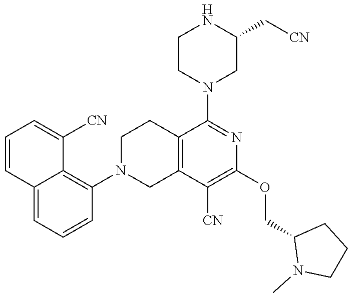 [SMILES: CN1CCC[C@H]1COc1nc(N2CCN[C@H](CC#N)C2)c2c(c1C#N)CN(c1cccc3cccc(C#N)c13)CC2]

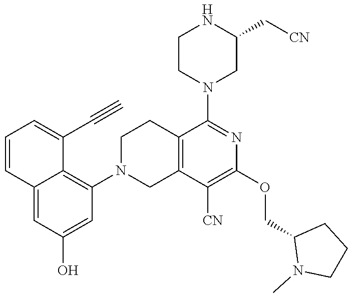 [SMILES: C#Cc1cccc2cc(O)cc(N3CCc4c(N5CCN[C@H](CC#N)C5)nc(OC[C@@H]5CCCN5C)c(C#N)c4C3)c12]

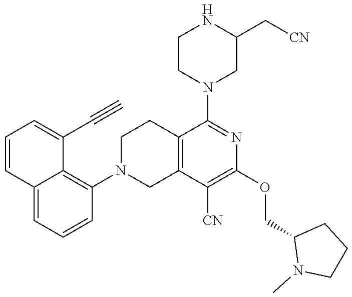 [SMILES: C#Cc1cccc2cccc(N3CCc4c(N5CCNC(CC#N)C5)nc(OC[C@@H]5CCCN5C)c(C#N)c4C3)c12]

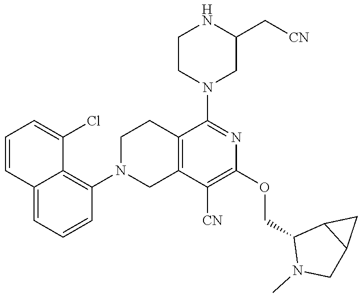 [SMILES: CN1CC2CC2[C@H]1COc1nc(N2CCNC(CC#N)C2)c2c(c1C#N)CN(c1cccc3cccc(Cl)c13)CC2]

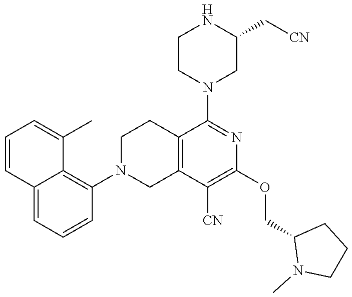 [SMILES: Cc1cccc2cccc(N3CCc4c(N5CCN[C@H](CC#N)C5)nc(OC[C@@H]5CCCN5C)c(C#N)c4C3)c12]

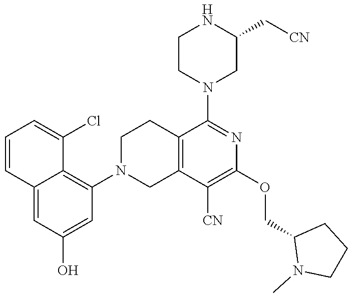 [SMILES: CN1CCC[C@H]1COc1nc(N2CCN[C@H](CC#N)C2)c2c(c1C#N)CN(c1cc(O)cc3cccc(Cl)c13)CC2]

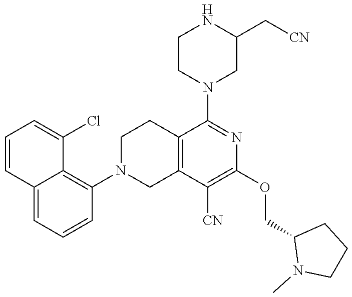 [SMILES: CN1CCC[C@H]1COc1nc(N2CCNC(CC#N)C2)c2c(c1C#N)CN(c1cccc3cccc(Cl)c13)CC2]

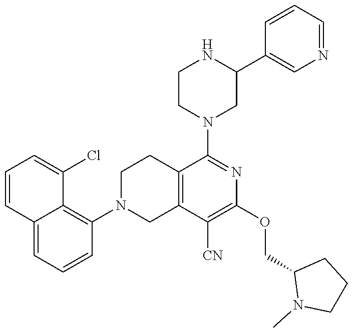 [SMILES: CN1CCC[C@H]1COc1nc(N2CCNC(c3cccnc3)C2)c2c(c1C#N)CN(c1cccc3cccc(Cl)c13)CC2]

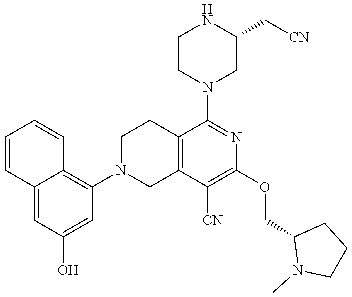 [SMILES: CN1CCC[C@H]1COc1nc(N2CCN[C@H](CC#N)C2)c2c(c1C#N)CN(c1cc(O)cc3ccccc13)CC2]

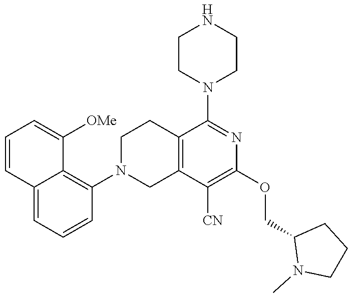 [SMILES: COc1cccc2cccc(N3CCc4c(N5CCNCC5)nc(OC[C@@H]5CCCN5C)c(C#N)c4C3)c12]

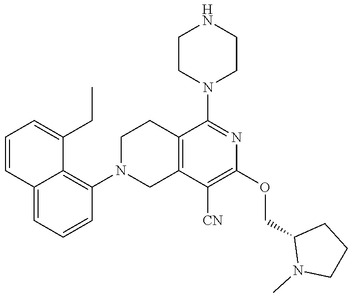 [SMILES: CCc1cccc2cccc(N3CCc4c(N5CCNCC5)nc(OC[C@@H]5CCCN5C)c(C#N)c4C3)c12]

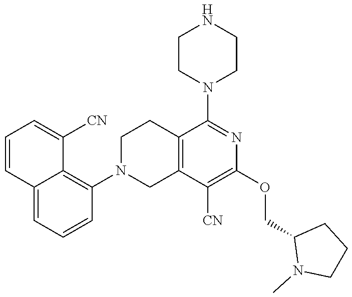 [SMILES: CN1CCC[C@H]1COc1nc(N2CCNCC2)c2c(c1C#N)CN(c1cccc3cccc(C#N)c13)CC2]

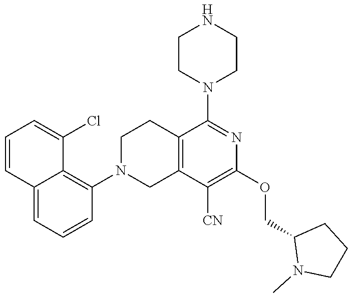 [SMILES: CN1CCC[C@H]1COc1nc(N2CCNCC2)c2c(c1C#N)CN(c1cccc3cccc(Cl)c13)CC2]

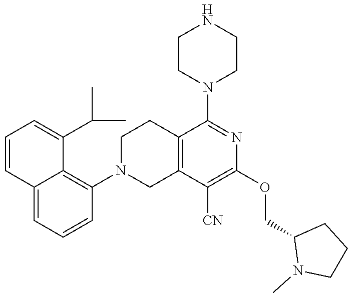 [SMILES: CC(C)c1cccc2cccc(N3CCc4c(N5CCNCC5)nc(OC[C@@H]5CCCN5C)c(C#N)c4C3)c12]

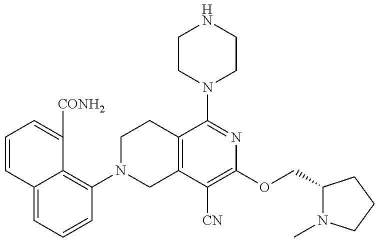 [SMILES: CN1CCC[C@H]1COc1nc(N2CCNCC2)c2c(c1C#N)CN(c1cccc3cccc(C(N)=O)c13)CC2]

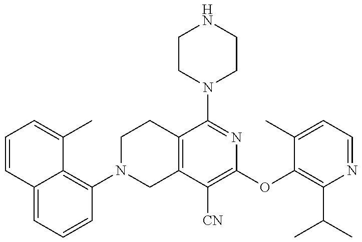 [SMILES: Cc1ccnc(C(C)C)c1Oc1nc(N2CCNCC2)c2c(c1C#N)CN(c1cccc3cccc(C)c13)CC2]

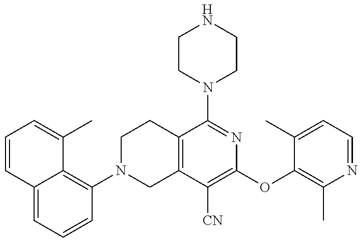 [SMILES: Cc1ccnc(C)c1Oc1nc(N2CCNCC2)c2c(c1C#N)CN(c1cccc3cccc(C)c13)CC2]

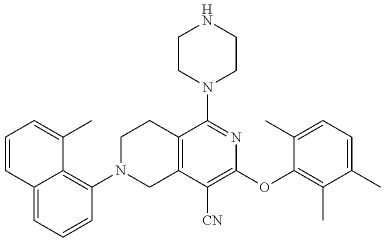 [SMILES: Cc1ccc(C)c(Oc2nc(N3CCNCC3)c3c(c2C#N)CN(c2cccc4cccc(C)c24)CC3)c1C]

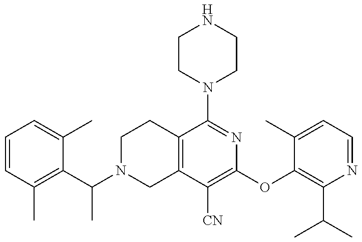 [SMILES: Cc1ccnc(C(C)C)c1Oc1nc(N2CCNCC2)c2c(c1C#N)CN(C(C)c1c(C)cccc1C)CC2]

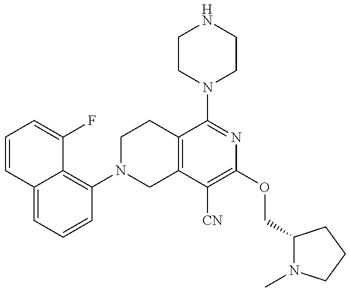 [SMILES: CN1CCC[C@H]1COc1nc(N2CCNCC2)c2c(c1C#N)CN(c1cccc3cccc(F)c13)CC2]

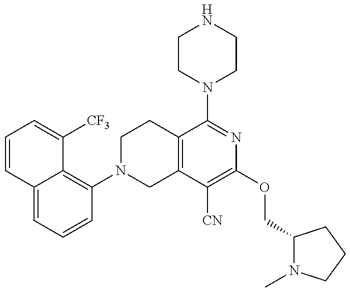 [SMILES: CN1CCC[C@H]1COc1nc(N2CCNCC2)c2c(c1C#N)CN(c1cccc3cccc(C(F)(F)F)c13)CC2]

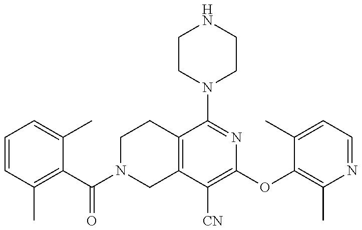 [SMILES: Cc1ccnc(C)c1Oc1nc(N2CCNCC2)c2c(c1C#N)CN(C(=O)c1c(C)cccc1C)CC2]

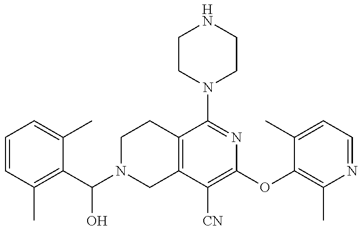 [SMILES: Cc1ccnc(C)c1Oc1nc(N2CCNCC2)c2c(c1C#N)CN(C(O)c1c(C)cccc1C)CC2]

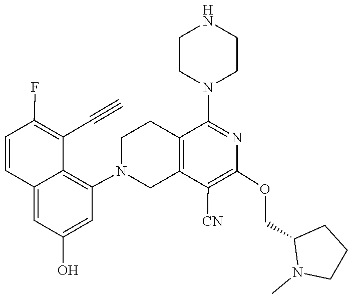 [SMILES: C#Cc1c(F)ccc2cc(O)cc(N3CCc4c(N5CCNCC5)nc(OC[C@@H]5CCCN5C)c(C#N)c4C3)c12]

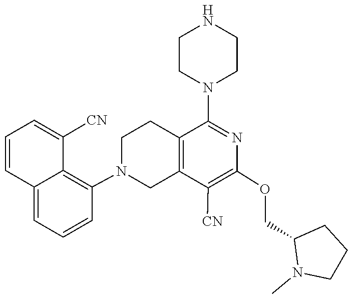 [SMILES: CN1CCC[C@H]1COc1nc(N2CCNCC2)c2c(c1C#N)CN(c1cccc3cccc(C#N)c13)CC2]

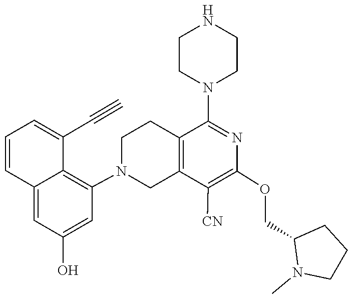 [SMILES: C#Cc1cccc2cc(O)cc(N3CCc4c(N5CCNCC5)nc(OC[C@@H]5CCCN5C)c(C#N)c4C3)c12]

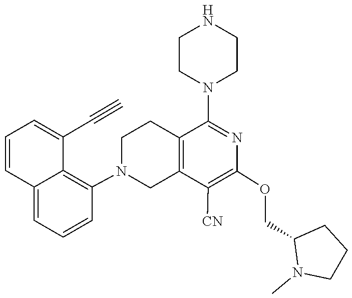 [SMILES: C#Cc1cccc2cccc(N3CCc4c(N5CCNCC5)nc(OC[C@@H]5CCCN5C)c(C#N)c4C3)c12]

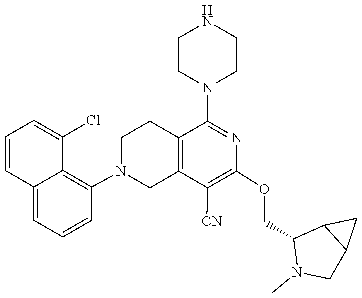 [SMILES: CN1CC2CC2[C@H]1COc1nc(N2CCNCC2)c2c(c1C#N)CN(c1cccc3cccc(Cl)c13)CC2]

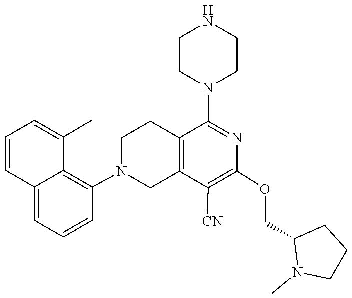 [SMILES: Cc1cccc2cccc(N3CCc4c(N5CCNCC5)nc(OC[C@@H]5CCCN5C)c(C#N)c4C3)c12]

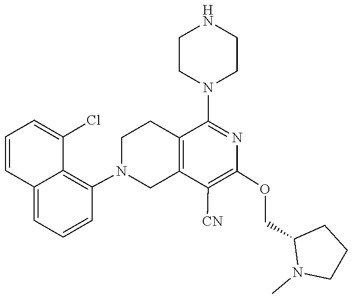 [SMILES: CN1CCC[C@H]1COc1nc(N2CCNCC2)c2c(c1C#N)CN(c1cccc3cccc(Cl)c13)CC2]

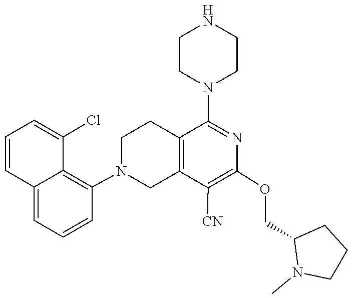 [SMILES: CN1CCC[C@H]1COc1nc(N2CCNCC2)c2c(c1C#N)CN(c1cccc3cccc(Cl)c13)CC2]

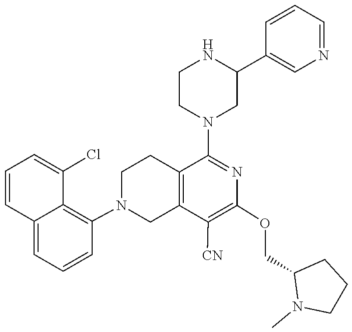 [SMILES: CN1CCC[C@H]1COc1nc(N2CCNC(c3cccnc3)C2)c2c(c1C#N)CN(c1cccc3cccc(Cl)c13)CC2]

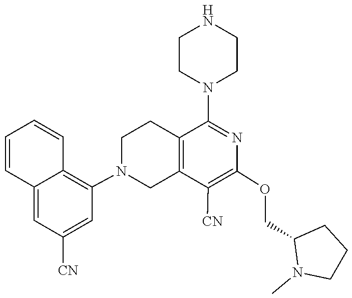 [SMILES: CN1CCC[C@H]1COc1nc(N2CCNCC2)c2c(c1C#N)CN(c1cc(C#N)cc3ccccc13)CC2]

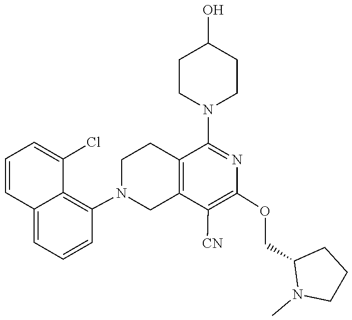 [SMILES: CN1CCC[C@H]1COc1nc(N2CCC(O)CC2)c2c(c1C#N)CN(c1cccc3cccc(Cl)c13)CC2]

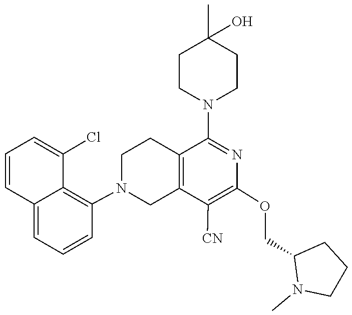 [SMILES: CN1CCC[C@H]1COc1nc(N2CCC(C)(O)CC2)c2c(c1C#N)CN(c1cccc3cccc(Cl)c13)CC2]

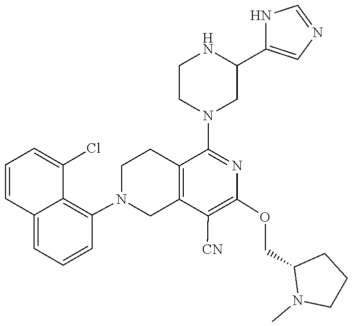 [SMILES: CN1CCC[C@H]1COc1nc(N2CCNC(c3cnc[nH]3)C2)c2c(c1C#N)CN(c1cccc3cccc(Cl)c13)CC2]

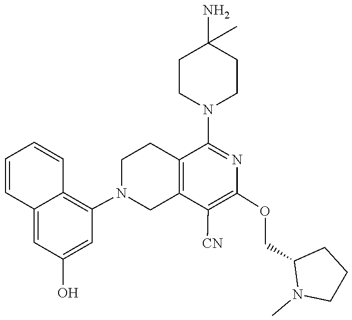 [SMILES: CN1CCC[C@H]1COc1nc(N2CCC(C)(N)CC2)c2c(c1C#N)CN(c1cc(O)cc3ccccc13)CC2]

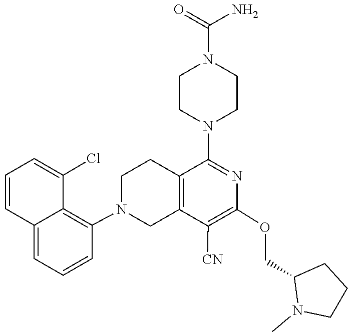 [SMILES: CN1CCC[C@H]1COc1nc(N2CCN(C(N)=O)CC2)c2c(c1C#N)CN(c1cccc3cccc(Cl)c13)CC2]

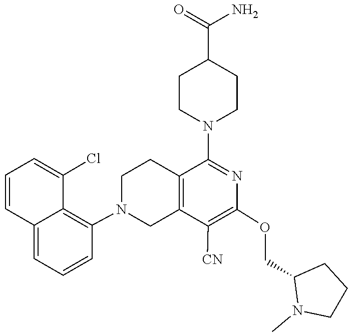 [SMILES: CN1CCC[C@H]1COc1nc(N2CCC(C(N)=O)CC2)c2c(c1C#N)CN(c1cccc3cccc(Cl)c13)CC2]

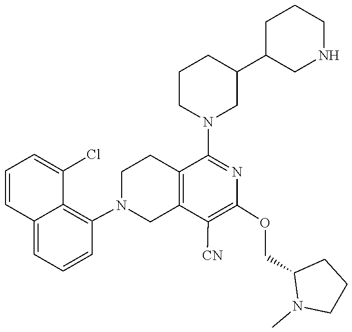 [SMILES: CN1CCC[C@H]1COc1nc(N2CCCC(C3CCCNC3)C2)c2c(c1C#N)CN(c1cccc3cccc(Cl)c13)CC2]

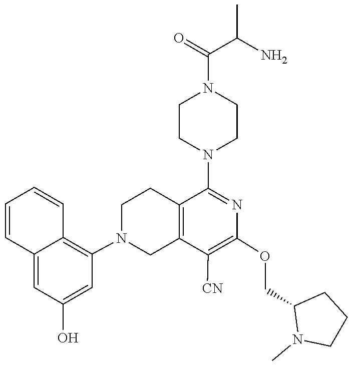 [SMILES: CC(N)C(=O)N1CCN(c2nc(OC[C@@H]3CCCN3C)c(C#N)c3c2CCN(c2cc(O)cc4ccccc24)C3)CC1]

Another embodiment of the invention is a composition comprising a compound of the invention, or a pharmaceutically acceptable salt thereof, and a pharmaceutically acceptable carrier.

Yet another embodiment of the invention is a method for treating cancer in a subject in need thereof, the method comprising administering to a subject in need thereof a therapeutically effective amount of a compound of the invention, or a pharmaceutically acceptable salt thereof, or a composition comprising a compound of the invention, or a pharmaceutically acceptable salt thereof.

Without being bound by a particular theory, provided herewith are the compounds that can modulate (e.g., inhibit) one or more members of the KRAS family, for example, one or more of KRAS mutants. More specifically, and without being bound by a particular theory, it is believed that the compounds described herein can bind to KRAS G12 D and function as covalent inhibitor of KRAS G12 D.

As such, in another embodiment, the invention is a method of treating a KRAS mediated disorder in a subject in need thereof, in particular KRAS G12 D mediated disorder comprising administering to the subject in need thereof a therapeutically effective amount of a compound of the invention, or a pharmaceutically acceptable salt thereof, or a pharmaceutical composition comprising a compound of the invention, or a pharmaceutically acceptable salt thereof.

Another embodiment of the invention is use of a compound of the invention for treating cancer or a RAS-mediated disorder, in particular KRAS G12 D mediated disorder in a subject.

Another embodiment of the invention is use of a compound of the invention for the manufacture of a medicament for treating cancer or a RAS mediated disorder, in particular KRAS G12 D mediated disorder in a subject.

Compounds of the present invention, and pharmaceutically acceptable salts and/or compositions thereof, are useful for treating a variety of cancers, such as solid cancer and, more specifically, solid cancers with KRASG12 mutation.

### DETAILED DESCRIPTION

A description of example embodiments of the invention follows.

### Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 7 5th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

Compounds of the present invention may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers or enantiomers, with all possible isomers and mixtures thereof, including optical isomers, being included in the present invention.

As used herein the following definitions shall apply unless otherwise indicated. Further many of the groups defined herein can be optionally substituted. The listing of substituents in the definition is exemplary and is not to be construed to limit the substituents defined elsewhere in the specification.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₃alkyl” refers to an alkyl group as defined above having up to 3 carbon atoms. The term “C₁₋₆alkyl” refers to an alkyl group as defined above having up to 6 carbon atoms. In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆alkenyl” refers to an alkenyl group as defined above having up to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “C₂₋₆ alkynyl” refers to an alkynyl group as defined above having up to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., —O-(substituted alkyl). For example “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., sprio (4,4) non-2-yl. The term “C₃₋₆ cycloalkyl” refers to a cycloalkyl group as defined above having up to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 up to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “G”, unless otherwise specified, refers to a basic group capable of forming a interaction with amino acid such as Aspartic acid or a chemical moiety which is capable of forming a interaction with amino acid such as Aspartic acid including a possibility of said interaction been a covalent bond formation including a group which is an electrophile or an electrophilic moiety capable of forming a covalent bond with amino acid such as Aspartic acid.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom.

The term “heterocyclyl”, unless otherwise specified, refers to a heterocylic ring radical as defined above. The heterocylcyl ring radical may be attached to the main structure at any heteroatom or carbon atom.

The term “heterocyclylalkyl”, unless otherwise specified, refers to a heterocylic ring radical as defined above directly bonded to an alkyl group. The heterocyclylalkyl radical may be attached to the main structure at any carbon atom in the alkyl group. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a heteroaryl ring radical as defined above directly bonded to an alkyl group. The heteroarylalkyl radical may be attached to the main structure at any carbon atom from alkyl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —SO₂NR^{x}R^{y}—, —ORₓ, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{x}, R^{y} and R^{z} may be joined to form a substituted or unsubstituted saturated or unsaturated 3-10 membered ring, which may optionally include heteroatoms which may be the same or different and are selected from O, NR^{x}(e.g., R^{x} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo.

The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The term “Amino acid” used here in constitute a group wherein the said group comprises of an amino moiety (NH₂) and an acid (—COOH) moiety with or without a characteristic stereochemistry and each of said amino moiety (NH₂) and an acid (—COOH) moiety may optionally be substituted such as for e.g resulting in formation of an amide or ester bond or are having a suitable protecting group or optionally substituted with one or more of Amino acid as defined herein. More preferably, the Amino acid is the one in which the amino and acid moiety are positioned on a same carbon or in other words the amino moiety is at the alfa (α) position to the carboxyl group with or without a characteristic stereochemistry; term Amino acid also include compound wherein the amino moiety is on the beta (0) position to the carboxyl group with or without a characteristic stereochemistry. for e.g the list of amino acids comprises of but not limited to Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine and Valine including both D and L isomers. In addition, other amino acids are modified amino acids and may be termed as non-protein amino acids or unnatural amino acids and may occur in nature or are synthetically made for e.g. chemical moieties having characteristics of amino acids can also be synthesized or commercially available. Beyond the known naturally occurring 20 amino acids, there are more than 300 other amino acids reported in literature either occurring naturally or are synthesized musing chemically or using bio catalysis are intended to include with the definition of term Amino Acid.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, tert-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

The term “tautomers” refers to compounds, which are characterized by relatively easy interconversion of isomeric forms in equilibrium. These isomers are intended to be covered by this invention. “Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a 3C- or ¹⁴C-enriched carbon are within the scope of this invention. Such compounds are useful, for example, as analytical tools, as probes in biological assays, or as therapeutic agents in accordance with the present invention. For example, in the case of variable R, the (C₁—C₄) alkyl or the —O—(C₁—C₄) alkyl can be suitably deuterated (e.g., —CD₃, —OCD₃).

The compounds of the present invention may also contain unnatural proportions of atomic isotopes at one or more of atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are encompassed within the scope of the present invention.

The term “stereoisomers” is a general term for all isomers of an individual molecule that differ only in the orientation of their atoms in space. It includes mirror image isomers (enantiomers), geometric (cis/trans) isomers and isomers of compounds with more than one chiral center that are not mirror images of one another (diastereomers).

A “leaving group or atom” (Lg or lg) is any group or atom that will, under the reaction conditions, cleave from the starting material, thus promoting reaction at a specified site. Suitable examples of such groups unless otherwise specified are halogen atoms and mesyloxy, p-nitrobenzensulphonyloxy and tosyloxy groups.

“Prodrug” is meant to indicate a compound that may be converted under physiological conditions or by solvolysis to a biologically active compound described herein (e.g., compound of formula (A), (A-I), (A-II), (A-IIIA), (A-IIIB) or (A-IIIC)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organ ism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, Prodrug design is discussed generally in Hardma, et al. (Eds.), Goodman and Gilman's The Pharmacological Basis of Therapeutics, 9th ed., pp. 11-16 (1996) all of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

In some embodiments, prodrugs include compounds of (A), (A-I), (A-II), (A-IIIA), (A-IIIB) or (A-IIIC) having a phosphate, phosphoalkoxy, ester or boronic ester substituent. Without being bound by theory, it is believed that such substituents are converted to a hydroxyl group under physiological conditions. Accordingly, embodiments include any of the compounds disclosed herein, wherein a hydroxyl group has been replaced with a phosphate, phosphoalkoxy, ester or boronic ester group, for example a phosphate or phosphoalkoxy group. For example, in some embodiments a hydroxyl group on the R¹ moiety is replaced with a phosphate, phosphoalkoxy, ester or boronic ester group, for example a phosphate or alkoxy phosphate group.

The term “ester” refers to a compound, which is formed by reaction between an acid and an alcohol with elimination of water. An ester can be represented by the general formula RCOOR′.

These prodrugs and esters are intended to be covered within the scope of this invention.

Additionally, the instant invention also includes the compounds which differ only in the presence of one or more isotopically enriched atoms for example replacement of hydrogen with deuterium or tritium, or the replacement of a carbon by ^{13c}- or ^{14C}-enriched carbon.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

The following abbreviations and terms have the indicated meanings throughout; Abbreviations used herein have their conventional meaning within the chemical and biological arts.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. In one embodiment, the amount of compound administered ranges from about 0.1 mg to 5 g, from about 1 mg to 2.0 g, from about 100 mg to 1.5 g, from about 200 mg to 1.5 g, from about 400 mg to 1.5 g, and from about 400 mg to 1.0 g.

As used herein, “treatment,” “treating,” or “ameliorating” refers to an approach for obtaining beneficial or desired results including but not limited to therapeutic benefit and/or a prophylactic benefit. By therapeutic benefit is meant eradication or amelioration of the underlying disorder being treated. In addition a therapeutic benefit is also achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. These terms are used interchangeably.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, an amount of a compound effective to treat a disorder, or a “therapeutically effective amount” refers to an amount of the compound which is effective, upon single or multiple dose administration to a subject or a cell, in curing, alleviating, relieving or improving one or more symptoms of a disorder.

As used herein, an amount of a compound effective to prevent a disorder, or a “prophylactically effective amount” of the compound refers to an amount effective, upon single- or multiple-dose administration to the subject, in preventing or delaying the onset or recurrence of a disorder or one or more symptoms of the disorder.

As used herein, the term “subject” or “patient” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc., and companion animals (dog, cat, horse, etc.).

The methods described herein can be useful in both human therapeutics and veterinary applications (e.g., dogs, cats, cows, sheep, pigs, horses, goats, chickens, turkeys, ducks, and geese).

In some embodiments, the patient is a mammal, and in some embodiments, the patient is human.

“Radiation therapy” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium- 153-EDTMP), or high-energy radiation, including without limitation x-rays, gamma rays, and neutrons.

The term “pharmaceutically acceptable carrier” or “pharmaceutically acceptable excipient” includes, but is not limited to, any and all, a non-toxic solvent, dispersant, excipient, adjuvant, fillers, salts, disintegrants, binders, lubricants, glidants, wetting agents, controlled release matrices, colorants/flavoring, carriers, buffers, stabilizers, solubilizers, or other material and combinations thereof which is mixed with the active ingredient in order to permit the formation of a pharmaceutical composition, i.e., a dosage form capable of being administered to a patient. One example of such a carrier is pharmaceutically acceptable oil typically used for parenteral administration. Pharmaceutically acceptable carriers are well known in the art.

It is understood that substituents and substitution patterns on the compounds of the invention can be selected by one of ordinary skill in the art to provide compounds that are chemically stable and that can be readily synthesized by techniques known in the art, as well as those methods set forth below. In general, the term “substituted,” whether preceded by the term “optionally” or not, means that one or more hydrogens of the designated moiety are replaced with a suitable substituent. Unless otherwise indicated, an “optionally substituted group” can have a suitable substituent at each substitutable position of the group and, when more than one position in any given structure may be substituted with more than one substituent selected from a specified group, the substituent can be either the same or different at every position. Alternatively, an “optionally substituted group” can be unsubstituted.

Combinations of substituents envisioned by this invention are preferably those that result in the formation of stable or chemically feasible compounds. If a substituent is itself substituted with more than one group, it is understood that these multiple groups can be on the same carbon atom or on different carbon atoms, as long as a stable structure results. The term “stable,” as used herein, refers to compounds that are not substantially altered when subjected to conditions to allow for their production, detection, and, in certain embodiments, their recovery, purification, and use for one or more of the purposes disclosed herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds of this invention include salts derived from suitable inorganic and organic acids and bases that are compatible with the treatment of patients.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In some embodiments, exemplary inorganic acids which form suitable salts include, but are not limited thereto, hydrochloric, hydrobromic, sulfuric and phosphoric acid and acid metal salts such as sodium monohydrogen orthophosphate and potassium hydrogen sulfate. Ilustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2 phenoxybenzoic, p-toluenesulfonic acid and other sulfonic acids such as methanesulfonic acid and 2-hydroxyethanesulfonic acid. Either the mono- or di-acid salts can be formed, or such salts can exist in either a hydrated, solvated or substantially anhydrous form. In general, the acid addition salts of these compounds are more soluble in water and various hydrophilic organic solvents, and generally demonstrate higher melting points in comparison to their free base forms.

In some embodiments, acid addition salts of the compounds of formula (A), (A-I), (A-II), (A-IIIA), (A-IIIB) or (A-IIIC) are most suitably formed from pharmaceutically acceptable acids, and include, for example, those formed with inorganic acids, e.g., hydrochloric, sulfuric or phosphoric acids and organic acids e.g. succinic, maleic, acetic or fumaric acid.

Other non-pharmaceutically acceptable salts, e.g., oxalates can be used, for example, in the isolation of compounds of formula (A), (A-I), (A-II), (A-IIIA), (A-IIIB) or (A-IIIC) for laboratory use, or for subsequent conversion to a pharmaceutically acceptable acid addition salt. Also included within the scope of the invention are base addition salts (such as sodium, potassium and ammonium salts), solvates and hydrates of compounds of the invention. The conversion of a given compound salt to a desired compound salt is achieved by applying standard techniques, well known to one skilled in the art.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository The term “cell proliferation” refers to a phenomenon by which the cell number has changed as a result of division. This term also encompasses cell growth by which the cell morphology has changed (e.g., increased in size) consistent with a proliferative signal

The term “selective inhibition” or “selectively inhibit” refers to a biologically active agent refers to the agent's ability to preferentially reduce the target signaling activity as compared to off-target signaling activity, via direct or indirect interaction with the target

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients thereof.

When introducing elements disclosed herein, the articles “a,” “an,” “the,” and “said” are intended to mean that there are one or more of the elements. The terms “comprising,” “having” and “including” are intended to be open-ended and mean that there may be additional elements other than the listed elements.

For example, provided herein are methods of treating various cancers in mammals (including humans and non-humans), comprising administering to a patient in need thereof a compound of the invention, or a pharmaceutically acceptable salt thereof. Such cancers include hematologic malignancies (leukemias, lymphomas, myelomas, myelodysplastic and myeloproliferative syndromes) and solid tumors (carcinomas such as oral, gall bladder, prostate, breast, lung, colon, pancreatic, renal, ovarian as well as soft tissue and osteosarcomas, and stromal tumors).

### Pharmaceutical Compositions

The invention provides a pharmaceutical composition comprising one or more compounds of the present invention. The pharmaceutical composition may include one or more additional active ingredients as described herein. The pharmaceutical composition may be administered for any of the disorders described herein.

The subject pharmaceutical compositions are typically formulated to provide a therapeutically effective amount of a compound of the present invention as the active ingredient. Where desired, the pharmaceutical compositions contain a compound of the present invention as the active ingredient and one or more pharmaceutically acceptable carriers or excipients, such as inert solid diluents and filers, diluents, including sterile aqueous solution and various organic solvents, permeation enhancers, solubilizers and adjuvants.

The pharmaceutical compositions can be administered alone or in combination with one or more other agents, which are also typically administered in the form of pharmaceutical compositions. Where desired, the subject compounds and other agent(s) may be mixed into a preparation or both components may be formulated into separate preparations to use them in combination separately or at the same time.

Methods include administration of a compound of the present invention by itself, or in combination as described herein, and in each case optionally including one or more suitable diluents, fillers, salts, disintegrants, binders, lubricants, glidants, wetting agents, controlled release matrices, colorants/flavouring, carriers, excipients, buffers, stabilizers, solubilizers, and combinations thereof.

Preparations of various pharmaceutical compositions are known in the art., e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

The compounds or pharmaceutical composition of the present invention can be administered by any route that enables delivery of the compounds to the site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds can also be administered intraadiposally or intrathecally.

The compositions can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

### Method of Treatment

In further aspects, the present invention provides a use of a compound of the invention, or a pharmaceutically acceptable salt thereof, for the manufacture of a medicament for the treatment of cancer. In some embodiments, the present invention provides a use of a compound of the invention in the manufacture of a medicament for the treatment of any of cancer and/or neoplastic disorders. In further aspects, the present invention provides a compound of the present invention for use in the treatment of cancer. In some embodiments, the present invention provides a compound of the present invention for use in the treatment of cancer and/or neoplastic disorders.

A compound or composition described herein can be used to treat a neoplastic disorder. A “neoplastic disorder” is a disease or disorder characterized by cells that have the capacity for autonomous growth or replication, e.g., an abnormal state or condition characterized by proliferative cell growth. Exemplary neoplastic disorders include but are not limited to: carcinoma, sarcoma, metastatic disorders, Solid tumor such as oral, gall bladder, prostate, breast, lung, colon, pancreatic, renal, ovarian as well as soft tissue and osteosarcomas, and stromal tumors for e.g., tumors arising from prostate, brain, bone, colon, pancreas, lung, breast, ovarian, and liver origin, hematopoietic neoplastic disorders, e.g., leukemias, lymphomas, myelomas, myelodysplastic, myeloproliferative syndromes and other malignant plasma cell disorders, and metastatic tumors. Prevalent cancers include but not limited to: breast, prostate, colon, lung, liver, and pancreatic cancers. Treatment with the compound can be in an amount effective to ameliorate at least one symptom of the neoplastic disorder, e.g., reduced cell proliferation, reduced tumor mass, etc.

The disclosed methods are useful in the prevention and treatment of cancer, including for example, solid tumors, soft tissue tumors, and metastases thereof, as well as in familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers including but not limited to tumors such as lung, prostate, breast, brain, skin, cervical carcinomas, testicular carcinomas, etc. More particularly, cancers that may be treated by the compositions and methods of the invention include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Further preferred, the invention provides for methods for inhibiting KRAS activity in a cell, comprising contacting the cell in which inhibition of KRAS activity is desired with an effective amount of a compound of formula (A), (A-I), (A-II), (A-IIIA), (A-IIIB) or (A-IIIC) pharmaceutically acceptable salts thereof or pharmaceutical compositions containing the compound or pharmaceutically acceptable salt thereof.

Further preferred, the invention provides for methods for inhibiting KRAS G12D activity in a cell, comprising contacting the cell in which inhibition of KRAS G12D activity is desired with an effective amount of a compound of formula (A), (A-I), (A-II), (A-IIIA), (A-IIIB) or (A-IIIC) pharmaceutically acceptable salts thereof or pharmaceutical compositions containing the compound or pharmaceutically acceptable salt thereof.

In one embodiment, a cell in which inhibition of KRAS G12D activity is desired is contacted with an effective amount of a compound of formula (A), (A-I), (A-II), (A-IIIA), (A-IIIB) or (A-IIIC), to negatively modulate the activity of KRAS G12D.

Further preferred, a therapeutically effective amount of pharmaceutically acceptable salt or pharmaceutical compositions containing a compound of formula (A), (A-I), (A-II), (A-IIIA), (A-IIIB) or (A-IIIC), may be used.

By negatively modulating the activity of KRAS G12D, the methods described herein are designed to inhibit undesired cellular proliferation resulting from enhanced KRAS G12D activity within the cell. The cells may be contacted in a single dose or multiple doses in accordance with a particular treatment regimen to effect the desired negative modulation of KRAS G12D.

Further preferred, methods of treating cancer in a patient in need thereof, comprising administering to said patient a therapeutically effective amount of a compound of formula (A), (A-I), (A-II), (A-IIIA), (A-IIIB) or (A-IIIC), pharmaceutically acceptable salts thereof or pharmaceutical compositions comprising the compound or pharmaceutically acceptable salts thereof are provided.

The compositions and methods provided herein may be used for the treatment of a KRAS associated cancer in a patient in need thereof, comprising administering to said patient a therapeutically effective amount of a compound of formula (A), (A-I), (A-II), (A-IIIA), (A-IIIB) or (A-IIIC), pharmaceutically acceptable salts thereof or pharmaceutical compositions comprising the compound or pharmaceutically acceptable salts thereof are provided. In one embodiment, the KRAS G12D-associated cancer is lung cancer.

The compositions and methods provided herein may be used for the treatment of a KRAS G12D-associated cancer in a patient in need thereof, comprising administering to said patient a therapeutically effective amount of a compound of formula (A), (A-I), (A-II), (A-IIIA), (A-IIIB) or (A-IIIC), pharmaceutically acceptable salts thereof or pharmaceutical compositions comprising the compound or pharmaceutically acceptable salts thereof are provided. In one embodiment, the KRAS G12D-associated cancer is lung cancer.

The compositions and methods provided herein may be used for the treatment of a wide variety of cancers including but not limited to tumors such as lung, prostate, breast, brain, skin, cervical carcinomas, testicular carcinomas, etc. More particularly, cancers that may be treated by the compositions and methods of the invention include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Further preferred, the cancer is non-small cell lung cancer, colorectal cancer or pancreatic cancer.

Further preferred, the cancer is lung cancer or colorectal cancer.

Further preferred, the cancer is pancreatic cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The invention further provides herein a compound of formula (A), (A-I), (A-II), (A-IIIA), (A-IIIB) or (A-IIIC), or a pharmaceutically acceptable salt or solvate thereof, or a pharmaceutical composition thereof as defined herein for use in therapy.

The invention further provides herein a compound of formula (A), (A-I), (A-II), (A-IIIA), (A-IIIB) or (A-IIIC), or a pharmaceutically acceptable salt or solvate thereof or a pharmaceutical composition thereof as defined herein for use in the treatment of cancer.

The invention further provides herein is a compound of formula (A), (A-I), (A-II), (A-IIIA), (A-IIIB) or (A-IIIC), or a pharmaceutically acceptable salt or solvate thereof for use in the inhibition of KRAS G12D.

The invention further provides herein is a compound of formula (A), (A-I), (A-II), (A-IIIA), (A-IIIB) or (A-IIIC), or a pharmaceutically acceptable salt or solvate thereof or a pharmaceutical composition thereof as defined herein, for use in the treatment of a KRAS G12D-associated disease or disorder.

The invention further provides herein is the use of a compound of formula (A), (A-I), (A-II), (A-IIIA), (A-IIIB) or (A-IIIC), or a pharmaceutically acceptable salt or solvate thereof, as defined herein in the manufacture of a medicament for the treatment of cancer.

The invention further provides herein is a use of a compound of formula (A), (A-I), (A-II), (A-IIIA), (A-IIIB) or (A-IIIC), or a pharmaceutically acceptable salt or solvate thereof, as defined herein in the manufacture of a medicament for the inhibition of activity of KRAS G12D.

The invention further provides herein is the use of a compound of formula (A), (A-I), (A-II), (A-IIIA), (A-IIIB) or (A-IIIC), or a pharmaceutically acceptable salt or solvate thereof, as defined herein, in the manufacture of a medicament for the treatment of a KRAS G12D-associated disease or disorder.

The invention further provides herein is a method for treating cancer in a patient in need thereof, the method comprising (a) determining that cancer is associated with a KRAS G12D mutation (e.g., a KRAS G12D-associated cancer) (e.g., as determined using a regulatory agency-approved, e.g., FDA-approved, assay or kit); and (b) administering to the patient a therapeutically effective amount of a compound of formula (A), (A-I), (A-II), (A-IIIA), (A-IIIB) or (A-IIIC), or a pharmaceutically acceptable salt thereof, or a pharmaceutical composition thereof.

Metastases of the aforementioned cancers can also be treated or prevented in accordance with the methods described herein. COMBINATION THERAPIES

In some embodiments, a compound described herein is administered together with an additional “second” therapeutic agent or treatment. The choice of second therapeutic agent may be made from any agent that is typically used in a monotherapy to treat the indicated disease or condition. As used herein, the term “administered together” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of any of the formulas described herein, an additional therapeutic agent, and a pharmaceutically acceptable carrier, adjuvant, or vehicle.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art. The additional agents may be administered separately, as part of a multiple dose regimen, from the compounds of this invention. Alternatively, those agents may be part of a single dosage form, mixed together with the compounds of this invention in a single composition.

The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The compounds, pharmaceutically acceptable salts thereof and pharmaceutical compositions comprising such compounds comprising the step of administering simultaneously or sequentially to a subject in need thereof at least one other anti-cancer agent, anti-inflammatory agent, immunosuppressive agent, steroid, non-steroidal anti-inflammatory agent, antihistamine, analgesic, or a mixture thereof.

### Process of Preparation of the Compound of the Invention

The instant compounds of the present invention may be prepared by the following general process. The process provided herein can similarly be applied to synthesize all possible variation of the compound of the invention, and in particular compounds of formulas (A) as provided herein above with all intended modification or without any modification. Unless otherwise indicated, the variables such as R, R^{a}, R¹, A₁, A₂, Cy¹, Cy² and E used here in various intermediates or compounds of formulas are to be constructed to be the variables defined herein above in relation to compound of the invention and in particular to the compound of formula (A).

Synthetic Scheme A: This schematic representation of scheme aims to provide a possible route for the preparation of a compound of formula (A)

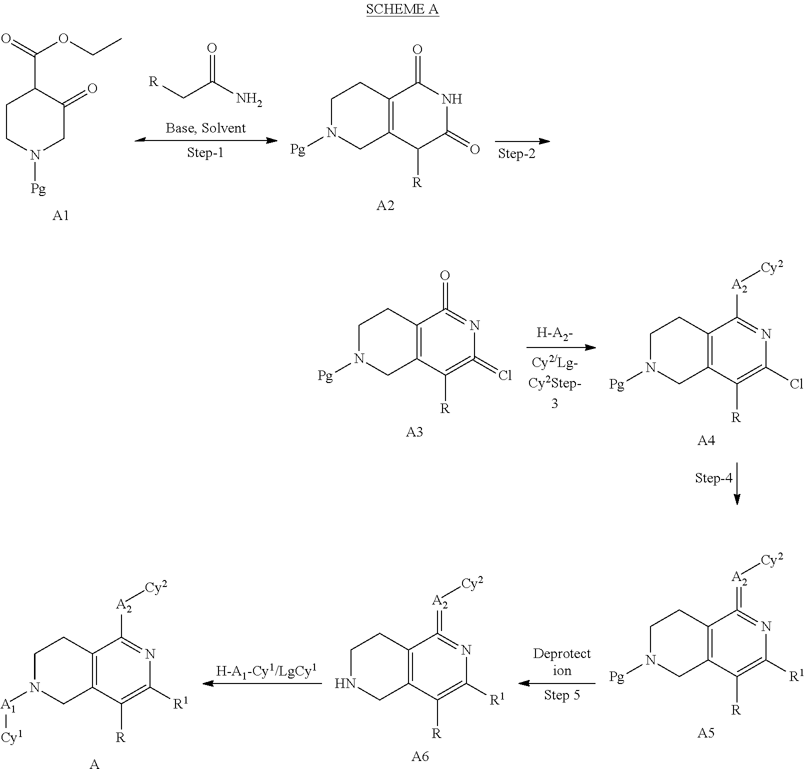

Intermediate of formula (A₁) where in Pg is a suitable protecting group when reacted with an intermediate of formula R—CH₂—CONH₂, using a suitable base or reagent to get an intermediate of formula (A₂). Intermediate of formula (A₂) can then be subjected to chlornation using POCl₃ to get an intermediate of formula (A₃) which on reacting with an intermediate f formula Lg-A₂—Cy² wherein Lg is a good leaving group using a suitable base to get an intermediate of formula (A₄). Intermediate of formula (A₄) is in turn can be coupled with an intermediate of formula Lg-R¹ wherein Lg is a good leaving group, using a suitable base to get an Intermediate of formula (A₅). Intermediate of formula (A₅). Intermediate (A₅) can then be de-protected followed by coupling with a group Lg-A₁—Cy¹ under suitable conditions such as Buchwald coupling or other suitable N-Alkylation or N-Arylation to provide a desired compound of formula (A).

### A. Scheme of Example 1 Provides for Illustrative Use of Scheme a as Below

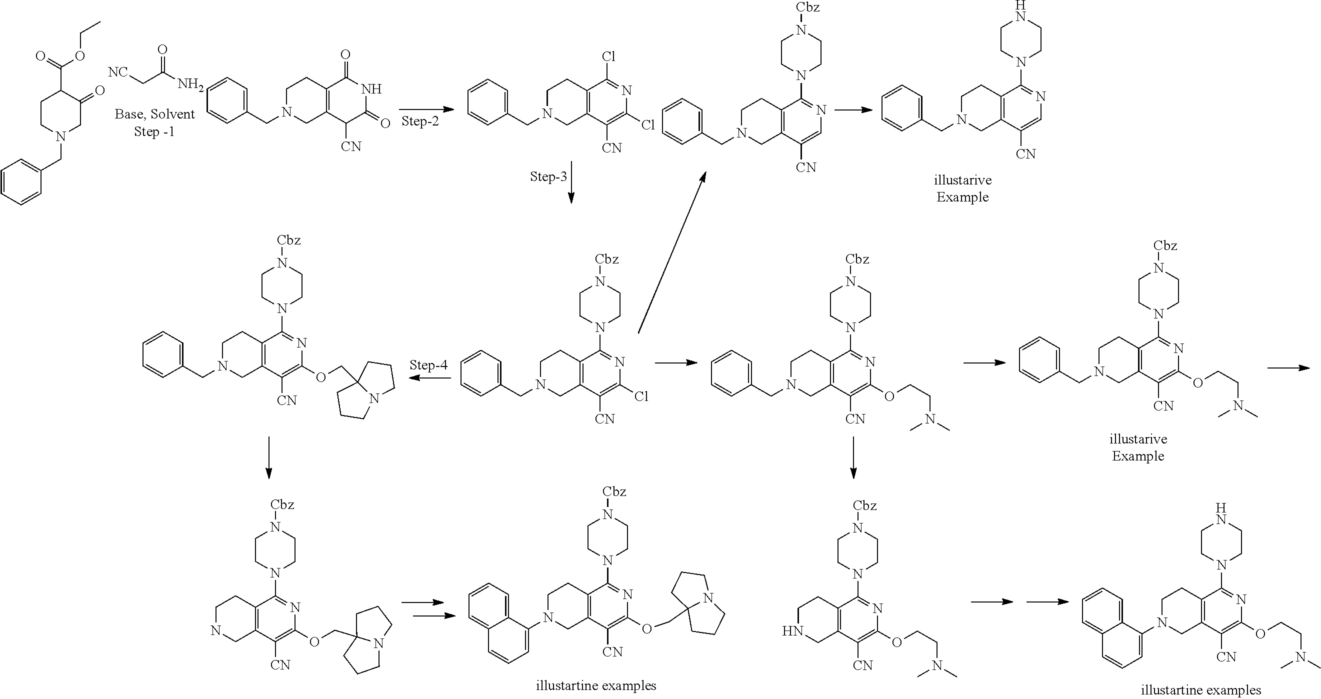

### B. Scheme Below Provides for Yet Another Illustrative Use of Scheme a to Prepare a Compound of Invention

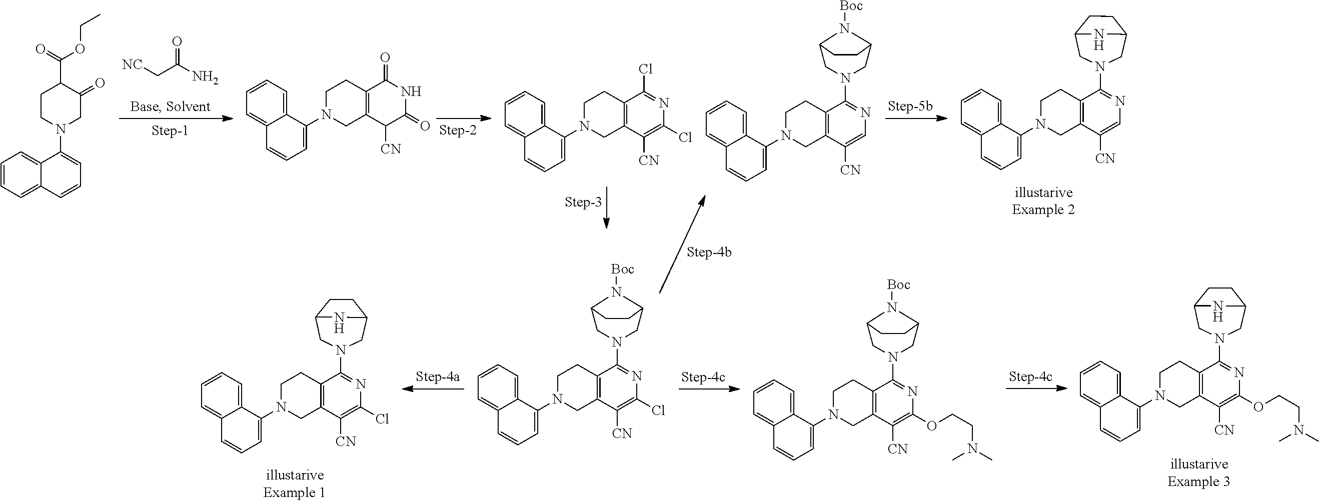

### C. Scheme Below Provides for Yet Another Illustrative Use of Scheme a to Prepare a Compound of Invention

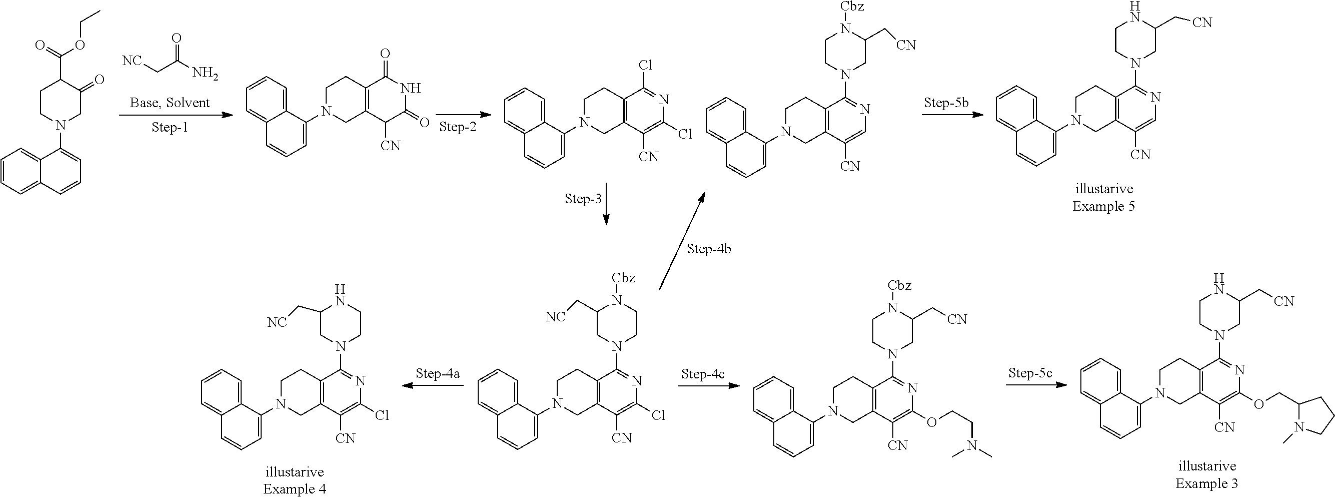

### Experimental Details

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Part-A: Preparation of Intermediates

Intermediate A: 6-benzyl-1,3-dioxo-1,2,3,4,5,6,7,8-octahydro-2,6-naphthyridine-4-carbonitrile: ethyl 1-benzyl-3-oxopiperidine-4-carboxylate (300 g, 1.14 mol) was added in the 2-necked flask containing methanol (2250 ml), to which cyanoacetamide (96 g, 1.14 mol) was added followed by addition of a potassium hydroxide solution in methanol (79 g, 1.43 mol of KOH in 1485 mL MeOH). The resulting mixture was stirred and heated to reflux for 16 hr. The reaction mixture was cooled to room temperature white solid which was filtered and washed with methanol (250 mL). The white solid obtained was dried under vacuum to get 430 g of KOH salt 6-benzyl-1,3-dioxo-1,2,3,4,5,6,7,8-octahydro-2,6-naphthyridine-4-carbonitrile. KOH salt was taken in 3000 ml water and heated upto 92° cand acidified with Conc HCl, then filtered, washed with 500 ml water, dried under oven overnight. Then extremely dried Intermediate 1 230 gm, salt free) of 6-benzyl-1,3-dioxo-1,2,3,4,5,6,7,8-octahydro-2,6-naphthyridine-4-carbonitrile.MS (m/z): 282.20 [M+H]+.

Intermediate B: 6-benzyl-1,3-dichloro-5,6,7,8-tetrahydro-2,6-naphthyridine-4-carbonitrile: Intermediate 1 (20 gm g, 0.071 mmol) tetramethylammonium chloride (9.3 g, 0.085 mol) and phosphorus oxychloride (7 mL, 0.71 mol) were taken in a sealed vessel and heated at 1250 C for 14 hrs. The reaction was cooled to RT, slowly open the vessel and concentrated under reduced pressure. The concentrated residue was poured into crushed ice and stirred vigorously with a glass rode. Then the saturated solution of NaOH (around 500 mL) was added slowly to the cold and diluted RM to turn it alkaline (pH=8-10). Solid gets precipitate, filtered off and dried overnight on oven to get Intermediate 2 (17 gm) as alight green solid. MS (m/z): 318.20 [M+H]⁺.

Intermediate-1: Synthesis of tert-butyl (1R,5S)-3-(6-benzyl-3-chloro-4-cyano-5,6,7,8-tetrahydro-2,6-naphthyridin-1-yl)-3,8-diazabicyclo[3.2.1]octane-8-carboxylate: To a solution of 6-benzyl-1,3-dichloro-5,6,7,8-tetrahydro-2,6-naphthyridine-4-carbonitrile in THF. tert-butyl (1R,5S)-3,8-diazabicyclo[3.2.1]octane-8-carboxylate and DIPEA was added. Reaction mixture was heated at 60° C. for 12 h. Reaction was poured into water and extracted with Ethyl acetate(3X), mix organic layer dried under Na₂SO₄ and evaporated under vacuum, crude was purified by column chromatography and eluted at (1:4) Ethyl aetate: n-hexane to get tert-butyl (1R,5S)-3-(6-benzyl-3-chloro-4-cyano-5,6,7,8-tetrahydro-2,6-naphthyridin-1-yl)-3,8-diazabicyclo[3.2.1]octane-8-carboxylate. Mass: 495.1 [m+1].

Intermediate-1A: tert-butyl (1R,5S)-8-(6-benzyl-3-chloro-4-cyano-5,6,7,8-tetrahydro-2,6-naphthyridin-1-yl)-3,8-diazabicyclo[3.2.1]octane-3-carboxylate: To a solution of 6-benzyl-1,3-dichloro-5,6,7,8-tetrahydro-2,6-naphthyridine-4-carbonitrile in THF. tert-butyl (1R,5S)-3,8-diazabicyclo[3.2.1]octane-3-carboxylate and DIPEA was added. Reaction mixture was heated at 60° C. for 12 h. Reaction was poured into water and extracted with Ethyl acetate(3X), mix organic layer dried under Na₂SO₄ and evaporated under vacuum, crude was purified by column chromatography and eluted at (1:4) Ethyl aetate: n-hexane to get tert-butyl (1R,5S)-8-(6-benzyl-3-chloro-4-cyano-5,6,7,8-tetrahydro-2,6-naphthyridin-1-yl)-3,8-diazabicyclo[3.2.1]octane-3-carboxylate.Mass: 495.0 [m+1].

Intermediate-2: tert-butyl (1R,5S)-3-(6-benzyl-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate: To a solution of (S)-(1-methylpyrrolidin-2-yl)methanol in THE was cooled at 0° C. NaH was added portion wise and tert-butyl (1R,5S)-3-(6-benzyl-3-chloro-4-cyano-5,6,7,8-tetrahydro-2,6-naphthyridin-1-yl)-3,8-diazabicyclo[3.2.1]octane-8-carboxylate was dissolved in THE and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl (1R,5S)-3-(6-benzyl-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate. Mass: 573.2 [m+1].

Intermediate-2A: tert-butyl (1R,5S)-8-(6-benzyl-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution of (S)-(1-methylpyrrolidin-2-yl)methanol in THE was cooled at 0° C. NaH was added portion wise and tert-butyl (1R,5S)-8-(6-benzyl-3-chloro-4-cyano-5,6,7,8-tetrahydro-2,6-naphthyridin-1-yl)-3,8-diazabicyclo[3.2.1]octane-3-carboxylate was dissolved in THE and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl (1R,5S)-8-(6-benzyl-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate. Mass: 573.4 [m+1]. To a solution of ((2R,7aS)-2-fluorotetrahydro-1H-pyrrolizin-7a(5H)-yl)methanol in THE was cooled at 0° C. NaH was added portion wise and tert-butyl (1R,5S)-3-(6-benzyl-3-chloro-4-cyano-5,6,7,8-tetrahydro-2,6-naphthyridin-1-yl

Intermediate-3: tert-butyl (1R,5S)-3-(6-benzyl-4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate: To a solution of ((2R,7aS)-2-fluorotetrahydro-1H-pyrrolizin-7a(5H)-yl)methanol in THF was cooled at 0° C. NaH was added portion wise and tert-butyl (1R,5S)-3-(6-benzyl-3-chloro-4-cyano-5,6,7,8-tetrahydro-2,6-naphthyridin-1-yl)-3,8-diazabicyclo[3.2.1]octane-8-carboxylate was dissolved in THE and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl (1R,5S)-3-(6-benzyl-4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate. Mass: 617.3 [m+1].

Intermediate-3A: tert-butyl (1R,5S)-8-(6-benzyl-4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution of (tetrahydro-1H-pyrrolizin-7a(5H)-yl)methanol in THE was cooled at 0° C. NaH was added portion wise and tert-butyl (1R,5S)-8-(6-benzyl-3-chloro-4-cyano-5,6,7,8-tetrahydro-2,6-naphthyridin-1-yl)-3,8-diazabicyclo[3.2.1]octane-3-carboxylate was dissolved in THE and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl (1R,5S)-8-(6-benzyl-4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate. Mass: 599.3 [m+1].

Intermediate-4: tert-butyl (1R,5S)-3-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-8-carboxylate: To a solution of cyclopropane-1,1-diyldimethanol in THE was cooled at 0° C. NaH was added portion wise and tert-butyl (1R,5S)-3-(6-benzyl-3-chloro-4-cyano-5,6,7,8-tetrahydro-2,6-naphthyridin-1-yl)-3,8-diazabicyclo[3.2.1]octane-8-carboxylate was dissolved in THF and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at 30% ethyl acetate: Pet ether to get tert-butyl (1R,5S)-3-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-8-carboxylate. Mass: 560.2 [m+1].

Intermediate-4A: tert-butyl (1R,5S)-8-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-3-carboxylate: To a solution of cyclopropane-1,1-diyldimethano in THE was cooled at 0° C. NaH was added portion wise and tert-butyl (1R,5S)-3-(6-benzyl-3-chloro-4-cyano-5,6,7,8-tetrahydro-2,6-naphthyridin-1-yl)-3,8-diazabicyclo[3.2.1]octane-3-carboxylate was dissolved in THF and added dropwise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at 30% ethyl acetate: Pet ether to get tert-butyl (1R,5S)-8-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-3-carboxylate. Mass: 560.3[m+1].

Intermediate-5: tert-butyl (1R,5S)-3-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-8-carboxylate: To a solution of tert-butyl (1R,5S)-3-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-8-carboxylate in DCM was cooled at 0° C. Triethylamine and Methanesulfonyl chloride was added dropwise wise. Reaction mixture was stirred at RT for 2 h. Reaction was poured into water and extracted with Ethyl acetate(3×), mix organic layer dried under Na₂SO₄ and evaporated under vacuum, get tert-butyl (1R,5S)-3-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-8-carboxylate. Mass: 638.5 [m+1].

Intermediate-5A: tert-butyl (1R,5S)-8-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-3-carboxylate: To a solution of tert-butyl (1R,5S)-8-(6-benzyl-4-cyano-3-((1-(hydroxymethyl)cyclopropyl)methoxy)-5,6,7,8-tetrahydro-2,6-naphthyridin-1-yl)-3,8-diazabicyclo[3.2.1]octane-3-carboxylate in DCM was cooled at 0° C. Triethylamine and Methanesulfonyl chloride was added dropwise wise. Reaction mixture was stirred at RT for 2 h. Reaction was poured into water and extracted with Ethyl acetate(3×), mix organic layer dried under Na₂SO₄ and evaporated under vacuum, get tert-butyl (1R,5S)-8-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-3-carboxylate. Mass: 638.6 [m+1].

Intermediate-6: tert-butyl (1R,5S)-3-(6-benzyl-4-cyano-3-((1-(morpholinomethyl)cyclopropyl)methoxy)-5,6,7,8-tetrahydro-2,6-naphthyridin-1-yl)-3,8-diazabicyclo[3.2.1]octane-8-carboxylate: To a solution of tert-butyl (1R,5S)-3-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-8-carboxylate in THE was cooled at 0° C. DIPEA and Morpholine was added dropwise wise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at 1% MeOH:DCM get tert-butyl (1R,5S)-3-(6-benzyl-4-cyano-3-((1-(morpholinomethyl)cyclopropyl)methoxy)-5,6,7,8-tetrahydro-2,6-naphthyridin-1-yl)-3,8-diazabicyclo[3.2.1]octane-8-carboxylate. Mass: 629.6 [m+1].

Intermediate-6A: tert-butyl (1R,5S)-8-(6-benzyl-4-cyano-3-((1-(morpholinomethyl)cyclopropyl)methoxy)-5,6,7,8-tetrahydro-2,6-naphthyridin-1-yl)-3,8-diazabicyclo[3.2.1]octane-3-carboxylate: To a solution of tert-butyl (1R,5S)-8-(6-benzyl-4-cyano-3-((1-(((methylsulfonyl)oxy)methyl)cyclopropyl)methoxy)-5,6,7,8-tetrahydro-2,6-naphthyridin-1-yl)-3,8-diazabicyclo[3.2.1]octane-3-carboxylate in THE was cooled at 0° C. DIPEA and Morpholine was added dropwise wise. Reaction mixture was heated at 60° C. for 12 h. Reaction was poured into water and extracted with Ethyl acetate(3×), mix organic layer dried under Na₂SO₄ and evaporated under vacuum, crude was purified by column chromatography and eluted at 1% MeOH:DCM get tert-butyl (1R,5S)-8-(6-benzyl-4-cyano-3-((1-(morpholinomethyl)cyclopropyl)methoxy)-5,6,7,8-tetrahydro-2,6-naphthyridin-1-yl)-3,8-diazabicyclo[3.2.1]octane-3-carboxylate. Mass: 629.4 [m+1].

Intermediate 6B: tert-butyl 4-(4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate: Intermediate 5a was prepared using similar methodoly as descrived herein above for Intermediate 5 using Intermediate 2 with some modification and using specific chemicals and reagents. This was used in next step without analytical data.

Intermediate-7: tert-butyl (1R,5S)-3-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate: To a solution of tert-butyl (1R,5S)-3-(6-benzyl-4-cyano-3-(((S)-1-methylpyrrolidin-2-yl)methoxy)-5,6,7,8-tetrahydro-2,6-naphthyridin-1-yl)-3, 8-diazabicyclo[3.2.1]octane-8-carboxylate in Methanol. Nitrogen gas was purged for 10 min. 10% Pd/c was added and H₂ bladder was put over reaction flask. Reaction mixture was stirred at RT for 12 h. Reaction was filtered through celite pad, filtrate evaporated under vacuum to get tert-butyl (1R,5S)-3-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate. Mass: 483.1 [m+1].

Intermediate-7A: tert-butyl (1R,5S)-8-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution of tert-butyl (1R,5S)-8-(6-benzyl-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate in Methanol. Nitrogen gas was purged for 10 min. 10% Pd/c was added and H₂ bladder was put over reaction flask. Reaction mixture was stirred at RT for 12 h. Reaction was filtered through celite pad, filtrate evaporated under vacuum to get tert-butyl (1R,5S)-8-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate. Mass: 483.1[m+1].

Intermediate-8: Synthesis of tert-butyl (1R,5S)-3-(4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate: To a solution of tert-butyl (1R,5S)-3-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate in Methanol. Nitrogen gas was purged for 10 min. 10% Pd/c was added and H₂ bladder was put over reaction flask. Reaction mixture was stirred at RT for 12 h. Reaction was filtered through celite pad, filtrate evaporated under vacuum to get tert-butyl (1R,5S)-3-(4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate. Mass: 527.3 [m+1].

Intermediate-8A: tert-butyl (1R,5S)-8-(4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution of tert-butyl (1R,5S)-8-(6-benzyl-4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in Methanol. Nitrogen gas was purged for 10 min. 10% Pd/c was added and H₂ bladder was put over reaction flask. Reaction mixture was stirred at RT for 12 h. Reaction was filtered through celite pad, filtrate evaporated under vacuum to get tert-butyl (1R,5S)-8-(4-cyano-3-((tetrahydro-1H-pyrrolizin-7a(5H)-yl)methoxy)-5,6,7,8-tetrahydro-2,6-naphthyridin-1-yl)-3, 8-diazabicyclo[3.2.1]octane-3-carboxylate. Mass: 509.4 [m+1].

Intermediate-9: tert-butyl (1R,5S)-3-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate: To a solution tert-butyl (1R,5S)-3-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate and 1-bromo-8-chloronaphthalene in Toluene. Cs2CO3 was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-3-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate. Mass: 644.1 [M−1].

Intermediate-10: tert-butyl (1R,5S)-3-(6-(8-chloronaphthalen-1-yl)-4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate: To a solution tert-butyl (1R,5S)-3-(4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate and 1-bromo-8-chloronaphthalene in Toluene. Cs2CO3 was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-3-(6-(8-chloronaphthalen-1-yl)-4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate. Mass: 688.2 [M−1].

Intermediate-11: tert-butyl (1R,5S)-3-(4-cyano-3-(((2R,7aS)-2-fluorotetrahydro-1H-pyrrolizin-7a(5H)-yl)methoxy)-6-(3-hydroxynaphthalen-1-yl)-5,6,7,8-tetrahydro-2,6-naphthyridin-1-yl)-3,8-diazabicyclo[3.2.1]octane-8-carboxylate: To a solution tert-butyl (1R,5S)-3-(4-cyano-3-(((2R,7aS)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate and 4-bromonaphthalen-2-ol in Toluene. Cs2CO3 was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-3-(4-cyano-3-(((2R,7aS)-2-fluorotetrahydro-1H-pyrrolizin-7a(5H)-yl)methoxy)-6-(3-hydroxynaphthalen-1-yl)-5,6,7,8-tetrahydro-2,6-naphthyridin-1-yl)-3,8-diazabicyclo[3.2.1]octane-8-carboxylate. Mass: 669.5 [M−1].

Intermediate-11A: tert-butyl (1R,5S)-8-(4-cyano-6-(3-hydroxynaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate and 4-bromonaphthalen-2-ol in Toluene. Cs2CO3 was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(4-cyano-6-(3-hydroxynaphthalen-1-yl)-3-((tetrahydro-1H-pyrrolizin-7a(5H)-yl)methoxy)-5, 6,7,8-tetrahydro-2,6-naphthyridin-1-yl)-3,8-diazabicyclo[3.2.1]octane-3-carboxylate. Mass: 651.3 [M−1].

Intermediate-12A: tert-butyl (1R,5S)-8-(4-cyano-3-((tetrahydro-1H-pyrrolizin-7a(5H)-yl)methoxy)-6-(8-((triisopropylsilyl)ethynyl)naphthalen-1-yl)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate and ((8-bromonaphthalen-1-yl)ethynyl)triisopropylsilane in Toluene. Cs2CO3 was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(4-cyano-3-((tetrahydro-1H-pyrrolizin-7a(5H)-yl)methoxy)-6-(8-((triisopropylsilyl)ethynyl)naphthalen-1-yl)-5,6,7,8-tetrahydro-2,6-naphthyridin-1-yl)-3,8-diazabicyclo[3.2.1]octane-3-carboxylate. Mass: 816.1 [M−1].

Intermediate-13A: tert-butyl (1R,5S)-8-(4-cyano-6-(3-hydroxy-8-((triisopropylsilyl)ethynyl)naphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate and 4-bromo-5-((triisopropylsilyl)ethynyl)naphthalen-2-ol in Toluene. Cs₂CO₃ was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(4-cyano-6-(3-hydroxy-8-((triisopropylsilyl)ethynyl)naphthalen-1-yl)-3-((tetrahydro-1H-pyrrolizin-7a(5H)-yl)methoxy)-5,6,7,8-tetrahydro-2,6-naphthyridin-1-yl)-3, 8-diazabicyclo[3.2.1]octane-3-carboxylate. Mass: 832.1 [M−1].

Intermediate-14A: tert-butyl (1R,5S)-8-(4-cyano-3-(((S)-1-methylpyrrolidin-2-yl)methoxy)-6-(8-((triisopropylsilyl)ethynyl)naphthalen-1-yl)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate and ((8-bromonaphthalen-1-yl)ethynyl)triisopropylsilane in Toluene. Cs₂CO₃ was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(4-cyano-3-(((S)-1-methylpyrrolidin-2-yl)methoxy)-6-(8-((triisopropylsilyl)ethynyl)naphthalen-1-yl)-5,6,7,8-tetrahydro-2,6-naphthyridin-1-yl)-3,8-diazabicyclo[3.2.1]octane-3-carboxylate. Mass: 790.1 [M−1].

Intermediate-15A: tert-butyl (1R,5S)-8-(6-(8-bromonaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate and 1,8-dibromonaphthalene in Toluene. Cs₂CO₃ was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(6-(8-bromonaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate. Mass: 688.3 [M−1].

Intermediate-16A: tert-butyl (1R,5S)-8-(6-(8-bromo-6-hydroxynaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate and 4,5-dibromonaphthalen-2-ol in Toluene. Cs₂CO₃ was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(6-(8-bromo-6-hydroxynaphthalen-1-yl)-4-cyano-3-(((S)-1-methylpyrrolidin-2-yl)methoxy)-5,6,7,8-tetrahydro-2,6-naphthyridin-1-yl)-3, 8-diazabicyclo[3.2.1]octane-3-carboxylate. Mass: 704.2 [M−1].

Intermediate-17A: tert-butyl (1R,5S)-8-(4-cyano-6-(8-ethynylnaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-((tetrahydro-1H-pyrrolizin-7a(5H)-yl)methoxy)-6-(8-((triisopropylsilyl)ethynyl)naphthalen-1-yl)-5,6,7,8-tetrahydro-2,6-naphthyridin-1-yl)-3, 8-diazabicyclo[3.2.1]octane-3-carboxylate in DMF. To it CsF was added, reaction mixture was stirred at RT for 16 h. Reaction mixture diluted with water and extracted with (3×50 ml) EtOAc, combined organic layer washed with brine and dried over Na₂SO₄, evaporated under vacuum. Purification done by column chromatography, eluted at 3% DCM-Methanol to get tert-butyl (1R,5S)-8-(4-cyano-6-(8-ethynylnaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate. Mass: 659.4 [M−1].

Intermediate-18A: tert-butyl (1R,5S)-8-(4-cyano-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-6-(3-hydroxy-8-((triisopropylsilyl)ethynyl)naphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DMF. To it CsF was added, reaction mixture was stirred at RT for 16 h. Reaction mixture diluted with water and extracted with (3×50 ml) EtOAc, combined organic layer washed with brine and dried over Na₂SO₄, evaporated under vacuum. Purification done by column chromatography, eluted at 3% DCM-Methanol to get tert-butyl (1R,5S)-8-(4-cyano-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridin-1-yl)-3, 8-diazabicyclo[3.2.1]octane-3-carboxylate. Mass: 675.5 [M−1].

Intermediate-19A: tert-butyl (1R,5S)-8-(4-cyano-6-(8-ethynylnaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-(((S)-1-methylpyrrolidin-2-yl)methoxy)-6-(8-((triisopropylsilyl)ethynyl)naphthalen-1-yl)-5,6,7,8-tetrahydro-2,6-naphthyridin-1-yl)-3, 8-diazabicyclo[3.2.1]octane-3-carboxylate in DMF. To it CsF was added, reaction mixture was stirred at RT for 16 h. Reaction mixture diluted with water and extracted with (3×50 ml) EtOAc, combined organic layer washed with brine and dried over Na₂SO₄, evaporated under vacuum. Purification done by column chromatography, eluted at 3% DCM-Methanol to get tert-butyl (1R,5S)-8-(4-cyano-3-(((S)-1-methylpyrrolidin-2-yl)methoxy)-6-(8-((triisopropylsilyl)ethynyl)naphthalen-1-yl)-5,6,7,8-tetrahydro-2,6-naphthyridin-1-yl)-3,8-diazabicyclo[3.2.1]octane-3-carboxylate. Mass: 633.2 [M−1].

Intermediates 20A: tert-butyl 3-(6-(3-(benzyloxy)naphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution of tert-butyl 3-(4-cyano-3-(((S)-1-methylpyrrolidin-2-yl)methoxy)-5,6,7,8-tetrahydro-2,6-naphthyridin-1-yl)-3, 8-diazabicyclo[3.2.1]octane-8-carboxylate (0.5 g, 0.0010 mmol) in toluene (10 ml) purged under nitrogen gas for 20 min, was added 3-(benzyloxy)-1-bromonaphthalene (0.48 g, 0.0015 mmol), Cesium carbonate (1.01 gm, 0.0030 mmol) and Xantphos (0.12 gm, 0.0020 mmol) over a period of 10 min and stirred under Nitrogen atmosphere. Pd2(dba)3 (0.094 gm, 0.0001 mmol) was then added to the reaction mixture and heated at 120° C. for 12 hr. Reaction mixture was quenched with water and extracted with ethyl acetate, the extract was evaporated under reduced vacuum to get crude residue which was purified by column chromatography using MeOH: DCM (5%) to get desired (0.22 gm). MS (m/z): 714.6 [M+H]f.

Intermediate 21: tert-butyl 4-{6-benzyl-4-cyano-3-[(1-methylpyrrolidin-2-yl)methoxy]-5,6,7,8-tetrahydro-2,6-naphthyridin-1-yl}-3-methylpiperazine-1-carboxylate: To the solution of {[(2S)-1-methylpyrrolidin-2-yl]methanol} (0.1 g, 0.311 mmol) in Tetrahydrofuran was added Sodium hydride (0.622 mmol) in small portions while N2 gas was purged. To this solution of {tert-butyl 4-(6-benzyl-3-chloro-4-cyano-5,6,7,8-tetrahydro-2,6-naphthyridin-1-yl)-3-methylpiperazine-1-carboxylate} (0.207 mmol) in Tetrahydrofuran was added dropwise and reaction mixture was heated at 60° C. overnight Reaction mixture was monitored by TLC.. Reaction mixture was cooled to room temperature and was poured into water and extracted with Ethyl acetate (3*25 ml). Combined Organic layer were dried overNa2SO4 and concentrated under vacuum to give crude {tert-butyl 4-{6-benzyl-4-cyano-3-[(1-methylpyrrolidin-2-yl)methoxy]-5,6,7,8-tetrahydro-2,6-naphthyridin-1-yl}-3-methylpiperazine-1-carboxylate}. which was purified by coloumn chromatography to get 0.02 g of titled compound. MS (m/z): 562.2 [M+H]⁺.

TABLE 3

| | |
|---|---|
| 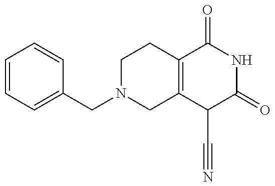 [SMILES: N#CC1C(=O)NC(=O)C2=C1CN(Cc1ccccc1)CC2] | Int A |
| 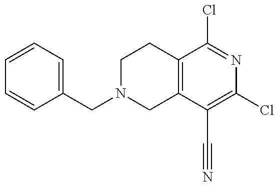 [SMILES: N#Cc1c(Cl)nc(Cl)c2c1CN(Cc1ccccc1)CC2] | Int B |
| 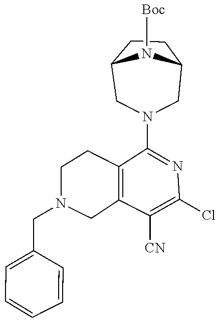 [SMILES: CC(C)(C)OC(=O)N1[C@@H]2CCC[C@H]1CN(c1nc(Cl)c(C#N)c3c1CCN(Cc1ccccc1)C3)C2] | Int-1 |
| 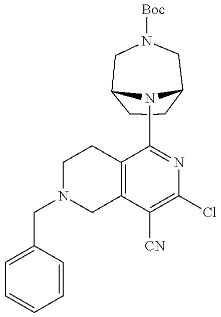 [SMILES: CC(C)(C)OC(=O)N1C[C@H]2CCC[C@@H](C1)N2c1nc(Cl)c(C#N)c2c1CCN(Cc1ccccc1)C2] | Int-1A |
| 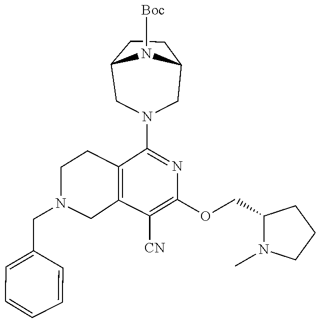 [SMILES: CN1CCC[C@H]1COc1nc(N2C[C@H]3CCC[C@@H](C2)N3C(=O)OC(C)(C)C)c2c(c1C#N)CN(Cc1ccccc1)CC2] | Int-2 |
| 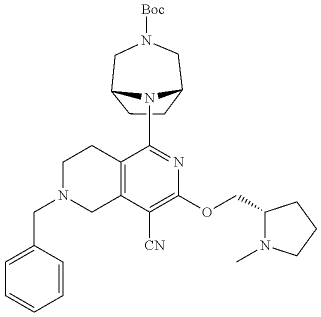 [SMILES: CN1CCC[C@H]1COc1nc(N2[C@@H]3CCC[C@H]2CN(C(=O)OC(C)(C)C)C3)c2c(c1C#N)CN(Cc1ccccc1)CC2] | Int-2A |
| 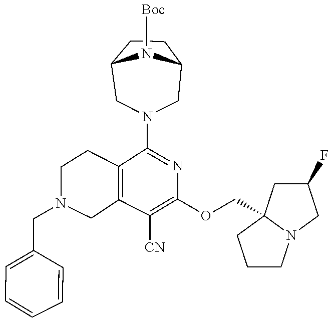 [SMILES: CC(C)(C)OC(=O)N1[C@@H]2CCC[C@H]1CN(c1nc(OC[C@@]34CCCN3C[C@H](F)C4)c(C#N)c3c1CCN(Cc1ccccc1)C3)C2] | Int-3 |
| 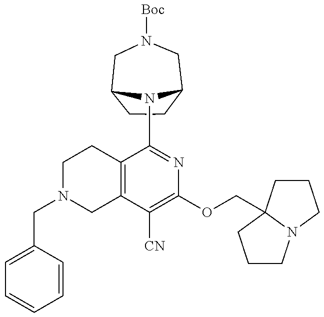 [SMILES: CC(C)(C)OC(=O)N1C[C@H]2CCC[C@@H](C1)N2c1nc(OCC23CCCN2CCC3)c(C#N)c2c1CCN(Cc1ccccc1)C2] | Int-3A |
| 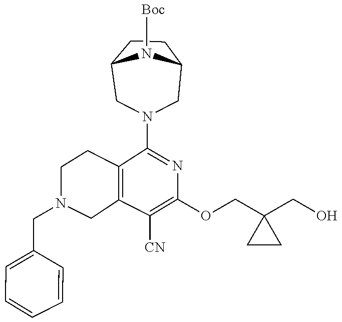 [SMILES: CC(C)(C)OC(=O)N1[C@@H]2CCC[C@H]1CN(c1nc(OCC3(CO)CC3)c(C#N)c3c1CCN(Cc1ccccc1)C3)C2] | Int-4 |
| 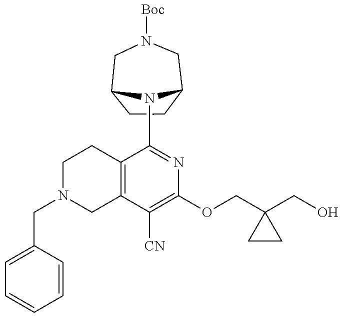 [SMILES: CC(C)(C)OC(=O)N1C[C@H]2CCC[C@@H](C1)N2c1nc(OCC2(CO)CC2)c(C#N)c2c1CCN(Cc1ccccc1)C2] | Int-4A |
| 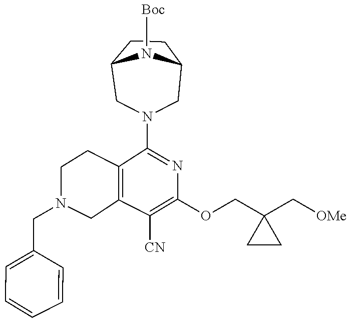 [SMILES: COCC1(COc2nc(N3C[C@H]4CCC[C@@H](C3)N4C(=O)OC(C)(C)C)c3c(c2C#N)CN(Cc2ccccc2)CC3)CC1] | Int-5 |
| 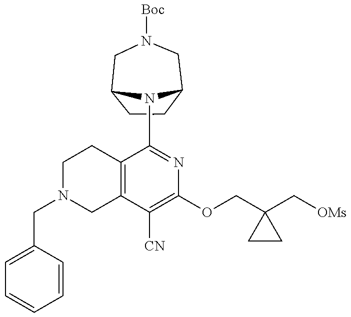 [SMILES: CC(C)(C)OC(=O)N1C[C@H]2CCC[C@@H](C1)N2c1nc(OCC2(COS(C)(=O)=O)CC2)c(C#N)c2c1CCN(Cc1ccccc1)C2] | Int-5A |
| 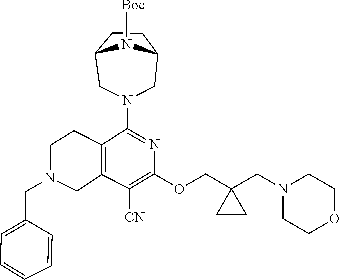 [SMILES: CC(C)(C)OC(=O)N1[C@@H]2CCC[C@H]1CN(c1nc(OCC3(CN4CCOCC4)CC3)c(C#N)c3c1CCN(Cc1ccccc1)C3)C2] | Int-6 |
| 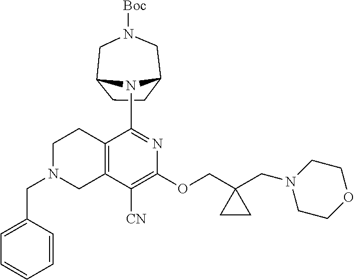 [SMILES: CC(C)(C)OC(=O)N1C[C@H]2CCC[C@@H](C1)N2c1nc(OCC2(CN3CCOCC3)CC2)c(C#N)c2c1CCN(Cc1ccccc1)C2] | Int-6A |
| 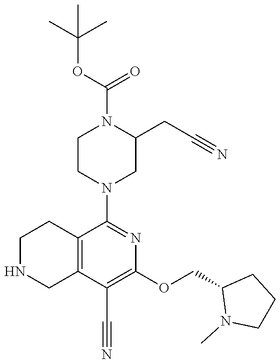 [SMILES: CN1CCC[C@H]1COc1nc(N2CCN(C(=O)OC(C)(C)C)C(CC#N)C2)c2c(c1C#N)CNCC2] | Int-6B |
| 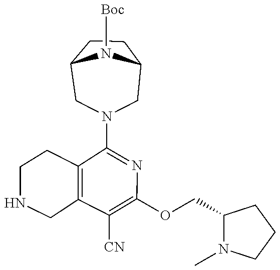 [SMILES: CN1CCC[C@H]1COc1nc(N2C[C@H]3CCC[C@@H](C2)N3C(=O)OC(C)(C)C)c2c(c1C#N)CNCC2] | Int-7 |
| 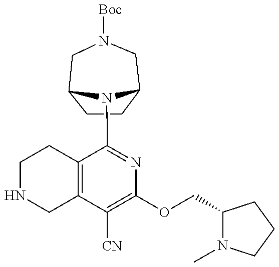 [SMILES: CN1CCC[C@H]1COc1nc(N2[C@@H]3CCC[C@H]2CN(C(=O)OC(C)(C)C)C3)c2c(c1C#N)CNCC2] | Int-7A |
| 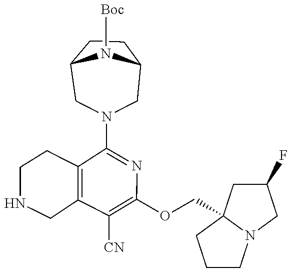 [SMILES: CC(C)(C)OC(=O)N1[C@@H]2CCC[C@H]1CN(c1nc(OC[C@@]34CCCN3C[C@H](F)C4)c(C#N)c3c1CCNC3)C2] | Int-8 |
| 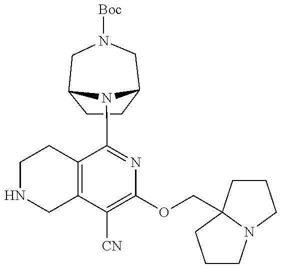 [SMILES: CC(C)(C)OC(=O)N1C[C@H]2CCC[C@@H](C1)N2c1nc(OCC23CCCN2CCC3)c(C#N)c2c1CCNC2] | Int-8A |
| 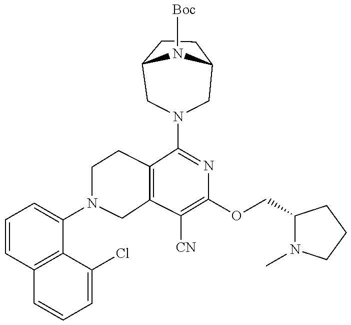 [SMILES: CN1CCC[C@H]1COc1nc(N2C[C@H]3CCC[C@@H](C2)N3C(=O)OC(C)(C)C)c2c(c1C#N)CN(c1cccc3cccc(Cl)c13)CC2] | Int-9 |
| 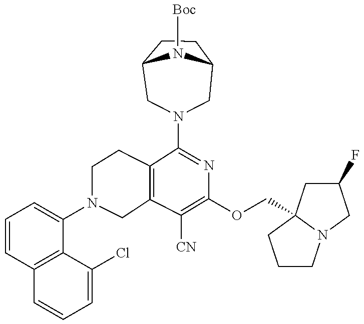 [SMILES: CC(C)(C)OC(=O)N1[C@@H]2CCC[C@H]1CN(c1nc(OC[C@@]34CCCN3C[C@H](F)C4)c(C#N)c3c1CCN(c1cccc4cccc(Cl)c14)C3)C2] | Int-10 |
| 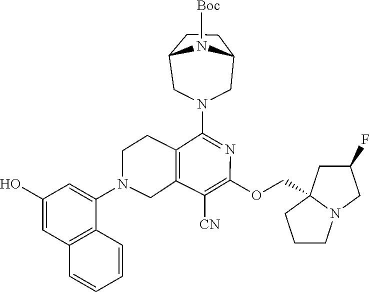 [SMILES: CC(C)(C)OC(=O)N1[C@@H]2CCC[C@H]1CN(c1nc(OC[C@@]34CCCN3C[C@H](F)C4)c(C#N)c3c1CCN(c1cc(O)cc4ccccc14)C3)C2] | Int-11 |
| 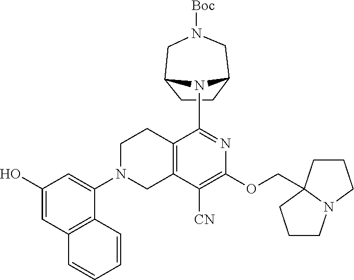 [SMILES: CC(C)(C)OC(=O)N1C[C@H]2CCC[C@@H](C1)N2c1nc(OCC23CCCN2CCC3)c(C#N)c2c1CCN(c1cc(O)cc3ccccc13)C2] | Int-11A |
| 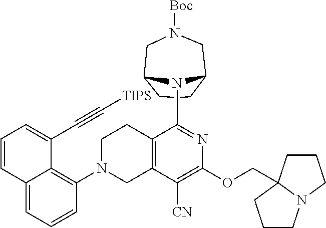 | Int-12A |
| 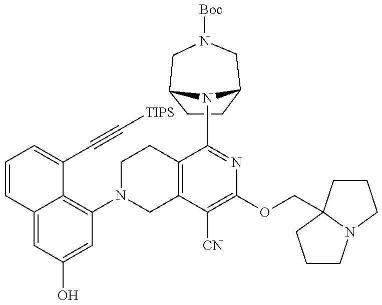 | Int-13A |
| 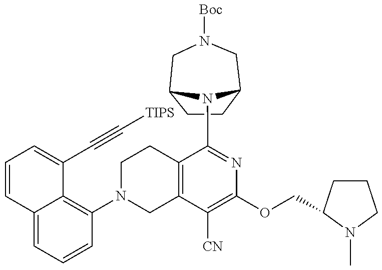 | Int-14A |
| 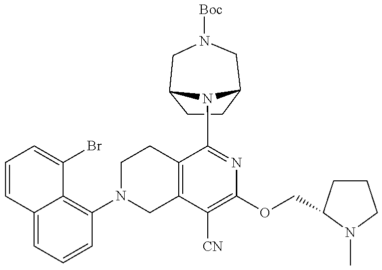 [SMILES: CN1CCC[C@H]1COc1nc(N2[C@@H]3CCC[C@H]2CN(C(=O)OC(C)(C)C)C3)c2c(c1C#N)CN(c1cccc3cccc(Br)c13)CC2] | Int-15A |
| 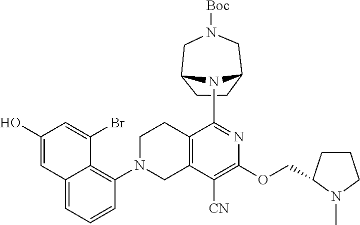 [SMILES: CN1CCC[C@H]1COc1nc(N2[C@@H]3CCC[C@H]2CN(C(=O)OC(C)(C)C)C3)c2c(c1C#N)CN(c1cccc3cc(O)cc(Br)c13)CC2] | Int-16A |
| 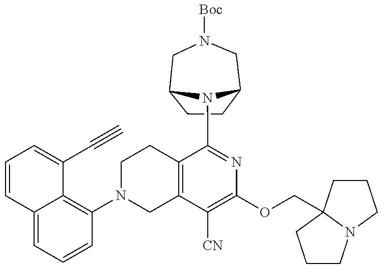 [SMILES: C#Cc1cccc2cccc(N3CCc4c(N5[C@@H]6CCC[C@H]5CN(C(=O)OC(C)(C)C)C6)nc(OCC56CCCN5CCC6)c(C#N)c4C3)c12] | Int-17A |
| 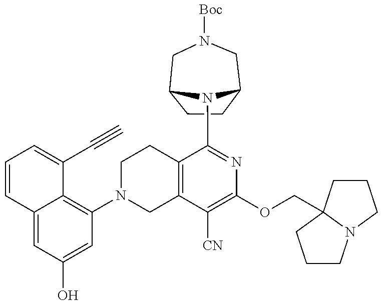 [SMILES: C#Cc1cccc2cc(O)cc(N3CCc4c(N5[C@@H]6CCC[C@H]5CN(C(=O)OC(C)(C)C)C6)nc(OCC56CCCN5CCC6)c(C#N)c4C3)c12] | Int-18A |
| 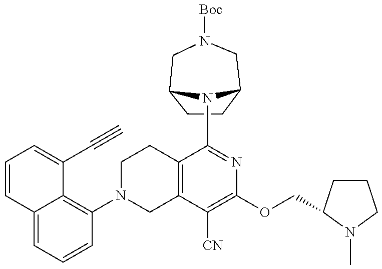 [SMILES: C#Cc1cccc2cccc(N3CCc4c(N5[C@@H]6CCC[C@H]5CN(C(=O)OC(C)(C)C)C6)nc(OC[C@@H]5CCCN5C)c(C#N)c4C3)c12] | Int-19A |
| 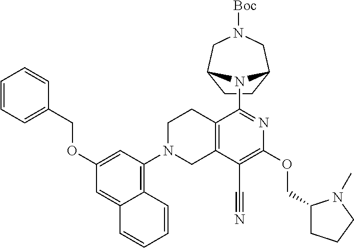 [SMILES: CN1CCC[C@@H]1COc1nc(N2[C@@H]3CC[C@H]2CN(C(=O)OC(C)(C)C)C3)c2c(c1C#N)CN(c1cc(OCc3ccccc3)cc3ccccc13)CC2] | Int-20A |
| 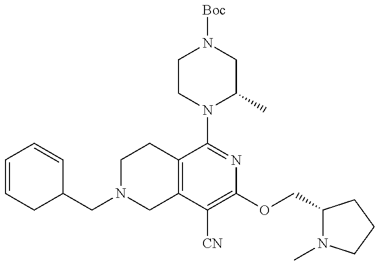 [SMILES: C[C@H]1CN(C(=O)OC(C)(C)C)CCN1c1nc(OC[C@@H]2CCCN2C)c(C#N)c2c1CCN(CC1C=CC=CC1)C2] | Int-21 |

Example 1: 1-(3,8-diazabicyclo[3.2.1]octan-3-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile, hydrochloride: To a cooled solution of tert-butyl 3-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate (0.057 g) in dichloromethane (2 ml) was added dioxane:HCl (1 ml), stirred reaction mixture for 1 hr at 0° C. After completion of the reaction, it was evaporated under reduced pressure to get oily compound, which was triturated using di ethyl ether to get solid as HCl salt. MS (m/z): 554.37 [M+H]*. 1H-NMR (6 ppm, DMSOD6, 400 MHz): 11.00 (bs, 1H), 9.87 (bs, 1H), 9.26 (bs, 1H), 8.01-7.99 (m, 1H), 7.69-7.67 (m, 1H), 7.42-7.36 (m, 1H), 7.29-7.22 (m, 1H), 6.92-6.89 (m, 1H), 6.84-6.83 (m, 1H), 4.73-4.65 (m, 4H), 3.89-3.86 (m, 2H), 3.50 (s, 3H), 3.40-3.35 (m, 1H), 3.26-3.09 (m, 5H), 2.90-2.25 (m, 4H), 2.20-2.25 (m, 1H), 2.15-2.05 (m, 6H), 1.87-1.80 (m, 1H).

Example 2: tert-butyl4-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate: A solution of tert-butyl 4-(4-cyano-3-(((S)-1-methylpyrrolidin-2-yl)methoxy)-5,6,7,8-tetrahydro-2,6-naphthyridin-1-yl)-2-(cyanomethyl) piperazine-1-carboxylate (Intermediate 5a, 1 gm, 0.002 mol), 1-bromo-8-chloronaphthalene (0.45 gm, 0.004 mol), Xantphos (0.23 gm, 0.0004 mol) and Cs2CO3 (1.96 gm, 0.006 mol) in Toluene (20 mL) was degassed with N2 for 20 minutes. Then Pd2(dba)3 (0.18 gm, 0.0002 mol) was added to the above solution and entire RM was stirred at 120° C. for 12 hrs. The reaction completion was checked by TLC using 5% MeOH in DCM and 1 drop of methanolic ammonia. After completion of reaction, the mixture was poured in cold water (20 mL) and extracted with ethyl acetate (30 mL×2). The organic layer was washed with brine solution (10 mL), dried over Na2SO4 and evaporated under reduced pressure to get a crude residue which was purified by column chromatography (100-200 mesh silica gel) using 3% MeOH in DCM as mobile phase. The pure fractions were collected and concentrated to get the desired product (0.7 gm, 63%). 1H-NMR (6 ppm, CDCl3, 400 MHz): 7.77-7.75 (m, 1H), 7.66-7.62 (m, 1H), 7.53-7.51 (m, 1H), 7.48-7.42 (m, 1H), 7.40-7.34 (m, 1H), 7.28-7.18 (m, 1H), 4.59-4.55 (m, 2H), 4.43-4.30 (m, 2H), 3.90-3.34 (m, 3H), 3.32-3.19 (m, 1H), 3.19-3.11 (m, 1H), 3.09-3.07 (m, 4H), 3.06-3.05 (m, 1H), 2.73-2.64 (m, 3H), 2.54 (s, 3H), 2.3-2.31 (m, 1H), 2.06-2.03 (m, 1H), 1.85-1.81 (m, 5H), 1.51 (s, 9H). MS (m/z): 656.3 (M+1). HPLC: 99.16%.

Example 3: 6-(8-chloronaphthalen-1-yl)-1-(3-(cyanomethyl)piperazin-1-yl)-3-(((S)-1-methylpyrrolidin-2-yl)methoxy)-5,6,7,8-tetrahydro-2,6-naphthyridine-4-carbonitrile hydrochloride: To a cooled solution of Example 2 (0.100 g) in dichloromethane (2 ml) was added dioxane:HCl (1 ml), stirred reaction mixture for 1 hr at 0° C. After completion of the reaction, it was evaporated under reduced pressure to get oily compound, which was triturated using di ethyl ether to get solid as HCl salt. MS (m/z): 556[M+H]⁺.

Example—4: tert-butyl (1R,5S)-8-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate: To a solution tert-butyl (1R,5S)-8-(4-cyano-3-(((S)-1-methylpyrrolidin-2-yl)methoxy)-5,6,7,8-tetrahydro-2,6-naphthyridin-1-yl)-3, 8-diazabicyclo[3.2.1]octane-3-carboxylate and 4-bromonaphthalen-2-ol in Toluene. Cs₂CO₃ was added, N2 gas purged for 10 min. To it Xanthphos and Pd2(dba)3 was added and again purged for 5 min. Reaction mixture was heated at 110° C. for 16 h. Reaction mixture diluted with water and extracted with (3×50 ml) EtOAc, combined organic layer washed with brine and dried over Na₂SO₄, evaporated under vacuum. Purification done by column chromatography, eluted at 2% DCM-Methanol to get tert-butyl (1R,5S)-8-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate. Mass: 625.3 [M−1].

Example—5: 1-((1R,5S)-3,8-diazabicyclo[3.2.1]octan-8-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl (1R,5S)-8-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate in DCM (ml) was cooled at 0 c. To it 4 M HCl in dioxane ( ) was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 9.81 (s, 1H), 7.87-7.86 (m, 1H), 7.76-7.71 (m, 1H), 7.61-7.55 (m, 2H), 7.42-7.39 (m, 2H), 4.85 (s, 2H), 4.63-4.61 (m, 1H), 4.58-4.45 (m, 2H), 3.98-3.96 (m, 1H), 3.87--3.86 (m, 1H), 3.76-3.72 (m, 1H), 3.60-3.59 (m, 4H), 3.27-3.25 (m, 2H), 3.14 (s, 3H), 2.76-2.73 (m, 1H), 2.44-2.36 (m, 2H), 2.25-2.05 (m, 4H), 1.16-1.14 (m, 4H). Mass: 525.2 [m+1].

Example—6: 1-((1R,5S)-3,8-diazabicyclo[3.2.1]octan-8-yl)-6-(3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile TFA salt: To a solution of tert-butyl (1R,5S)-8-(4-cyano-6-(3-hydroxynaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DCM (ml) was cooled at 0 c. To it TFA was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile TFA salt. 1H NMR: (MeOD 400 MHz): 9.81 (s, 1H), 7.87-7.86 (m, 1H), 7.76-7.71 (m, 1H), 7.61-7.55 (m, 2H), 7.42-7.39 (m, 2H), 5.69 (s, 2H), 4.12-3.99 (m, 2H), 3.97-3.94 (m, 2H), 3.79 (s, 1H), 3.86-3.64 (m, 4H), 3.52-3.48 (m, 4H), 2.99-2.97 (m, 4H), 1.19-1.18 (m, 6H), 1.16-1.14 (m, 6H). Mass: 551.3 [m+1].

Example—7: 1-((1R,5S)-3,8-diazabicyclo[3.2.1]octan-8-yl)-6-(8-bromonaphthalen-1-yl)-3-((tetrahydro-1H-pyrrolizin-7a(5H)-yl)methoxy)-5,6,7,8-tetrahydro-2,6-naphthyridine-4-carbonitrile TFA salt: To a solution of tert-butyl (1R,5S)-8-(6-(8-bromonaphthalen-1-yl)-4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DCM (ml) was cooled at 0 c. To it TFA was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-bromonaphthalen-1-yl)-3-((tetrahydro-1H-pyrrolizin-7a(5H)-yl)methoxy)-5,6,7,8-tetrahydro-2,6-naphthyridine-4-carbonitrile TFA salt. 1HNNMR: (MeOD 400 MHz): 7.91-7.89 (m, 1H), 7.83-7.81 (m, 1H), 7.75-7.73 (m, 1H), 7.55-7.51 (m, 1H), 7.41-7.40 (m, 1H), 7.33-7.29 (m, 1H), 5.69 (s, 2H), 4.12-3.99 (m, 2H), 3.97-3.94 (m, 2H), 3.79 (s, 1H), 3.86-3.64 (m, 4H), 3.52-3.48 (m, 4H), 2.99-2.97 (m, 4H), 1.19-1.18 (m, 6H), 1.16-1.14 (m, 6H). Mass: 61[m+1].

Example—8: 1-((1R,5S)-3,8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile TFA Salt: To a solution oftert-butyl (1R,5S)-8-(4-cyano-6-(8-ethynylnaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DCM was cooled at 0 c. To it TFA was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile TFA salt. 1H NMR: (MeOD 400 MHz): 7.87-7.86 (m, 1H), 7.75-7.73 (m, 1H), 7.58-7.50 (m, 2H), 7.41-7.36 (m, 2H), 5.68 (s, 2H), 4.12-3.98 (m, 2H), 3.97-3.95 (m, 2H), 3.79 (s, 1H), 3.86-3.65 (m, 4H), 3.51-3.48 (m, 4H), 2.98-2.97 (m, 4H), 1.19-1.17 (m, 6H), 1.16-1.14 (m, 6H). Mass: 559.3 [m+1].

Example—9: 1-((1R,5S)-3,8-diazabicyclo[3.2.1]octan-8-yl)-6-(8-bromo-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride: To a solution of tert-butyl (1R,5S)-8-(6-(8-bromo-3-hydroxynaphthalen-1-yl)-4-cyano-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-bromo-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride. 1H NMR: (MeOD 400 MHz): 9.81 (s, 1H), 7.87-7.86 (m, 1H), 7.76-7.71 (m, 1H), 7.61-7.55 (m, 2H), 7.42-7.39 (m, 1H), 5.69 (s, 2H), 4.12-3.99 (m, 2H), 3.97-3.94 (m, 2H), 3.79 (s, 1H), 3.86-3.64 (m, 4H), 3.52-3.48 (m, 4H), 2.99-2.97 (m, 4H), 1.19-1.18 (m, 6H), 1.16-1.14 (m, 6H). Mass: 628.5 [m+1].

Example—10: 1-((1R,5S)-3,8-diazabicyclo[3.2.1]octan-8-yl)-6-(8-bromonaphthalen-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl (1R,5S)-8-(6-(8-bromonaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-bromonaphthalen-1-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 7.91-7.89 (m, 1H), 7.83-7.81 (m, 1H), 7.75-7.73 (m, 1H), 7.55-7.51 (m, 1H), 7.41-7.40 (m, 1H), 7.33-7.29 (m, 1H), 4.85 (s, 2H), 4.63-4.61 (m, 1H), 4.58-4.45 (m, 2H), 3.98-3.96 (m, 1H), 3.87--3.86 (m, 1H), 3.76-3.72 (m, 1H), 3.60-3.59 (m, 4H), 3.27-3.25 (m, 2H), 3.14 (s, 3H), 2.76-2.73 (m, 1H), 2.44-2.36 (m, 2H), 2.25-2.05 (m, 4H), 1.16-1.14 (m, 4H). Mass: 588.3 [m+1].

Example—11: 1-((1R,5S)-3,8-diazabicyclo[3.2.1]octan-8-yl)-6-(8-ethynylnaphthalen-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl (1R,5S)-8-(4-cyano-6-(8-ethynylnaphthalen-1-yl)-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-ethynylnaphthalen-1-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 7.87-7.86 (m, 1H), 7.76-7.71 (m, 1H), 7.61-7.55 (m, 2H), 7.42-7.39 (m, 2H), 4.89 (s, 2H), 3.79 (s, 1H), 3.76-3.65 (m, 4H), 3.51-3.48 (m, 6H), 2.98-2.97 (m, 5H), 2.79 (s, 3H), 2.44-2.43 (m, 2H), 1.17-1.14 (m, 6H). Mass: 533.3 [m+1].

Example—12: 1-((1R,5S)-3,8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride: To a solution of tert-butyl (1R,5S)-8-(4-cyano-6-(8-ethynylnaphthalen-1-yl)-3-((tetrahydro-1H-pyrrolizin-7a(5H)-yl)methoxy)-5,6,7,8-tetrahydro-2,6-naphthyridin-1-yl)-3, 8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride. 1H NMR: (MeOD 400 MHz): 7.87-7.86 (m, 1H), 7.75-7.73 (m, 1H), 7.58-7.50 (m, 2H), 7.41-7.36 (m, 2H), 5.68 (s, 2H), 4.12-3.98 (m, 2H), 3.97-3.95 (m, 2H), 3.79 (s, 1H), 3.86-3.65 (m, 4H), 3.51-3.48 (m, 4H), 2.98-2.97 (m, 4H), 1.19-1.17 (m, 6H), 1.16-1.14 (m, 6H). Mass: 559.3 [m+1].

Example—13: 6-benzyl-1-((1R,5S)-3,8-diazabicyclo[3.2.1]octan-8-yl)-3-((1-(morpholinomethyl)cyclopropyl)methoxy)-5,6,7,8-tetrahydro-2,6-naphthyridine-4-carbonitrile Hydrochloride: To a solution of tert-butyl (1R,5S)-8-(6-benzyl-4-cyano-3-((1-(morpholinomethyl)cyclopropyl)methoxy)-5,6,7,8-tetrahydro-2,6-naphthyridin-1-yl)-3, 8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 6-benzyl-1-((1R,5S)-3,8-diazabicyclo[3.2.1]octan-8-yl)-3-((1-(morpholinomethyl)cyclopropyl)methoxy)-5,6,7,8-tetrahydro-2,6-naphthyridine-4-carbonitrile Hydrochloride. 1H NMR: (MeOD 400 MHz): 7.66-7.55 (m, 5H), 4.49 (s, 2H), 3.89 (s, 2H), 3.78 (s, 2H), 3.74-3.65 (m, 4H), 3.19-3.01 (m, 4H), 2.98-2.86 (m, 6H), 2.39-2.35 (m, 4H), 2.31 (s, 2H), 1.17-1.15 (m, 4H), 0.86-0.45 (m, 4H). Mass: 529.3 [m+1].

Example—14: 1-((1R,5S)-3,8-diazabicyclo[3.2.1]octan-8-yl)-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride: To a solution of tert-butyl (1R,5S)-8-(4-cyano-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridin-1-yl)-3,8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile Hydrochloride. 1H NMR: (MeOD 400 MHz): 9.81 (s, 1H), 7.87-7.86 (m, 1H), 7.75-7.73 (m, 1H), 7.58-7.50 (m, 2H), 7.41-7.36 (m, 2H), 5.68 (s, 2H), 4.12-3.98 (m, 2H), 3.97-3.95 (m, 2H), 3.79 (s, 1H), 3.86-3.65 (m, 4H), 3.51-3.48 (m, 4H), 2.98-2.97 (m, 4H), 1.19-1.17 (m, 6H), 1.16-1.14 (m, 6H). Mass: 576.1 [m+1].

Example—15: 1-((1R,5S)-3,8-diazabicyclo[3.2.1]octan-8-yl)-6-(8-bromo-3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl (1R,5S)-8-(6-(8-bromo-3-hydroxynaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-3-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-((1R,5S)-3,8-diazabicyclo[3.2.1]octan-8-yl)-6-(8-bromo-3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride. 1HNMR: (MeOD 400 MHz):): 9.81 (s, 1H), 7.87-7.86 (m, 1H), 7.76-7.71 (m, 1H), 7.61-7.55 (m, 2H), 7.42-7.39 (m, 1H), 4.99 (s, 2H), 3.98-3.65 (m, 4H), 3.51-3.48 (m, 6H), 2.98-2.97 (m, 5H), 2.79 (s, 3H), 2.44-2.43 (m, 2H), 1.17-1.14 (m, 6H). Mass: 604.2 [m+1].

Example—16: 1-(3,8-diazabicyclo[3.2.1]octan-3-yl)-6-(8-chloronaphthalen-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl 3-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-3,8-diazabicyclo[3.2.1]octane-8-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-(3,8-diazabicyclo[3.2.1]octan-3-yl)-6-(8-chloronaphthalen-1-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 7.87-7.86 (m, 1H), 7.75-7.73 (m, 1H), 7.58-7.50 (m, 2H), 7.41-7.36 (m, 2H), 4.58 (s, 2H), 3.89-3.86 (m, 2H), 3.65-3.62 (m, 2H), 3.46-3.31 (m, 4H), 3.26-3.03 (m, 2H), 2.98-2.96 (m, 1H), 2.86-2.84 (m, 2H), 2.56-2.53 (m, 2H), 2.44 (s, 3H), 2.39-2.26 (m, 3H), 1.18-1.16 (m, 3H), 1.14-1.12 (m, 3H). Mass: 542.7 [m+1].

Example—17: 1-(3,8-diazabicyclo[3.2.1]octan-3-yl)-6-(8-chloronaphthalen-1-yl)-3-(((2R)-2-fluorotetrahydro-1H-pyrrolizin-7a(5H)-yl)methoxy)-5,6,7,8-tetrahydro-2,6-naphthyridine-4-carbonitrile Hydrochloride: To a solution of tert-butyl 3-(6-(8-chloronaphthalen-1-yl)-4-cyano-3-(((2R)-2-fluorotetrahydro-1H-pyrrolizin-7a(5H)-yl)methoxy)-5,6,7,8-tetrahydro-2,6-naphthyridin-1-yl)-3, 8-diazabicyclo[3.2.1]octane-8-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-(3,8-diazabicyclo[3.2.1]octan-3-yl)-6-(8-chloronaphthalen-1-yl)-3-(((2R)-2-fluorotetrahydro-1H-pyrrolizin-7a(5H)-yl)methoxy)-5,6,7,8-tetrahydro-2,6-naphthyridine-4-carbonitrile Hydrochloride. 1H NMR: (MeOD 400 MHz): 7.88-7.85 (m, 1H), 7.74-7.72 (m, 1H), 7.57-7.50 (m, 2H), 7.42-7.36 (m, 2H), 5.68-5.54 (m, 1H), 4.85 (m, 4H), 4.67-4.59 (m, 2H), 4.19 (s, 2H), 4.05-3.94 (m, 4H), 3.51-3.48 (m, 3H), 2.98-2.97 (m, 2H), 2.74-2.61 (m, 2H), 2.44-2.43 (m, 3H), 2.39-2.26 (m, 3H), 1.17-1.15 (m, 2H). Mass: 586.9 [m+1].

Example—18: 6-benzyl-1-(3,8-diazabicyclo[3.2.1]octan-3-yl)-3-(((S)-1-methylpyrrolidin-2-yl)methoxy)-5,6,7,8-tetrahydro-2,6-naphthyridine-4-carbonitrile Hydrochloride: To a solution of tert-butyl 3-(6-benzyl-4-cyano-3-(((S)-1-methylpyrrolidin-2-yl)methoxy)-5,6,7,8-tetrahydro-2,6-naphthyridin-1-yl)-3, 8-diazabicyclo[3.2.1]octane-8-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 6-benzyl-1-(3,8-diazabicyclo[3.2.1]octan-3-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 7.66-7.55 (m, 5H), 4.59 (s, 2H), 3.86 (s, 2H), 3.65-3.64 (m, 2H), 3.51-3.48 (m, 4H), 2.98-2.97 (m, 2H), 2.89-2.86 (m, 1H), 2.84-2.82 (m, 2H), 2.65-2.63 (m, 2H), 2.56 (s, 3H), 2.39-2.26 (m, 3H), 1.18-1.16 (m, 3H), 1.14-1.12 (m, 3H). Mass: 473.1 [m+1].

Example—19: 6-benzyl-1-(3,8-diazabicyclo[3.2.1]octan-3-yl)-3-(((2R)-2-fluorotetrahydro-1H-pyrrolizin-7a(5H)-yl)methoxy)-5,6,7,8-tetrahydro-2,6-naphthyridine-4-carbonitrile Hydrochloride: To a solution of tert-butyl 3-(6-benzyl-4-cyano-3-(((2R)-2-fluorotetrahydro-1H-pyrrolizin-7a(5H)-yl)methoxy)-5,6,7,8-tetrahydro-2,6-naphthyridin-1-yl)-3,8-diazabicyclo[3.2.1]octane-8-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 6-benzyl-1-(3,8-diazabicyclo[3.2.1]octan-3-yl)-3-(((2R)-2-fluorotetrahydro-1H-pyrrolizin-7a(5H)-yl)methoxy)-5,6,7,8-tetrahydro-2,6-naphthyridine-4-carbonitrile Hydrochloride. 1H NMR: (MeOD 400 MHz): 7.66-7.55 (m, 5H), 4.59 (s, 2H), 4.56-4.51 (m, 2H), 4.40 (s, 2H), 4.19 (s, 2H), 4.05-3.94 (m, 4H), 3.75 (s, 2H), 3.50-3.47 (m, 3H), 2.98-2.97 (m, 2H), 2.73-2.60 (m, 3H), 2.45-2.44 (m, 3H), 2.37-2.25 (m, 4H), 1.16-1.15 (m, 2H). Mass: 516.9 [m+1].

Example—20: tert-butyl ((R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-methyl-1-oxobutan-2-yl)carbamate: To a solution of (S)-6-benzyl-3-((1-methylpyrrolidin-2-yl)methoxy)-1-(piperazin-1-yl)-5,6,7,8-tetrahydro-2,6-naphthyridine-4-carbonitrile Hydrochloride in THE (ml) was cooled at 0 c. To it DIPEA and (tert-butoxycarbonyl)-D-valine was added, stirred for 15 min. HOBT and DCC was added reaction mixture was stirred at RT for 16 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl ((R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-methyl-1-oxobutan-2-yl)carbamate. Mass: 646.4 [m+1].

Example—21: 1-(4-(D-valyl)piperazin-1-yl)-6-benzyl-3-(((S)-1-methylpyrrolidin-2-yl)methoxy)-5,6,7,8-tetrahydro-2,6-naphthyridine-4-carbonitrile Hydrochloride: To a solution of tert-butyl ((R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-methyl-1-oxobutan-2-yl)carbamate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 1-(4-(D-valyl)piperazin-1-yl)-6-benzyl-3-(((S)-1-methylpyrrolidin-2-yl)methoxy)-5,6,7,8-tetrahydro-2,6-naphthyridine-4-carbonitrile HydrochloridelH NMR: (MeOD 400 MHz): 7.66-7.55 (m, 5H), 4.84 (s, 2H), 3.89 (s, 2H), 3.86-3.82 (m, 4H), 3.79-3.75 (m, 2H), 3.65-3.61 (m, 3H), 2.96 (m, 2H), 2.85-2.79 (m, 3H), 2.45-2.44 (m, 2H), 2.39 (s, 3H), 2.21 (m, 1H), 1.16-1.13 (m, 4H), 1.01-0.99 (m, 6H). Mass: 547.0 [m+1].

Example—22: tert-butyl ((2S,3R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-(benzyloxy)-1-oxobutan-2-yl)carbamate: To a solution of (S)-6-benzyl-3-((1-methylpyrrolidin-2-yl)methoxy)-1-(piperazin-1-yl)-5,6,7,8-tetrahydro-2,6-naphthyridine-4-carbonitrile Hydrochloride in THE (ml) was cooled at 0 c. To it DIPEA and O-benzyl-N-(tert-butoxycarbonyl)-L-threonine was added, stirred for 15 min. HOBT and DCC was added reaction mixture was stirred at RT for 16 h. Reaction was poured into water and extracted with Ethyl acetate(3×), mix organic layer dried under Na₂SO₄ and evaporated under vacuum, crude was purified by column chromatography and eluted at (1:10) Methanol: DCM to get tert-butyl ((2S,3R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-(benzyloxy)-1-oxobutan-2-yl)carbamate. Mass: 738.6 [m+1].

Example—23: 6-benzyl-1-(4-(O-benzyl-L-threonyl)piperazin-1-yl)-3-(((S)-1-methylpyrrolidin-2-yl)methoxy)-5,6,7,8-tetrahydro-2,6-naphthyridine-4-carbonitrile Hydrochloride: To a solution of tert-butyl ((2S,3R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-(benzyloxy)-1-oxobutan-2-yl)carbamate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 6-benzyl-1-(4-(O-benzyl-L-threonyl)piperazin-1-yl)-3-(((S)-1-methylpyrrolidin-2-yl)methoxy)-5,6,7,8-tetrahydro-2,6-naphthyridine-4-carbonitrile Hydrochloride. 1H NMR: (MeOD 400 MHz): 7.81-7.79 (m, 5H), 7.66-7.61 (m, 5H), 4.84 (s, 2H), 4.69 (s, 2H), 3.89 (s, 2H), 3.86-3.82 (m, 4H), 3.81-3.80 (m, 2H), 3.79-3.75 (m, 4H), 3.65-3.63 (m, 3H), 2.97 (m, 2H), 2.85-2.79 (m, 3H), 2.45-2.44 (m, 2H), 2.38 (s, 3H), 1.16-1.13 (m, 4H), 1.01-0.99 (m, 3H). Mass: 638.0 [m+1].

Example—24: 6-benzyl-1-((1R,5S)-3,8-diazabicyclo[3.2.1]octan-3-yl)-3-((1-(morpholinomethyl)cyclopropyl)methoxy)-5,6,7,8-tetrahydro-2,6-naphthyridine-4-carbonitrile Hydrochloride: To a solution of tert-butyl (1R,5S)-3-(6-benzyl-4-cyano-3-((1-(morpholinomethyl)cyclopropyl)methoxy)-5,6,7,8-tetrahydro-2,6-naphthyridin-1-yl)-3, 8-diazabicyclo[3.2.1]octane-8-carboxylate in DCM was cooled at 0 c. To it 4 M HCl in dioxane was added. Reaction mixture was stirred at RT for 1 h. Reaction was evaporated under vacuum, crude was triturated with diethyl ether and dried under vacuum to get 6-benzyl-1-((1R,5S)-3,8-diazabicyclo[3.2.1]octan-3-yl)-3-((1-(morpholinomethyl)cyclopropyl)methoxy)-5,6,7,8-tetrahydro-2,6-naphthyridine-4-carbonitrile Hydrochloride. 1H NMR: (MeOD 400 MHz): 7.66-7.55 (m, 5H), 4.59 (s, 2H), 4.51 (s, 2H), 4.41 (s, 2H), 4.15 (s, 2H), 4.01-3.89 (s, 4H), 3.81-3.78 (m, 3H), 3.66 (s, 3H), 3.17-3.47 (m, 4H), 2.12 (m, 3H), 1.36-1.32 (m, 2H), 1.16-1.13 (m, 2H), 0.97-0.92 (m, 4H). Mass: 529.1 [m+1].

Example—25: 1-(3,8-diazabicyclo[3.2.1]octan-3-yl)-3-(((2R)-2-fluorotetrahydro-1H-pyrrolizin-7a(5H)-yl)methoxy)-6-(3-hydroxynaphthalen-1-yl)-5,6,7,8-tetrahydro-2,6-naphthyridine-4-carbonitrile Hydrochloride: To a solution of tert-butyl 3-(4-cyano-3-(((2R)-2-fluorotetrahydro-1H-pyrrolizin-7a(5H)-yl)methoxy)-6-(3-hydroxynaphthalen-1-yl)-5,6,7,8-tetrahydro-2,6-naphthyridin-1-yl)-3, 8-diazabicyclo[3.2.1]octane-8-carboxylate in DCM was cooled at 0 c. To it 4 M HCl in dioxane ( ) was added. Reaction mixture was stirred at RT for 1 h. Reaction was evaporated under vacuum, crude was triturated with diethyl ether and dried under vacuum to get 1-(3,8-diazabicyclo[3.2.1]octan-3-yl)-3-(((2R)-2-fluorotetrahydro-1H-pyrrolizin-7a(5H)-yl)methoxy)-6-(3-hydroxynaphthalen-1-yl)-5,6,7,8-tetrahydro-2,6-naphthyridine-4-carbonitrile Hydrochloride. 1H NMR: (MeOD 400 MHz): 8.05-8.03 (m, 1H), 7.65-7.63 (m, 1H), 7.39-7.23 (m, 2H), 7.10-7.08 (m, 1H), 6.81 (s, 1H), 5.68-5.55 (m, 1H), 4.85 (s, 2H), 4.67-4.59 (m, 2H), 4.40 (s, 2H), 4.18 (s, 2H), 4.05-3.95 (m, 4H), 3.50-3.47 (m, 3H), 2.98 (m, 2H), 2.73-2.60 (m, 2H), 2.45-2.44 (m, 3H), 2.38-2.25 (m, 4H), 1.16-1.15 (m, 2H). Mass: 569.8 [m+1].

Example—26 to 74: Aforementioned examples were synthesyzed using the respective intermediates as disclosed in PCT application No. PCT/B2022/059630 using the procedure wherein the solution of respective intermediates in dichloromethane or methanol was treated with either Trifluroacetic acid (TFA) or dioxane in HCl at 0° C. for 1-2 hr. The reaction completion was checked by TLC using Methanol: DCM (50%) and 1 drop of methanolic ammonia. The solvent was evaporated under reduced pressure to get the respective individual compounds. The characterization data is reported below in Table-4:

TABLE 4

| IUPAC name | Yeild | | Mass |
|---|---|---|---|
| Example-26: 3-(1,1-dioxidothiomorpholino)-6-(naphthalen-1- | | | 503.22[M + H]⁺ |
| yl)-1-(piperazin-1-yl)-5,6,7,8-tetrahydro-2,6-naphthyridine-4- | | | |
| carbonitrile Hydrochloride; | | | |
| Example-27: 6-(8-chloronaphthalen-1-yl)-3-morpholino-1- | | | 490.21[M + H]⁺ |
| (piperazin-1-yl)-5,6,7,8-tetrahydro-2,6-naphthyridine-4- | | | |
| carbonitrile Hydrochloride; | | | |
| Example-28: 3-(2-morpholinoethoxy)-6-(naphthalen-1-yl)-1- | | | 499.20[M + H]⁺ |
| (piperazin-1-yl)-5,6,7,8-tetrahydro-2,6-naphthyridine-4- | | | |
| carbonitrile Hydrochloride; | | | |
| Example-29: 6-(8-chloronaphthalen-1-yl)-3-(2- | | | 534.20[M + H]⁺ |
| morpholinoethoxy)-1-(piperazin-1-yl)-5,6,7,8-tetrahydro-2,6- | | | |
| naphthyridine-4-carbonitrile Hydrochloride; | | | |
| Example-30: 1-(2,6-dimethylmorpholino)-6-(naphthalen-1-yl)- | | | 483.30[M + H]⁺ |
| 3-(piperazin-1-yl)-5,6,7,8-tetrahydro-2,6-naphthyridine-4- | | | |
| carbonitrile Hydrochloride; | | | |
| Example-31: 6-(8-chloronaphthalen-1-yl)-1-(2,6- | | | 518.24[M + H]⁺ |
| dimethylmorpholino)-3-(piperazin-1-yl)-5,6,7,8-tetrahydro- | | | |
| 2,6-naphthyridine-4-carbonitrile Hydrochloride; | | | |
| Example-32: 3-(3-(cyanomethyl)piperazin-1-yl)-1-(2,6- | | | 522.29[M + H]⁺ |
| dimethylmorpholino)-6-(naphthalen-1-yl)-5,6,7,8-tetrahydro- | | | |
| 2,6-naphthyridine-4-carbonitrile Hydrochloride; | | | |
| Example-33: (S)-6-(8-chloronaphthalen-1-yl)-3-((1- | | | 518.30[M + H]⁺ |
| methylpyrrolidin-2-yl)methoxy)-1-(piperazin-1-yl)-5,6,7,8- | | | |
| tetrahydro-2,6-naphthyridine-4-carbonitrile Hydrochloride; | | | |
| Example-34: (S)-6-(3-hydroxynaphthalen-1-yl)-3-((1- | | | 499.28[M + H]⁺ |
| methylpyrrolidin-2-yl)methoxy)-1-(piperazin-1-yl)-5,6,7,8- | | | |
| tetrahydro-2,6-naphthyridine-4-carbonitrile Hydrochloride; | | | |
| Example-35: (S)-3-((1-methylpyrrolidin-2-yl)methoxy)-1- | | | 485.27[M + H]⁺ |
| (piperazin-1-yl)-6-(quinazolin-4-yl)-5,6,7,8-tetrahydro-2,6- | | | |
| naphthyridine-4-carbonitrile Hydrochloride; | | | |
| Example-36: 6-(8-chloronaphthalen-1-yl)-1-(3- | | | 531.30[M + H]⁺ |
| methylpiperazin-1-yl)-3-(((S)-1-methylpyrrolidin-2- | | | |
| yl)methoxy)-5,6,7,8-tetrahydro-2,6-naphthyridine-4- | | | |
| carbonitrile Hydrochloride; | | | |
| Example-37: 1-(3-(cyanomethyl)piperazin-1-yl)-3-(2,6- | | | 522.29[M + H]⁺ |
| dimethylmorpholino)-6-(naphthalen-1-yl)-5,6,7,8-tetrahydro- | | | |
| 2,6-naphthyridine-4-carbonitrile Hydrochloride; | | | |
| Example-38: 1-((S)-3-(cyanomethyl)piperazin-1-yl)-3-(2,6- | | | 522.29[M + H]⁺ |
| dimethylmorpholino)-6-(naphthalen-1-yl)-5,6,7,8-tetrahydro- | | | |
| 2,6-naphthyridine-4-carbonitrile Hydrochloride; | | | |
| Example-39: 1-((R)-3-(cyanomethyl)piperazin-1-yl)-3-(2,6- | | | 522.29[M + H]⁺ |
| dimethylmorpholino)-6-(naphthalen-1-yl)-5,6,7,8-tetrahydro- | | | |
| 2,6-naphthyridine-4-carbonitrile Hydrochloride; | | | |
| Example-40: 1-(3-(cyanomethyl)piperazin-1-yl)-3-(((S)-1- | | | 522.29[M + H]⁺ |
| methylpyrrolidin-2-yl)methoxy)-6-(naphthalen-1-yl)-5,6,7,8- | | | |
| tetrahydro-2,6-naphthyridine-4-carbonitrile Hydrochloride; | | | |
| Example-41: 1-((S)-3-(cyanomethyl)piperazin-1-yl)-3-(((S)-1- | | | 522.29[M + H]⁺ |
| methylpyrrolidin-2-yl)methoxy)-6-(naphthalen-1-yl)-5,6,7,8- | | | |
| tetrahydro-2,6-naphthyridine-4-carbonitrile Hydrochloride; | | | |
| Example-42: 1-((R)-3-(cyanomethyl)piperazin-1-yl)-3-(((S)-1- | | | 522.29[M + H]⁺ |
| methylpyrrolidin-2-yl)methoxy)-6-(naphthalen-1-yl)-5,6,7,8- | | | |
| tetrahydro-2,6-naphthyridine-4-carbonitrile Hydrochloride; | | | |
| Example-43: 1-(3-(cyanomethyl)piperazin-1-yl)-6-(3- | | | 538.40[M + H]⁺ |
| hydroxynaphthalen-1-yl)-3-(((S)-1-methylpyrrolidin-2- | | | |
| yl)methoxy)-5,6,7,8-tetrahydro-2,6-naphthyridine-4- | | | |
| carbonitrile Hydrochloride; | | | |
| Example-44: (S)-6-(isoquinolin-4-yl)-3-((1-methylpyrrolidin- | | | 484.12[M + H]⁺ |
| 2-yl)methoxy)-1-(piperazin-1-yl)-5,6,7,8-tetrahydro-2,6- | | | |
| naphthyridine-4-carbonitrile Hydrochloride; | | | |
| Example-45: (S)-3-((1-methylpyrrolidin-2-yl)methoxy)-1- | | | 484.12[M + H]⁺ |
| (piperazin-1-yl)-6-(quinolin-8-yl)-5,6,7,8-tetrahydro-2,6- | | | |
| naphthyridine-4-carbonitrile Hydrochloride; | | | |
| Example-46: 6-(8-chloronaphthalen-1-yl)-1-(3- | | | 556.30[M + H]⁺ |
| (cyanomethyl)piperazin-1-yl)-3-(((S)-1-methylpyrrolidin-2- | | | |
| yl)methoxy)-5,6,7,8-tetrahydro-2,6-naphthyridine-4- | | | |
| carbonitrile Hydrochloride; | | | |
| Example-47: 6-(8-chloronaphthalen-1-yl)-1-((S)-3- | | | 556.30[M + H]⁺ |
| (cyanomethyl)piperazin-1-yl)-3-(((S)-1-methylpyrrolidin-2- | | | |
| yl)methoxy)-5,6,7,8-tetrahydro-2,6-naphthyridine-4- | | | |
| carbonitrile Hydrochloride; | | | |
| Example-48: 6-(8-chloronaphthalen-1-yl)-1-((S)-3- | | | 556.30[M + H]⁺ |
| (cyanomethyl)piperazin-1-yl)-3-(((S)-1-methylpyrrolidin-2- | | | |
| yl)methoxy)-5,6,7,8-tetrahydro-2,6-naphthyridine-4- | | | |
| carbonitrile Hydrochloride; | | | |
| Example-49: 6-benzyl-3-chloro-1-(piperazin-1-yl)-5,6,7,8- | | | 368.16[M + H]⁺ |
| tetrahydro-2,6-naphthyridine-4-carbonitrile Hydrochloride; | | | |
| Example-50: 1-(4-aminopiperidin-1-yl)-6-benzyl-3-chloro- | | | 383.17[M + H]⁺ |
| 5,6,7,8-tetrahydro-2,6-naphthyridine-4-carbonitrile | | | |
| Hydrochloride; | | | |
| Example-51: 1-(4-aminopiperidin-1-yl)-6-benzyl-5,6,7,8- | | | 348.21[M + H]⁺ |
| tetrahydro-2,6-naphthyridine-4-carbonitrile Hydrochloride; | | | |
| Example-52: 6-benzyl-1,3-di(piperazin-1-yl)-5,6,7,8- | 0.5 | g | 419.32 [M + H]⁺ |
| tetrahydro-2,6-naphthyridine-4-carbonitrile Hydrochloride; | | | |
| Example-53: 6-benzyl-3-hydroxy-1-(piperazin-1-yl)-5,6,7,8- | 0.4 | g | 350.16 [M + H]⁺ |
| tetrahydro-2,6-naphthyridine-4-carbonitrile Hydrochloride; | | | |
| Example-54: 6-benzyl-3-morpholino-1-(piperazin-1-yl)- | 0.4 | g | 419.32 [M + H]⁺ |
| 5,6,7,8-tetrahydro-2,6-naphthyridine-4-carbonitrile | | | |
| Hydrochloride; | | | |
| Example-55: 6-benzyl-3-(4-methylpiperazin-1-yl)-1- | 0.5 | g | 469.12[M + H]⁺ |
| (piperazin-1-yl)-5,6,7,8-tetrahydro-2,6-naphthyridine-4- | | | |
| carbonitrile Hydrochloride; | | | |
| Example-56: 6-benzyl-1-(3-(cyanomethyl)piperazin-1-yl)-3- | 0.21 | g | 471.10 [M + H]⁺ |
| (4-methylpiperazin-1-yl)-5,6,7,8-tetrahydro-2,6- | | | |
| naphthyridine-4-carbonitrile Hydrochloride; | | | |
| Example-57: 6-(1-naphthoyl)-1-(3-(cyanomethyl)piperazin-1- | 0.2 | g | 535.12[M + H]⁺ |
| yl)-3-(4-methylpiperazin-1-yl)-5,6,7,8-tetrahydro-2,6- | | | |
| naphthyridine-4-carbonitrile Hydrochloride; | | | |
| Example-58: (S)-6-benzyl-1-(2-methylpiperazin-1-yl)-3-(4- | 0.75 | g | 447.12[M + H]⁺ |
| methylpiperazin-1-yl)-5,6,7,8-tetrahydro-2,6-naphthyridine-4- | | | |
| carbonitrile Hydrochloride; | | | |
| Example-59: (R)-6-benzyl-1-(2-methylpiperazin-1-yl)-3-(4- | 1.1 | g | 447.12[M + H]⁺ |
| methylpiperazin-1-yl)-5,6,7,8-tetrahydro-2,6-naphthyridine-4- | | | |
| carbonitrile Hydrochloride; | | | |
| Example-60: 3-(4-methylpiperazin-1-yl)-6-(naphthalen-1-yl)- | 0.4 | g | 468.00[M + H]⁺ |
| 1-(piperazin-1-yl)-5,6,7,8-tetrahydro-2,6-naphthyridine-4- | | | |
| carbonitrile Hydrochloride; | | | |
| Example-61: 6-benzyl-1-(piperazin-1-yl)-3-(piperidin-1-yl)- | 0.4 | g | 417.00[M + H]⁺ |
| 5,6,7,8-tetrahydro-2,6-naphthyridine-4-carbonitrile | | | |
| Hydrochloride; | | | |
| Example-62: 6-benzyl-3-(2-fluoropyridin-4-yl)-1-(piperazin-1- | 0.4 | g | 429.00[M + H]⁺ |
| yl)-5,6,7,8-tetrahydro-2,6-naphthyridine-4-carbonitrile; | | | |
| Example-63: 6-(naphthalen-1-yl)-1-(piperazin-1-yl)-3- | 0.28 | g | 453.00[M + H]⁺ |
| (piperidin-1-yl)-5,6,7,8-tetrahydro-2,6-naphthyridine-4- | | | |
| carbonitrile Hydrochloride; | | | |
| Example-64: 6-benzyl-3-((1-methylpyrrolidin-2-yl)methoxy)- | 0.35 | g | 447.00[M + H]⁺ |
| 1-(piperazin-1-yl)-5,6,7,8-tetrahydro-2,6-naphthyridine-4- | | | |
| carbonitrile Hydrochloride; | | | |
| Example-65: 6-(naphthalen-1-yl)-1,3-di(piperazin-1-yl)- | 0.5 | g | 419.32[M + H]⁺ |
| 5,6,7,8-tetrahydro-2,6-naphthyridine-4-carbonitrile | | | |
| Hydrochloride; | | | |
| Example-66: 6-(benzo[b]thiophen-4-yl)-1,3-di(piperazin-1-yl)- | 0.27 | g | 460.00[M + H]⁺ |
| 5,6,7,8-tetrahydro-2,6-naphthyridine-4-carbonitrile | | | |
| dihydrochloride; | | | |
| Example-67: 3-morpholino-6-(naphthalen-1-yl)-1-(piperazin- | 0.4 | g | 455.20[M + H]⁺ |
| 1-yl)-5,6,7,8-tetrahydro-2,6-naphthyridine-4-carbonitrile | | | |
| Hydrochloride; | | | |
| Example-68: 3-(4-acetylpiperazin-1-yl)-6-(naphthalen-1-yl)-1- | 0.4 | g | 496.00[M + H]⁺ |
| (piperazin-1-yl)-5,6,7,8-tetrahydro-2,6-naphthyridine-4- | | | |
| carbonitrile Hydrochloride; | | | |
| Example-69: 3-(2,6-dimethylmorpholino)-6-(naphthalen-1-yl)- | 0.4 | g | 483.12[M + H]⁺ |
| 1-(piperazin-1-yl)-5,6,7,8-tetrahydro-2,6-naphthyridine-4- | | | |
| carbonitrile Hydrochloride; | | | |
| Example-70: 3-(4-(methylsulfonyl)piperazin-1-yl)-6- | 0.4 | g | 532.00[M + H]⁺ |
| (naphthalen-1-yl)-1-(piperazin-1-yl)-5,6,7,8-tetrahydro-2,6- | | | |
| naphthyridine-4-carbonitrile Hydrochloride; | | | |
| Example-71: 3-(4-ethylpiperazin-1-yl)-6-(naphthalen-1-yl)-1- | | | 482.30[M + H]⁺ |
| (piperazin-1-yl)-5,6,7,8-tetrahydro-2,6-naphthyridine-4- | | | |
| carbonitrile Hydrochloride; | | | |
| Example-72: (S)-3-((1-methylpyrrolidin-2-yl)methoxy)-6- | | | 483.17[M + H]⁺ |
| (naphthalen-1-yl)-1-(piperazin-1-yl)-5,6,7,8-tetrahydro-2,6- | | | |
| naphthyridine-4-carbonitrile Hydrochloride; | | | |
| Example-73: 6-(naphthalen-1-yl)-1-(piperazin-1-yl)-5,6,7,8- | | | 370.40[M + H]⁺ |
| tetrahydro-2,6-naphthyridine-4-carbonitrile Hydrochloride; | | | |
| Example-74: 6-(naphthalen-1-yl)-1-(piperazin-1-yl)-3-(4- | 0.4 | g | 483.12[M + H]⁺ |
| propionylpiperazin-1-yl)-5,6,7,8-tetrahydro-2,6- | | | |
| naphthyridine-4-carbonitrile Hydrochloride; | | | |

### Example—75: 6-benzyl-1-((S)-2-methylpiperazin-1-yl)-3-((1-methylpyrrolidin-2-yl)methoxy)-5,6,7,8-tetrahydro-2,6-naphthyridine-4-carbonitrile hydrochloride

To a cooled solution of Intermediate 21 (0.015 g) in dichloromethane (2 ml) was added dioxane: HCl (1 ml), stirred reaction mixture for 1 hr at 0° C. After completion of the reaction, it was evaporated under reduced pressure to get oily compound, which was triturated using di ethyl ether to get solid as HCl salt. MS (m/z): 462.20 [M+H]⁺.

### Part C: Biological Activity

The biological and/or pharmacological properties of the compounds of this invention may be confirmed by variety of assays. The biological and/or pharmacological assays which can be carried out with the compounds according to the invention and/or their pharmaceutically acceptable salts is exemplified below.

### Test 1: In Vitro Cell Proliferation Assay in HPAC and AGS Cell Lines

Growth inhibition assays was carried out using 10% FBS supplemented media. Cells are to be seeded at a concentration of 2000-3,000 cells/well in a 96-well plate. Test compounds at a concentration range from 0.1 to 30 uM will be added after 24 hours. Growth will be assessed using the MTT or CCK-8 kit for measuring reduction at 0 h (prior to the addition of the test compound) and 72 to 96 hours after the addition of test compound. Absorbance read on a BIO-RAD iMark Microplate or any equivalent micloplate reader at a wavelength of 450 nm to 600 nm. Data will be analyzed and percent inhibition due to the test compound compared to the control is calculated accordingly.

TABLE 5

| % Inhibition of Cell Proliferation | |
|---|---|
| HPAC | AGS |

| Examples | TC1 | TC2 | TC1 | TC2 |
|---|---|---|---|---|
| Ex 1 | — | — | B | D |
| Ex 2 | — | — | — | — |
| Ex 3 | — | — | B | D |
| Ex 4 | — | — | D | D |
| Ex 5 | — | — | B | D |
| Ex 6 | A | C | — | — |
| Ex 7 | D | D | — | — |
| Ex 8 | A | C | — | — |
| Ex 9 | A | D | — | — |
| Ex 10 | D | D | — | — |
| Ex 11 | C | C | — | — |
| Ex 12 | C | C | — | — |
| Ex 13 | A | C | — | — |
| Ex 14 | A | C | — | — |
| Ex 15 | A | D | — | — |
| Ex 16 | C | D | C | C |
| Ex 17 | B | D | B | D |
| Ex 18 | A | B | B | D |
| Ex 19 | A | D | B | C |
| Ex 20 | A | B | — | — |
| Ex 21 | A | A | — | — |
| Ex 22 | — | — | — | — |
| Ex 23 | A | C | — | — |
| Ex 24 | A | B | — | — |
| Ex 25 | B | D | D | D |
| Ex 75 | A | A | A | A |

A = ≤25%;
B = >25 to ≤50;
C = >50 to ≤75;
D = >75% to 100%
TC1: Test Concentration of 3 uM & TC2: Test Concentration of 10 uM

Test 2: In Vitro Cell Proliferation Assay in KRASG12 Cell lines

Growth inhibition assays was carried out using 10% FBS supplemented media. Cells were seeded at a desired concentration of 1000-2,000 cells/well in a well plate with desired number of wells. Test compounds at a desired concentration range were added after 24 hours. Growth was assessed using the Cell Titer-Glo (CTG, Cell Signaling) for measuring reduction at 0 h (prior to the addition of the test compound) and 3 day (2D) or 7 day (3D) after the addition of test compound. Absorbance read on a BIO-RAD iMark Microplate or any equivalent microplate reader at a predetermined a wavelength for e.g. 450 nm. Data was be analysed and percent inhibition and/or IC50 for each test compound is calculated accordingly.

TABLE 6

| Example | Cell Line | IC50 |
|---|---|---|
| Ex16 | CT-26 | [symbol] [symbol] [symbol] [symbol] |
| Ex 17 | | [symbol] [symbol] [symbol] |
| Ex 25 | | [symbol] [symbol] |

[symbol]

[symbol]

= ≤10 to >5 uM; [symbol] [symbol] [symbol]
= ≤5 to >2.5 um; [symbol] [symbol] [symbol] [symbol]
= <2.5 um

### Test 3: Nucleotide Exchange Assay

HTRF-based nucleotide exchange assay detecting GTP binding to K-Ras

A human KRAS G12D protein (corresponding to amino acid 2-169) was mixed with a a-GST Tb antibody (1.5× solution) and 10 uL of the solution was added to the reaction wells). Compounds (each 10 concentrations or any other concentration of choice at 3-fold or fold of choice for serial dilutions with a starting concentration of ˜ 300 μm or or 100 μM or 50 uM) were then delivered to the reaction wells using acoustic dispenser (Echo, Labcyte) and incubated with the Kras/aGST-Tb antibody for 1-hour at room temperature. After 1-hour incubation 5 μL of SOS1/GTP solution (SOS1-(corresponding to amino acid 564-1049) and GDP-DY-647P1 prepared using reaction buffer-20 mM Hepes, pH7.4, 150 mM Nacl, 5 mM MgCl2, 1 mM DTT, 0.05% BSA, 0.0025% NP40) was added to reaction wells to initiate the exchange reaction. HTRF based SOS1 mediated exchange of GDP to GTP was measured on a microplate reader PEHRAstar (BMG Labtech) at an excitation wavelength of 337 nm and emission wavelengths of 665 and 620 nm. No—SOS1 reaction or highest control compound concentration was used as blank and % inhibition was calculated and/or IC50 was determined using Sigmoidal dose response (variable slope) equation.

TABLE 7

| Example | IC50 |
|---|---|
| Ex 16 | [symbol] [symbol] |
| Ex 17 | [symbol] [symbol] [symbol] [symbol] [symbol] |
| Ex 19 | [symbol] |
| Ex 24 | [symbol] |
| Ex 25 | [symbol] [symbol] [symbol] [symbol] [symbol] [symbol] |

[symbol]

= >1000 nM; [symbol] [symbol]
= <1000 to >200 nm; [symbol] [symbol] [symbol]
= <200 to >100 nm; [symbol] [symbol] [symbol] [symbol]
= >50 to ≤100 nM; [symbol] [symbol] [symbol] [symbol] [symbol]
= >25 to ≤50 nM; [symbol] [symbol] [symbol] [symbol] [symbol] [symbol]
= <25 nM

Test 4: Biomarker Evaluation in HPAC and AGS cells using Western blotting

Protein lysate were prepared using RIPA lysis buffer representing both the control and test samples. In the instant invention Protein lysate were prepared using HPAC and AGS cells treated with representative example of the invention over time course using a 9 point concentration-response for measuring modulation of pERK. Total Protein was estimated by Bradford method and absorbance is measured at 595 nm using Bio-Rad imark reader. The total proteins isolated were separated on a 10% SDS PAGE electrophoresis and transferred on to a Nitrocellulose membrane. After transfer, the membrane was blocked using 5% BSA prepared in PBST (0.1% tween-20) for 1 hr at room temperature and washed with 1× PBS and PBST. Membrane was then probed with Rabbit Monoclonal Primary antibody such as pERK (MA5-15173), ERK(MA5-15134), procured from Invitrogen™, ThermoFisher Scientific USA. Primary antibody (1:2000 dilution) prepared in 5% BSA, 0.1% tween-20 solution for overnight at 4° C. Following incubation with primary antibody membrane were washed thrice with 1×PBS and PBST followed by incubation with Goat Anti-Rabbit Secondary IgG HRP conjugated (from Invitrogen™) at (1:10000 dilutions, prepared in 3% skim milk, 0.1% tween-20) for 1 hr at room temperature. Following incubation, the blot is washed and developed using G-biosciences femto LUCENT™ PLUS-HRP Chemiluminescent reagent in the Bio-Rad Chemidoc™ Imager system. The resulted bands on the blot were quantified using the Image J software. Results: Representative compound tested demonstrated a dose and time dependent modulation of pERK, downstream biomarker representing KRAS signaling.

### Test 5: RAS Selectivity by Nucleotide Exchange Assay

HTRF-based nucleotide exchange assay detecting GTP binding to Ras proteins (H, N and K): A panel of human RAS protein such as KRASG12S, KRAS G12R, KRAS G12C, KRAS G13D, KRASQ61H, KRASWT, NRASWT, HRASWT were used to test selectivity of compounds of the invention, wherein each of the respective protein was mixed with a a-GST Tb antibody (1.5× solution) and 10 uL of the solution was added to the reaction wells). Compounds (each 10 concentrations or any other concentration of choice at 3-fold or fold of choice for serial dilutions with a starting concentration of ˜300 um or 100 μM or 50 uM) were then delivered to the reaction wells using acoustic dispenser (Echo, Labcyte) and incubated with the respective ras/aGST-Tb antibody for 1-hour at room temperature. After 1-hour incubation 5 μL of SOS1/GTP solution (SOS1-(corresponding to amino acid 564-1049) and GDP-DY-647P1 prepared using reaction buffer-20 mM Hepes, pH7.4, 150 mM Nacl, 5 mM MgCl2, 1 mM DTT, 0.05% BSA, 0.0025% NP40) was added to reaction wells to initiate the exchange reaction. HTRF based SOS1 mediated exchange of GDP to GTP was measured on a microplate reader PEHRAstar (BMG Labtech) at an excitation wavelength of 337 nm and emission wavelengths of 665 and 620 nm. No—SOS1 reaction or highest control compound concentration was used as blank and % inhibition was calculated and/or IC50 was determined using Sigmoidal dose response (variable slope) equation.

Results: Representative compound tested demonstrated a high degree of selectivity towards KRASG12D as comapraed to other RAS proteins tested with over 1500-to-10000-fold selectivity across the panel of protens been tested.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A compound of formula (A) 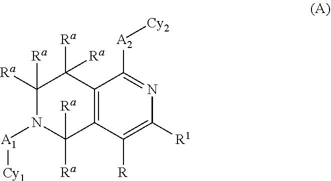
or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, O, S, —S(═O)ₚ—, —C(═O)—, —NR^{x}—, —CO—NR^{x}— and —NR^{x}—CO—;
A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, O, S, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G;
G is a group capable of forming an interaction with Aspartic acid at 12 position of KRAS protein;
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}—NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —N^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NbR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —N^{b}C(═O)OR^{c}, —NR^{b}C(═O)R^{c}, —NR^{b}C(═S)R^{c}, —NR^{b}C(═S)N^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRWS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbn atom may be joined to a form a Oxo (C═0), Imino (═NR^{b}), C═S(O)ₚ, or substituted or unsubstituted saturated or unsaturated 3-6 member ring, which may optionally include heteroatoms which may be same or different and are selected from O, NR^{a} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
Z′ is selected from O or S and
each occurrence of p is independently 0, 1 or 2.

2. The compound of claim 1, wherein A₂ is
(i) —CR^{b}R^{c}—; wherein each of R^{b} and R^{c} is independently selected from hydrogen, substituted or unsubstituted alkyl;
(ii) —CR^{b}R^{c}—; where in R^{b} is independently methyl or ethyl and R^{c} is hydrogen; or
(iii) absent.

3. The compound of anyone of claims 1-2, wherein R is Cyano (CN).

4. A compound of formula (A-I) 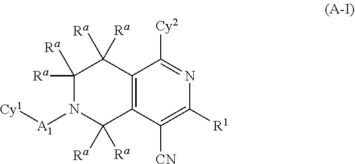
—or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by G;
G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R, —C(═O)NR^{b}R^{c}, —C(═O)ONR^{b}R^{c}, —NR^{b}R^{c}, —NR^{b}C(═O)NRR^{c}, —NR S(═O)R^{c}, —NR S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{c}R^{c}, —R^{b}NR^{c}C(═O)R^{b}, —R^{b}OR^{c}, —R^{b}C(═O)OR^{c}, —R^{c}C(═O)NR^{c}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}C(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{b} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2.

5. The compound of anyone of claims 1-4, wherein Cy² is selected from cyclic group selected from substituted or unsubstituted cycloalkyl, substituted or unsubstituted cycloalkylalkyl, substituted or unsubstituted heterocyclyl and substituted or unsubstituted heterocyclylalkyl, wherein each group is optionally further substituted with group G.

6. A compound of formula (A-II) 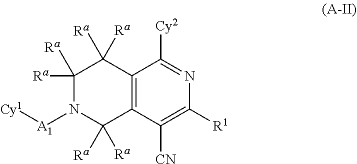
or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is substituted or unsubstituted heterocyclyl, which is optionally substituted by G;
G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R, —C(═O)NR^{b}R^{c}, —C(═O)ONR^{b}R^{c}, —N^{b}R^{c}, —N^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —N^{b}C(═O)OR^{c}, —NR^{b}C(═O)R^{c}, —NR^{b}C(═S)R^{c}, —NR^{b}C(═S)N^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{c}R^{c}, —R^{b}NR^{c}C(═O)R^{b}, —R^{b}OR^{c}, —R^{b}C(═O)OR^{c}, —R^{c}C(═O)NR^{c}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}C(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2.

7. The compound of anyone of claims 1-6, wherein Cy² is selected form 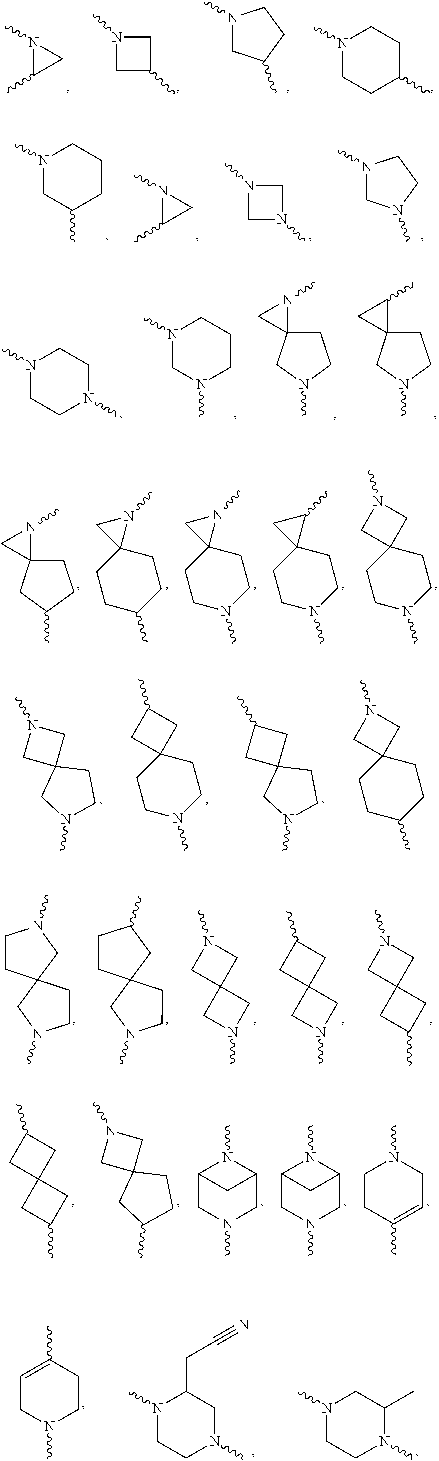 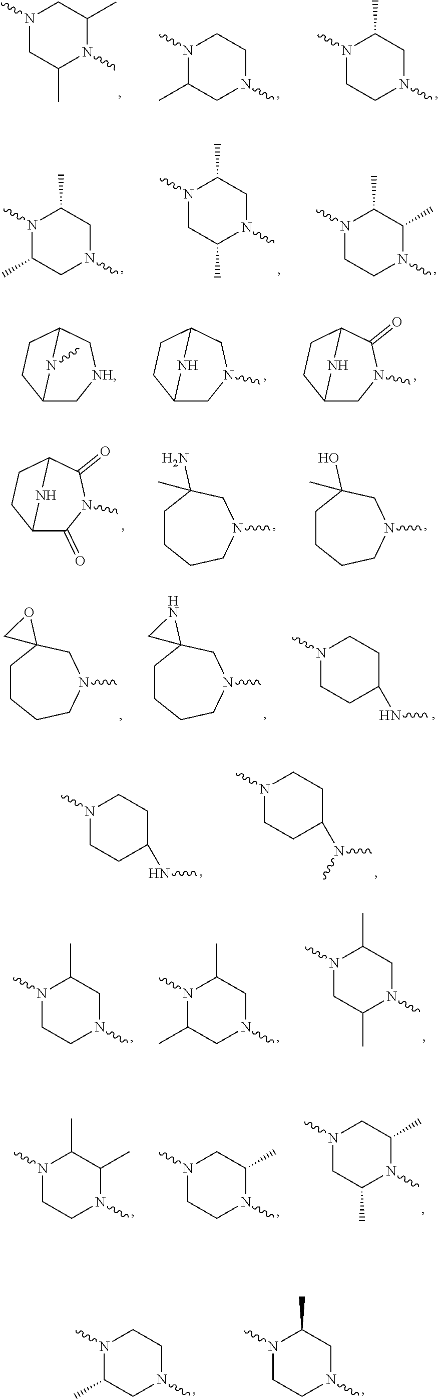 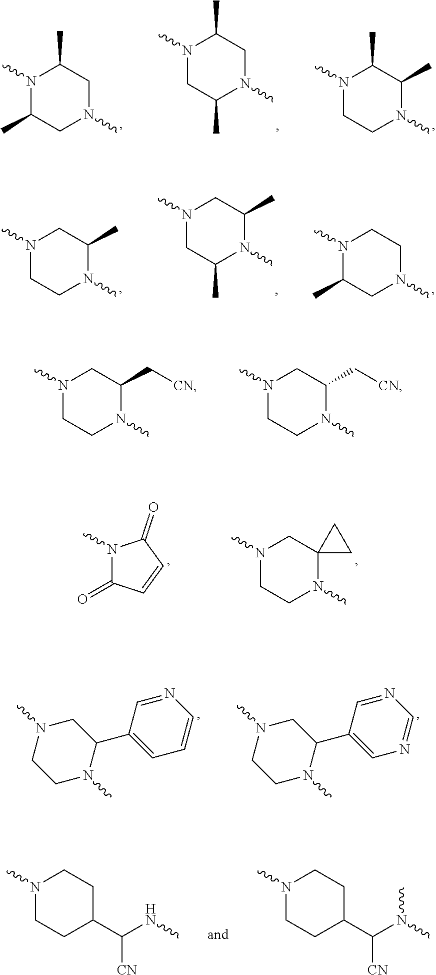
wherein the Cy² is substituted with one or more G

8. The compound of anyone of claims 1-7, wherein A₂—Cy²-G is selected from

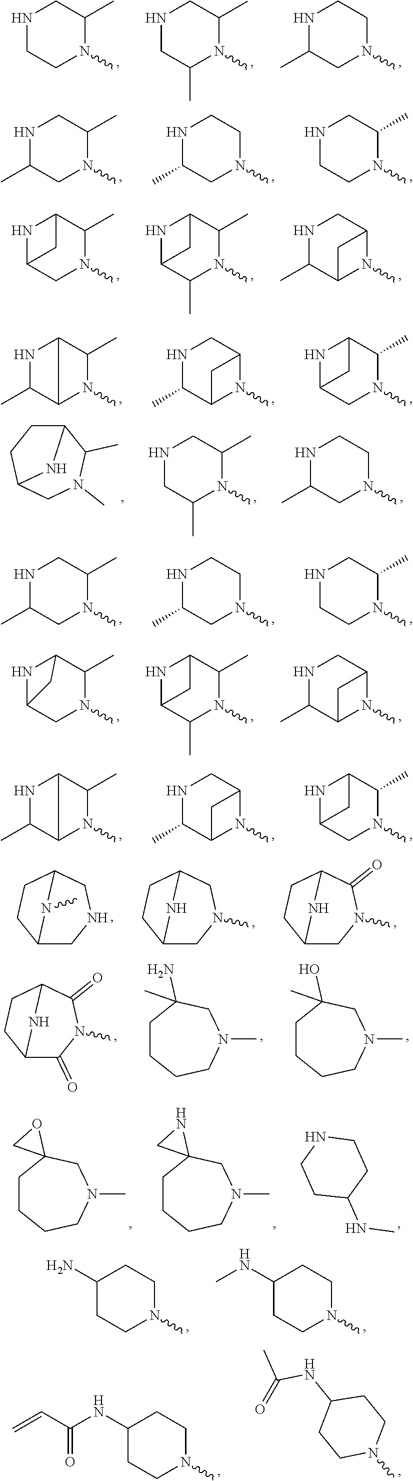

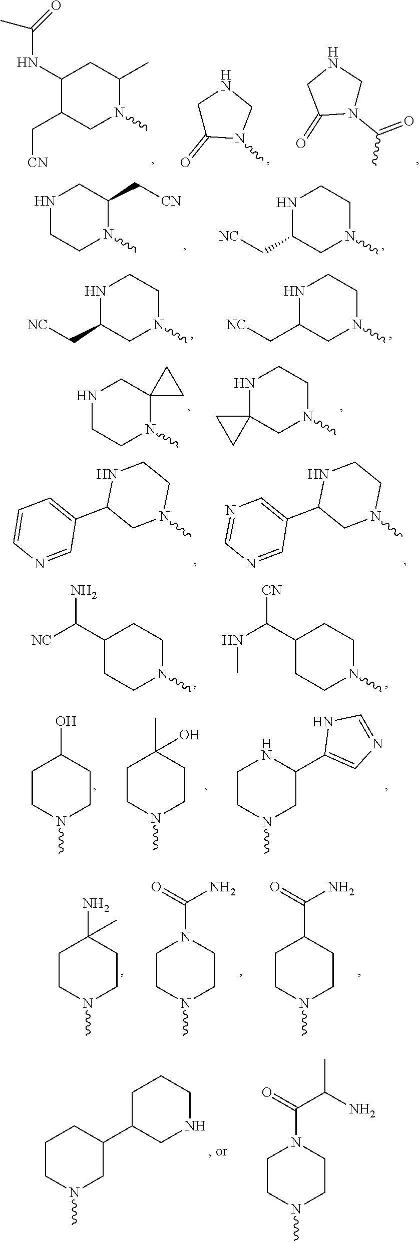

9. A compound of formula (A-IIIA), (A-IIIB) or (A-IIIC) 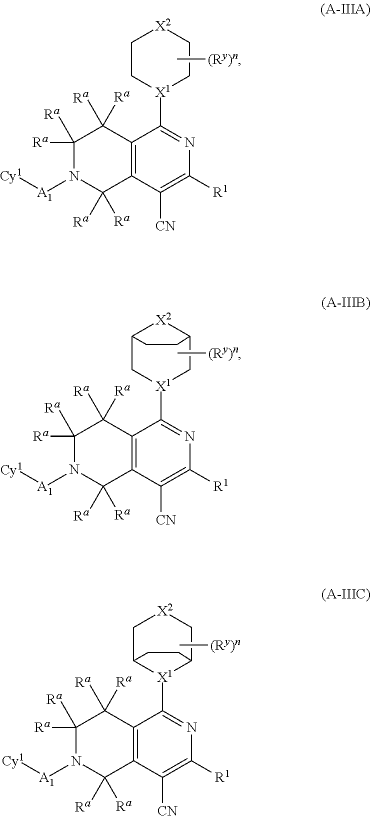
or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
X¹ is C or N;
X² is selected from —N-G or CR^{y}R^{y}-G, O or S;
G is a group capable of forming an interaction with Aspartic acid at 12 position of KRAS protein;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —N^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which maybe same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{y} is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —WR, —NR^{b}—OR^{c}, —NR^{b}C(═O)NR^{b}R^{c}, —NR^{b}S(═O)R^{c}, —NRWS(═O)₂R^{c}, —NR^{b}—OR^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
n is 0,1,2,3,4,5,6,7 or 8; and
each occurrence of p is independently 0, 1 or 2.

10. The compound of anyone of claims 1-9, wherein A₁ is absent or is —CR^{b}R^{c}—; wherein each of R^{b} and R^{c} is independently selected from hydrogen or substituted or unsubstituted alkyl.

11. The compound of anyone of claims 1-10, wherein A₁ is absent or is —CR^{b}R^{c}—; wherein R^{b} is independently methyl or ethyl and R^{c} is hydrogen.

12. The compound of anyone of claims 1-11, wherein A₁ is absent or is —CR^{b}R^{c}—; wherein R^{b} is hydrogen and R^{c} is hydrogen.

13. The compound of anyone of claims 1-12, wherein A₁ is absent or —CH₂ or —(C═O)

14. The compound of anyone of claims 1-13, wherein Cy¹ is selected form a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl.

15. The compound of anyone of claims 1-14, wherein Cy¹ is selected form a cyclic group selected from substituted or unsubstituted aryl and substituted or unsubstituted heteroaryl.

16. The compound of anyone of claims 1-15, wherein Cy¹ is selected from

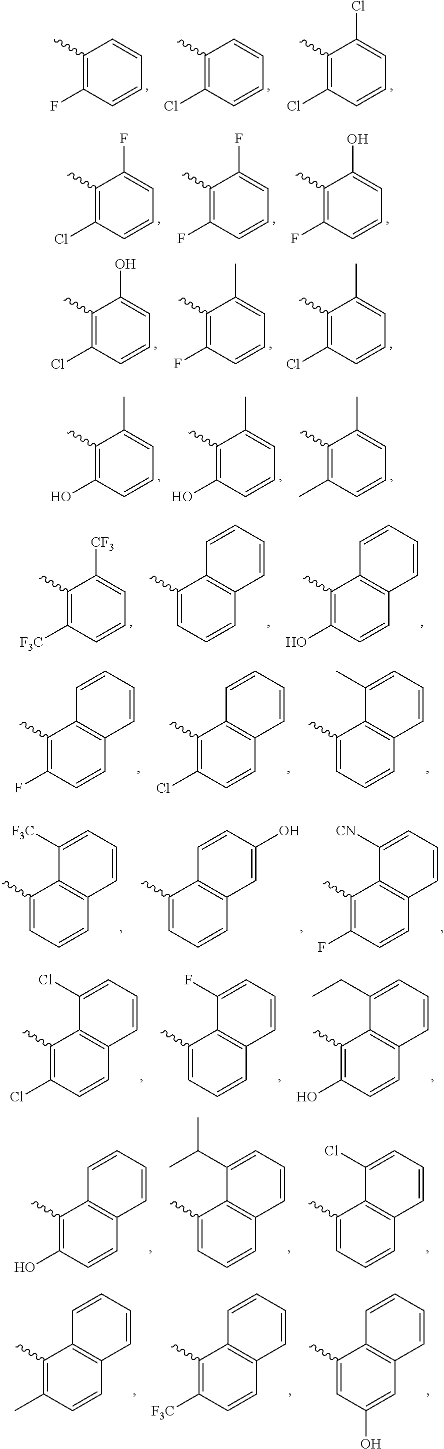

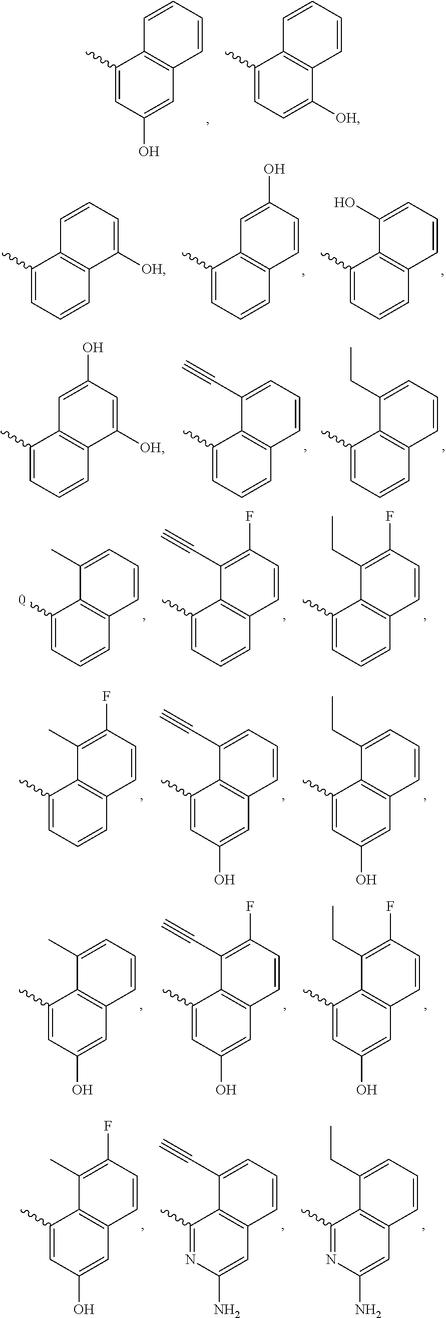

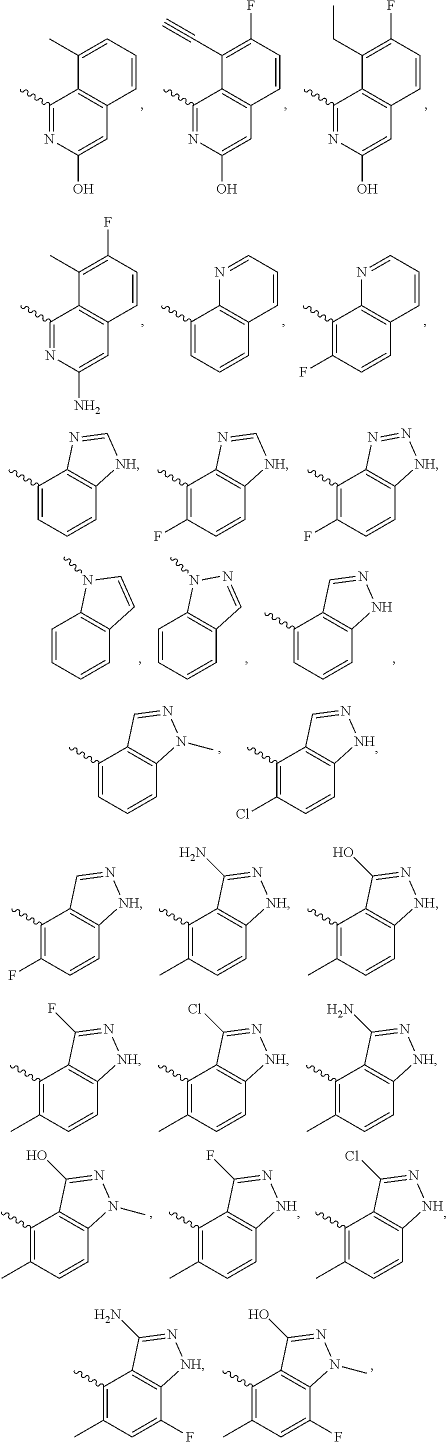

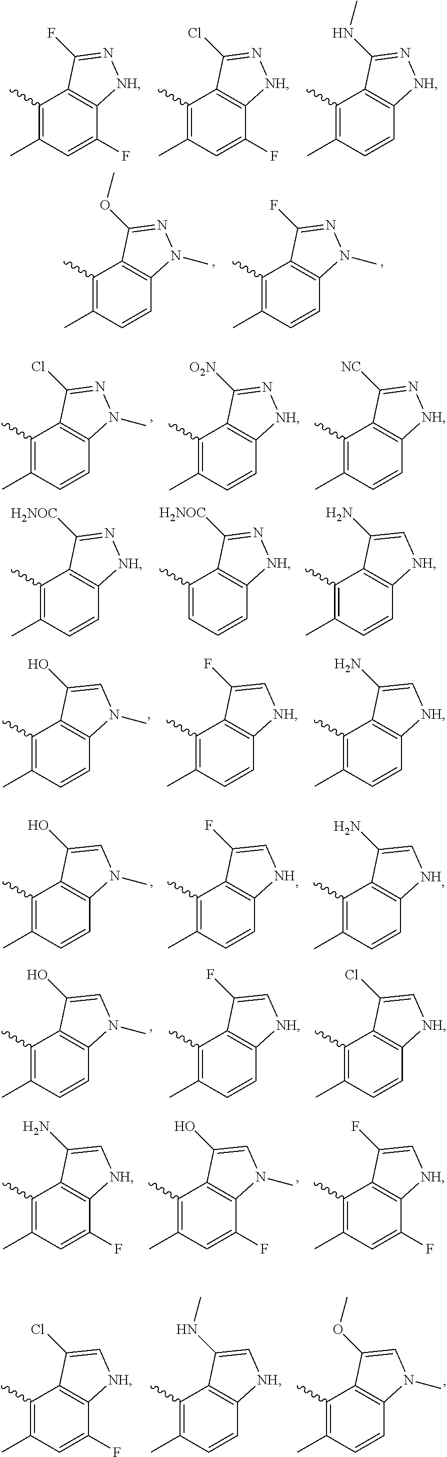

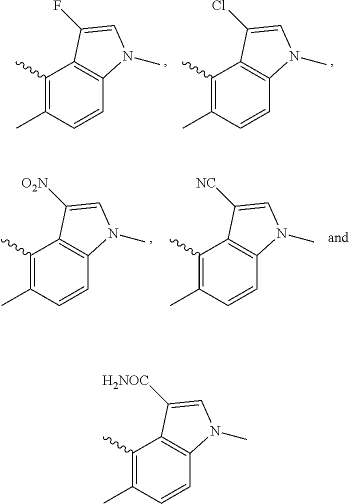

17. The compound of anyone of claims 1-16, wherein G is Hydrogen, —NH₂, —NHR^{a}, —NHOH, —COOH, —OH or —CHR^{a}—OH, or —CH₂—OH or an amino acid.

18. The compound of anyone of claims 1-17, wherein G is Hydrogen, D-Valine or L-threonine

19. The compound of anyone of claims 1-18, wherein R¹ is selected from hydrogen, halogen, OR^{b}, S—R^{b}, —S(═O)pR^{b}—, —C(═O)—R^{b}, —NR^{b}R^{c}, —CO—NR^{b}R^{c}— and —NR^{b}—CO—R^{c}.

20. The compound of anyone of claims 1-19, wherein R¹ is —O—R^{b}.

21. The compound of anyone of claims 1-20, wherein R¹ is —NR^{b}R^{c}.

22. The compound of anyone of claims 1-21, wherein R¹ is independently selected from, substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl.

23. The compound of anyone of claims 1-22, wherein R¹ is independently selected from substituted or unsubstituted alkyl, substituted or unsubstituted heterocyclyl, substituted or unsubstituted heterocyclylalkyl and substituted or unsubstituted heteroaryl.

24. The compound of anyone of claims 1-23, wherein R¹ is independently selected from

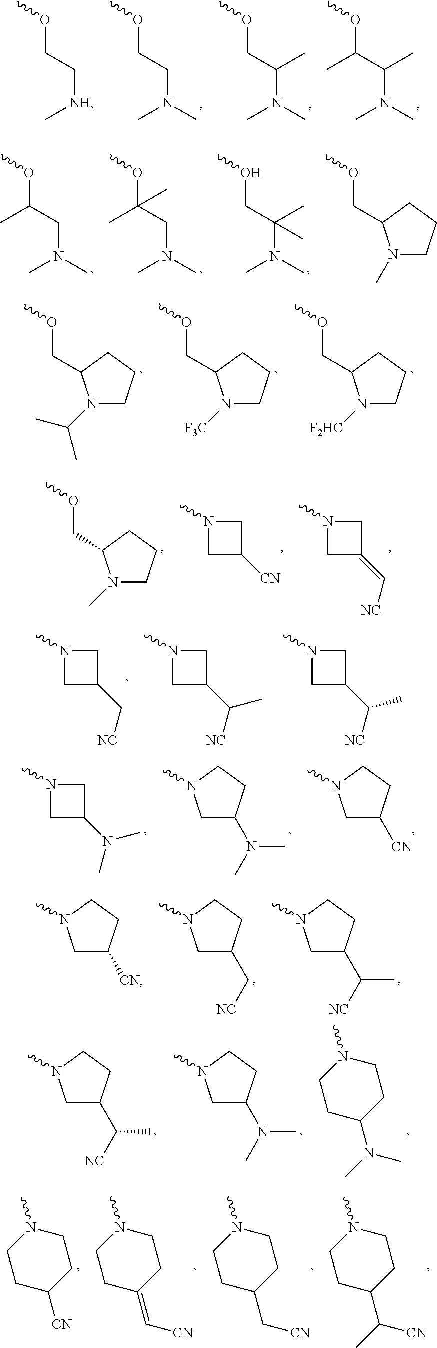

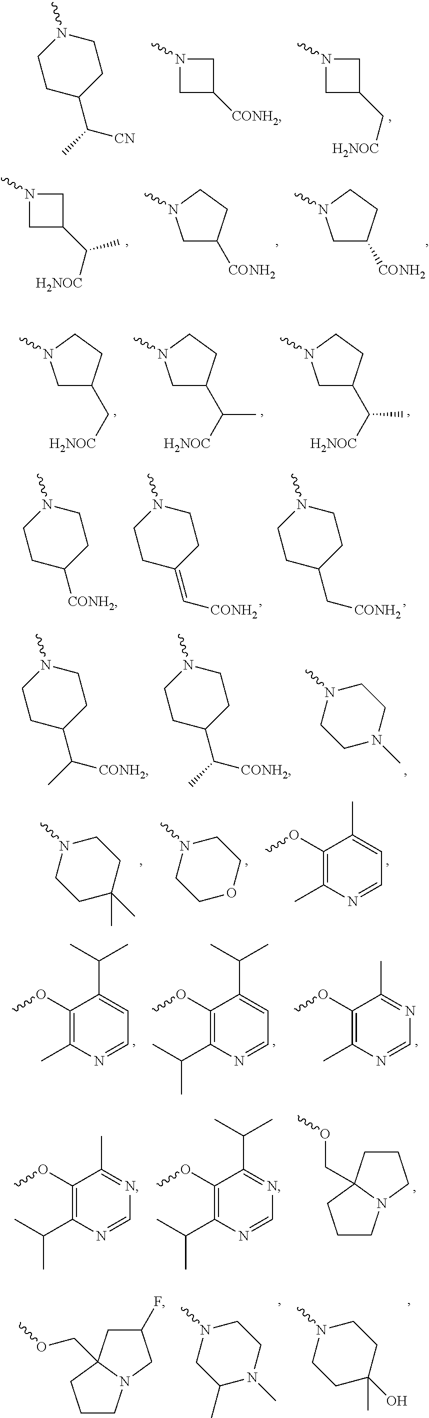

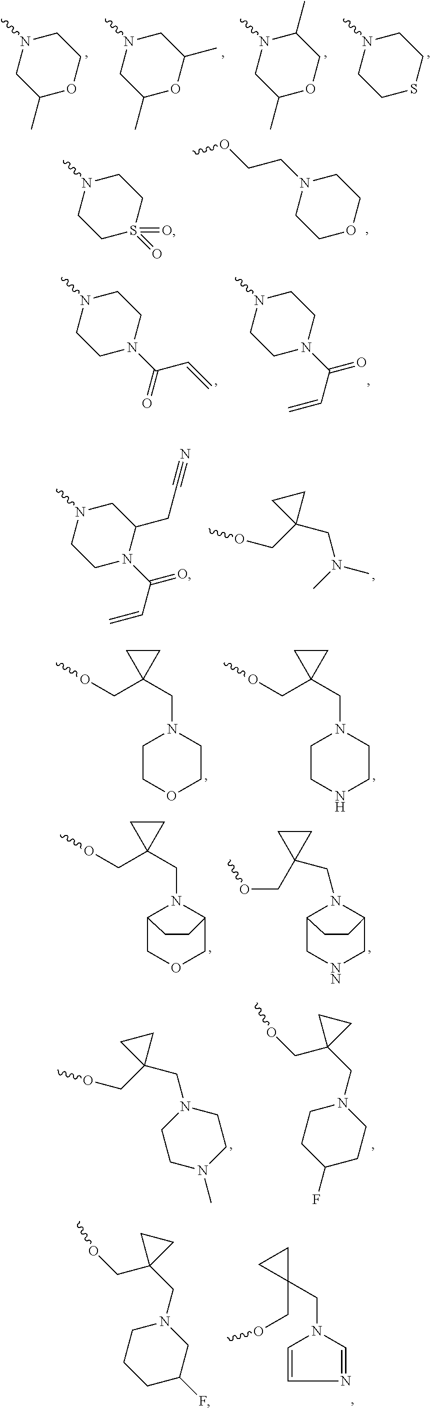

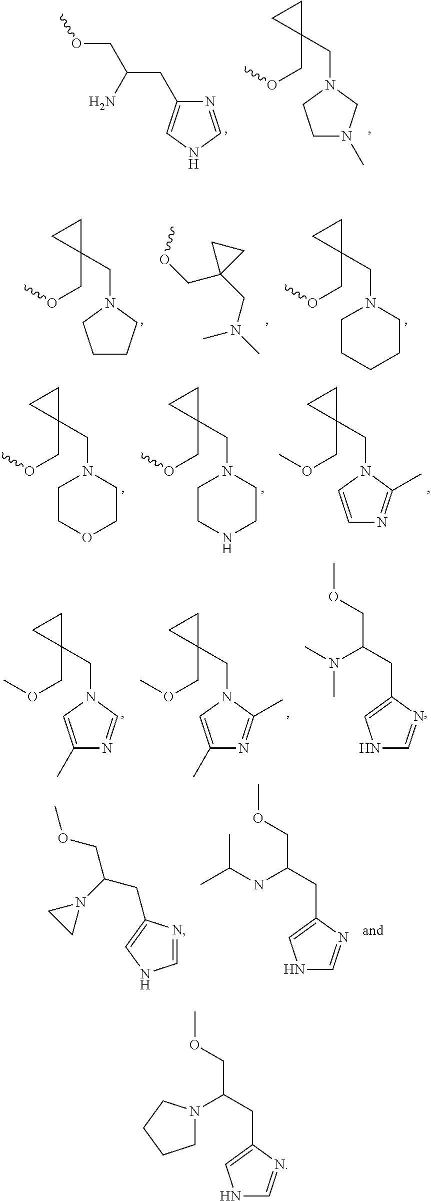

25. The compound of anyone of claims 1-24, wherein R^{a} is Hydrogen or substituted or unsubstituted alkyl.

26. The compound of anyone of claims 1-25, wherein two R^{a} attached to same carbon atom form a C═O (Oxo) group.

27. The compound of anyone of claims 1-26, A₁ is absent or substituted or unsubstituted alkyl;
Cy¹ is selected from substituted or unsubstituted aryl or substituted or unsubstituted heteroaryl;
R¹ is selected from hydrogen, halogen, substituted or unsubstituted alkyl, —NR^{b}R^{c} or —OR^{b};
each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
X¹ is C or N;
X² is selected from —N-G or CH₂-G, O or S;
G is a group capable of forming an interaction with Aspartic acid at 12 position of KRAS protein;
R^{y} is selected from hydrogen, halogen, substituted or unsubstituted alkyl; and
n is 0, 1, 2, 3, 4, 5, 6, 7 or 8.

28. The compound of anyone of claims 1-27, wherein A₁ is methyl.

29. The compound of anyone of claims 1-28, wherein A₁ is absent.

30. The compound of anyone of claims 1-29, wherein Cy¹ is selected from substituted or unsubstituted aryl.

31. The compound of anyone of claims 1-30, wherein Cy¹ is selected from phenyl or naphthalene, optionally substituted with halogen, hydroxy or substituted or unsubstituted alkyl.

32. The compound of anyone of claims 1-31, wherein Cy¹ is selected from quinoline or quinazoline, optionally substituted with halogen, hydroxy or substituted or unsubstituted alkyl.

33. The compound of anyone of claims 1-32, wherein R¹ is selected from hydrogen, halogen, hydroxy,

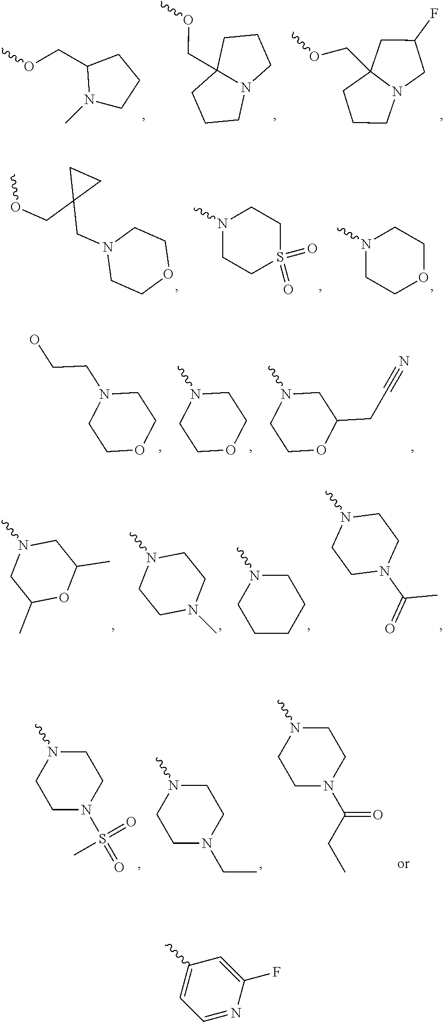

34. The compound of anyone of claims 1-33, wherein X¹ is N.

35. The compound of anyone of claims 1-34, wherein X² is selected from —N-G or CR^{y}R^{y}—G or O, wherein G is selected from Hydrogen, —NH₂, —NHR^{a}, —NHOH, —COOH, —OH or —CHIR^{a}—OH, or —CH₂—OH or an amino acid and R^{y} is Hydrogen.

36. The compound of anyone of claims 1-35, wherein
A₁ is absent or methyl;
Cy¹ is selected from substituted or unsubstituted aryl;
R¹ is selected from hydrogen, halogen, hydroxy, 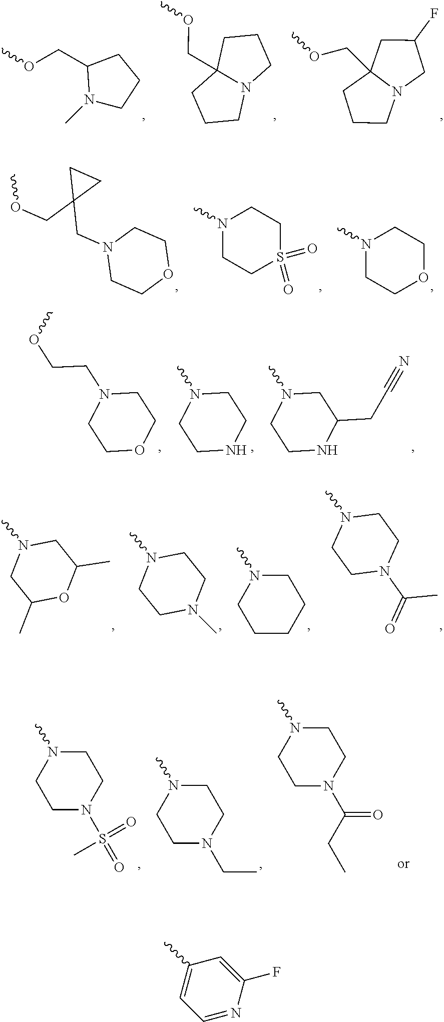
X¹ is N; and
X² is selected from —N-G or CR^{y}R^{y}-G or O, wherein G is selected from Hydrogen, —NH₂, —NHR^{a}, —NHOH, —COOH, —OH or —CHR^{a}—OH, or —CH₂—OH or an amino acid.

37. The compound of anyone of claims 1-36, wherein A₂—Cy²-G is selected from

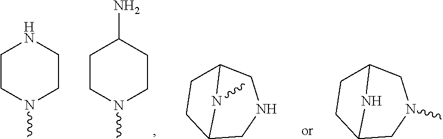

38. A compound selected from 1-(3,8-diazabicyclo[3.2.1]octan-3-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile hydrochloride;
1 tert-butyl 4-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate;
6-(8-chloronaphthalen-1-yl)-1-(3-(cyanomethyl)piperazin-1-yl)-3-(((S)-1-methylpyrrolidin-2-yl)methoxy)-5,6,7,8-tetrahydro-2,6-naphthyridine-4-carbonitrile hydrochloride;
1 tert-butyl (1R,5S)-8-(4-cyano-6-(3-hydroxynaphthalen-1-yl)-3-(((S)-1-methylpyrrolidin-2-yl)methoxy)-5,6,7,8-tetrahydro-2,6-naphthyridin-1-yl)-3, 8-diazabicyclo[3.2.1]octane-3-carboxylate;
1-((1R,5S)-3, 8-diazabicyclo[3.2.1]octan-8-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile hydrochloride;
1-((1R,5S)-3, 8-diazabicyclo[3.2.1]octan-8-yl)-6-(3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
1-((1R,5S)-3, 8-diazabicyclo[3.2.1]octan-8-yl)-6-(8-bromonaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
1-((1R,5S)-3, 8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile compound with 2,2,2-trifluoroacetaldehyde (1:1);
1-((1R,5S,8r)-3-azabicyclo[3.2.1]octan-8-yl)-6-(8-bromo-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile;
1-((1R,5S)-3, 8-diazabicyclo[3.2.1]octan-8-yl)-6-(8-bromonaphthalen-1-yl)-3-((1-methylpyrrolidin-2-yl)methoxy)-5,6,7,8-tetrahydro-2,6-naphthyridine-4-carbonitrile;
1-((1R,5S)-3, 8-diazabicyclo[3.2.1]octan-8-yl)-6-(8-ethynylnaphthalen-1-yl)-3-(((S)-1-methylpyrrolidin-2-yl)methoxy)-5,6,7,8-tetrahydro-2,6-naphthyridine-4-carbonitrile;
1-((1R,5S)-3, 8-diazabicyclo[3.2.1]octan-8-yl)-6-(8-ethynylnaphthalen-1-yl)-3-((tetrahydro-1H-pyrrolizin-7a(5H)-yl)methoxy)-5,6,7,8-tetrahydro-2,6-naphthyridine-4-carbonitrile;
6-benzyl-1-((1R,5S)-3,8-diazabicyclo[3.2.1]octan-8-yl)-3-((1-(morpholinomethyl)cyclopropyl)methoxy)-5,6,7,8-tetrahydro-2,6-naphthyridine-4-carbonitrile;
1-(3,8-diazabicyclo[3.2.1]octan-3-yl)-6-(8-ethynyl-3-hydroxynaphthalen-1-yl)-3-((tetrahydro-1H-pyrrolizin-7a(5H)-yl)methoxy)-5,6,7,8-tetrahydro-2,6-naphthyridine-4-carbonitrile;
1-(3,8-diazabicyclo[3.2.1]octan-3-yl)-6-(8-bromo-3-hydroxynaphthalen-1-yl)-3-(((S)-1-methylpyrrolidin-2-yl)methoxy)-5,6,7,8-tetrahydro-2,6-naphthyridine-4-carbonitrile;
1-(3,8-diazabicyclo[3.2.1]octan-3-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-(3,8-diazabicyclo[3.2.1]octan-3-yl)-6-(8-chloronaphthalen-1-yl)-3-(((2R)-2-fluorotetrahydro-1H-pyrrolizin-7a(5H)-yl)methoxy)-5,6,7,8-tetrahydro-2,6-naphthyridine-4-carbonitrile;
6-benzyl-1-(3,8-diazabicyclo[3.2.1]octan-3-yl)-3-(((S)-1-methylpyrrolidin-2-yl)methoxy)-5,6,7,8-tetrahydro-2,6-naphthyridine-4-carbonitrile;
6-benzyl-1-(3,8-diazabicyclo[3.2.1]octan-3-yl)-3-(((2R)-2-fluorotetrahydro-1H-pyrrolizin-7a(5H)-yl)methoxy)-5,6,7,8-tetrahydro-2,6-naphthyridine-4-carbonitrile;
1 tert-butyl ((R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-methyl-1-oxobutan-2-yl)carbamate;
1-(4-(D-valyl)piperazin-1-yl)-6-benzyl-3-(((S)-1-methylpyrrolidin-2-yl)methoxy)-5,6,7,8-tetrahydro-2,6-naphthyridine-4-carbonitrile;
1 tert-butyl ((2S,3R)-1-(4-(6-benzyl-4-cyano-3-(((S)-1-methylpyrrolidin-2-yl)methoxy)-5,6,7,8-tetrahydro-2,6-naphthyridin-1-yl)piperazin-1-yl)-3-(benzyloxy)-1-oxobutan-2-yl)carbamate;
6-benzyl-1-(4-(O-benzyl-L-threonyl)piperazin-1-yl)-3-(((S)-1-methylpyrrolidin-2-yl)methoxy)-5,6,7,8-tetrahydro-2,6-naphthyridine-4-carbonitrile;
6-benzyl-1-(3,8-diazabicyclo[3.2.1]octan-3-yl)-3-((1-(morpholinomethyl)cyclopropyl)methoxy)-5,6,7,8-tetrahydro-2,6-naphthyridine-4-carbonitrile;
1-(3,8-diazabicyclo[3.2.1]octan-3-yl)-3-(((2R)-2-fluorotetrahydro-1H-pyrrolizin-7a(5H)-yl)methoxy)-6-(3-hydroxynaphthalen-1-yl)-5,6,7,8-tetrahydro-2,6-naphthyridine-4-carbonitrile;
3-(1,1-dioxidothiomorpholino)-6-(naphthalen-1-yl)-1-(piperazin-1-yl)-5,6,7,8-tetrahydro-2,6-naphthyridine-4-carbonitrile Hydrochloride;
6-(8-chloronaphthalen-1-yl)-3-morpholino-1-(piperazin-1-yl)-5,6,7,8-tetrahydro-2,6-naphthyridine-4-carbonitrile Hydrochloride;
3-(2-morpholinoethoxy)-6-(naphthalen-1-yl)-1-(piperazin-1-yl)-5,6,7,8-tetrahydro-2,6-naphthyridine-4-carbonitrile Hydrochloride;
6-(8-chloronaphthalen-1-yl)-3-(2-morpholinoethoxy)-1-(piperazin-1-yl)-5,6,7,8-tetrahydro-2,6-naphthyridine-4-carbonitrile Hydrochloride;
1-(2,6-dimethylmorpholino)-6-(naphthalen-1-yl)-3-(piperazin-1-yl)-5,6,7,8-tetrahydro-2,6-naphthyridine-4-carbonitrile Hydrochloride;
6-(8-chloronaphthalen-1-yl)-1-(2,6-dimethylmorpholino)-3-(piperazin-1-yl)-5,6,7,8-tetrahydro-2,6-naphthyridine-4-carbonitrile Hydrochloride;
3-(3-(cyanomethyl)piperazin-1-yl)-1-(2,6-dimethylmorpholino)-6-(naphthalen-1-yl)-5,6,7,8-tetrahydro-2,6-naphthyridine-4-carbonitrile Hydrochloride;
(S)-6-(8-chloronaphthalen-1-yl)-3-((1-methylpyrrolidin-2-yl)methoxy)-1-(piperazin-1-yl)-5,6,7,8-tetrahydro-2,6-naphthyridine-4-carbonitrile Hydrochloride;
(S)-6-(3-hydroxynaphthalen-1-yl)-3-((1-methylpyrrolidin-2-yl)methoxy)-1-(piperazin-1-yl)-5,6,7,8-tetrahydro-2,6-naphthyridine-4-carbonitrile Hydrochloride;
(S)-3-((1-methylpyrrolidin-2-yl)methoxy)-1-(piperazin-1-yl)-6-(quinazolin-4-yl)-5,6,7,8-tetrahydro-2,6-naphthyridine-4-carbonitrile Hydrochloride;
6-(8-chloronaphthalen-1-yl)-1-(3-methylpiperazin-1-yl)-3-(((S)-1-methylpyrrolidin-2-yl)methoxy)-5,6,7,8-tetrahydro-2,6-naphthyridine-4-carbonitrile Hydrochloride;
1-(3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
1-((S)-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
1-((R)-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile Hydrochloride;
1-(3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
1-((S)-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
1-((R)-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile Hydrochloride;
1-(3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile Hydrochloride;
(S)-6-(isoquinolin-4-yl)-3-((1-methylpyrrolidin-2-yl)methoxy)-1-(piperazin-1-yl)-5,6,7,8-tetrahydro-2,6-naphthyridine-4-carbonitrile Hydrochloride;
(S)-3-((1-methylpyrrolidin-2-yl)methoxy)-1-(piperazin-1-yl)-6-(quinolin-8-yl)-5,6,7,8-tetrahydro-2,6-naphthyridine-4-carbonitrile Hydrochloride;
6-(8-chloronaphthalen-1-yl)-1-(3-(cyanomethyl)piperazin-1-yl)-3-(((S)-1-methylpyrrolidin-2-yl)methoxy)-5,6,7,8-tetrahydro-2,6-naphthyridine-4-carbonitrile Hydrochloride;
6-(8-chloronaphthalen-1-yl)-1-((S)-3-(cyanomethyl)piperazin-1-yl)-3-(((S)-1-methylpyrrolidin-2-yl)methoxy)-5,6,7,8-tetrahydro-2,6-naphthyridine-4-carbonitrile Hydrochloride;
6-(8-chloronaphthalen-1-yl)-1-((S)-3-(cyanomethyl)piperazin-1-yl)-3-(((S)-1-methylpyrrolidin-2-yl)methoxy)-5,6,7,8-tetrahydro-2,6-naphthyridine-4-carbonitrile Hydrochloride;
6-benzyl-3-chloro-1-(piperazin-1-yl)-5,6,7,8-tetrahydro-2,6-naphthyridine-4-carbonitrile Hydrochloride;
1-(4-aminopiperidin-1-yl)-6-benzyl-3-chloro-5,6,7,8-tetrahydro-2,6-naphthyridine-4-carbonitrile Hydrochloride;
1-(4-aminopiperidin-1-yl)-6-benzyl-5,6,7,8-tetrahydro-2,6-naphthyridine-4-carbonitrile Hydrochloride;
6-benzyl-1,3-di(piperazin-1-yl)-5,6,7,8-tetrahydro-2,6-naphthyridine-4-carbonitrile Hydrochloride;
6-benzyl-3-hydroxy-1-(piperazin-1-yl)-5,6,7,8-tetrahydro-2,6-naphthyridine-4-carbonitrile Hydrochloride;
6-benzyl-3-morpholino-1-(piperazin-1-yl)-5,6,7,8-tetrahydro-2,6-naphthyridine-4-carbonitrile Hydrochloride;
6-benzyl-3-(4-methylpiperazin-1-yl)-1-(piperazin-1-yl)-5,6,7,8-tetrahydro-2,6-naphthyridine-4-carbonitrile Hydrochloride;
6-benzyl-1-(3-(cyanomethyl)piperazin-1-yl)-3-(4-methylpiperazin-1-yl)-5,6,7,8-tetrahydro-2,6-naphthyridine-4-carbonitrile Hydrochloride;
6-(1-naphthoyl)-1-(3-(cyanomethyl)piperazin-1-yl)-3-(4-methylpiperazin-1-yl)-5,6,7,8-tetrahydro-2,6-naphthyridine-4-carbonitrile Hydrochloride;
(S)-6-benzyl-1-(2-methylpiperazin-1-yl)-3-(4-methylpiperazin-1-yl)-5,6,7,8-tetrahydro-2,6-naphthyridine-4-carbonitrile Hydrochloride;
(R)-6-benzyl-1-(2-methylpiperazin-1-yl)-3-(4-methylpiperazin-1-yl)-5,6,7,8-tetrahydro-2,6-naphthyridine-4-carbonitrile Hydrochloride;
3-(4-methylpiperazin-1-yl)-6-(naphthalen-1-yl)-1-(piperazin-1-yl)-5,6,7,8-tetrahydro-2,6-naphthyridine-4-carbonitrile Hydrochloride;
6-benzyl-1-(piperazin-1-yl)-3-(piperidin-1-yl)-5,6,7,8-tetrahydro-2,6-naphthyridine-4-carbonitrile Hydrochloride;
6-benzyl-3-(2-fluoropyridin-4-yl)-1-(piperazin-1-yl)-5,6,7,8-tetrahydro-2,6-naphthyridine-4-carbonitrile;
6-(naphthalen-1-yl)-1-(piperazin-1-yl)-3-(piperidin-1-yl)-5,6,7,8-tetrahydro-2,6-naphthyridine-4-carbonitrile Hydrochloride;
6-benzyl-3-((1-methylpyrrolidin-2-yl)methoxy)-1-(piperazin-1-yl)-5,6,7,8-tetrahydro-2,6-naphthyridine-4-carbonitrile Hydrochloride;
6-(naphthalen-1-yl)-1,3-di(piperazin-1-yl)-5,6,7,8-tetrahydro-2,6-naphthyridine-4-carbonitrile Hydrochloride;
6-(benzo[b]thiophen-4-yl)-1,3-di(piperazin-1-yl)-5,6,7,8-tetrahydro-2,6-naphthyridine-4-carbonitrile dihydrochloride;
3-morpholino-6-(naphthalen-1-yl)-1-(piperazin-1-yl)-5,6,7,8-tetrahydro-2,6-naphthyridine-4-carbonitrile Hydrochloride;
3-(4-acetylpiperazin-1-yl)-6-(naphthalen-1-yl)-1-(piperazin-1-yl)-5,6,7,8-tetrahydro-2,6-naphthyridine-4-carbonitrile Hydrochloride;
3-(2,6-dimethylmorpholino)-6-(naphthalen-1-yl)-1-(piperazin-1-yl)-5,6,7,8-tetrahydro-2,6-naphthyridine-4-carbonitrile Hydrochloride;
3-(4-(methylsulfonyl)piperazin-1-yl)-6-(naphthalen-1-yl)-1-(piperazin-1-yl)-5,6,7,8-tetrahydro-2,6-naphthyridine-4-carbonitrile Hydrochloride;
3-(4-ethylpiperazin-1-yl)-6-(naphthalen-1-yl)-1-(piperazin-1-yl)-5,6,7,8-tetrahydro-2,6-naphthyridine-4-carbonitrile Hydrochloride;
(S)-3-((1-methylpyrrolidin-2-yl)methoxy)-6-(naphthalen-1-yl)-1-(piperazin-1-yl)-5,6,7,8-tetrahydro-2,6-naphthyridine-4-carbonitrile Hydrochloride;
6-(naphthalen-1-yl)-1-(piperazin-1-yl)-5,6,7,8-tetrahydro-2,6-naphthyridine-4-carbonitrile Hydrochloride;
6-(naphthalen-1-yl)-1-(piperazin-1-yl)-3-(4-propionylpiperazin-1-yl)-5,6,7,8-tetrahydro-2,6-naphthyridine-4-carbonitrile Hydrochloride; and
6-benzyl-1-((S)-2-methylpiperazin-1-yl)-3-((1-methylpyrrolidin-2-yl)methoxy)-5,6,7,8-tetrahydro-2,6-naphthyridine-4-carbonitrile and pharmaceutically acceptable salts thereof.

39. A Pharmaceutical composition comprising a compound of claim 1-38, or a pharmaceutically acceptable salt thereof, and a pharmaceutically acceptable carrier.

40. A method for treating cancer in a subject in need thereof, the method comprising administering to a subject in need thereof a therapeutically effective amount of a compound of claim 1-38, or a pharmaceutically acceptable salt thereof, or a composition comprising a compound of claim 1-38, or a pharmaceutically acceptable salt thereof.

41. The method of claim 40, further comprising the step of administering simultaneously or sequentially to a subject in need thereof at least one other anti-cancer agent, anti-inflammatory agent, immunosuppressive agent, steroid, non-steroidal anti-inflammatory agent, antihistamine, analgesic, or a mixture thereof.

42. A method of treating a KRAS mediated disorder in a subject in need thereof, in particular KRAS G12D mediated disorder comprising administering to the subject in need thereof a therapeutically effective amount of a compound of claim 1-38, or a pharmaceutically acceptable salt thereof, or a pharmaceutical composition comprising a compound of claim 1-38, or a pharmaceutically acceptable salt thereof.

43. Use of a compound of claim 1-38, for treating cancer or a RAS-mediated disorder, in particular KRAS G12D mediated cancer or disorder in a subject.

44. Use of a compound of claim 1-38 for the manufacture of a medicament for treating cancer or a RAS mediated disorder, in particular KRAS G12D mediated cancer or disorder in a subject.

45. A method of treating a variety of cancers, such as solid cancer and, more specifically, solid cancers with KRASG12 mutation wherein the method comprises of administering to a subject a Compounds of claim 1-38, or the pharmaceutically acceptable salts and/or compositions thereof.

46. The method of claim 40-45, wherein cancer is selected from Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

47. The method of claim 40-46, wherein the cancer is lung cancer, pancreatic cancer, or colorectal cancer.

48. The method of claim 47, wherein the cancer is lung cancer.

49. The method of claim 47, wherein the cancer is non-small cell lung cancer.

50. The method of claim 47, wherein the cancer is pancreatic cancer.

51. The method of claim 47, wherein the cancer is colorectal cancer.
